(19) United States
(12) Patent Application Publication
(10) Pub. No.: US 2010/0292173 A1
(43) Pub. Date: Nov. 18, 2010

(54) Wound Healing Compounds And Methods

(75) Inventor: Donna Livant, Ann Arbor, MI (US)

Correspondence Address: Medlen and Carroll, LLP, Suite 350, 101 Howard Street, San Francisco, CA, 94105, US

(73) Assignee: The Regents Of The University Of Michigan

(21) Appl. No.: 12/776,717

(22) Filed: May 10, 2010

Related U.S. Application Data
(63) Continuation of application No. 10/964,093, filed on Oct. 13, 2004.

(51) Int. Cl. A61K 38/08; C07K 7/06; A61P 17/02

(52) U.S. Cl. 514/21.8; 530/330

(57) ABSTRACT

The testing of tumor cells, including human tumors capable of metastases, in assays employing fibronectin-depleted substrates is described. Ex vivo induction of cells, including biopsied human cells, is performed with invasion-inducing agents. Additionally, anti-cancer chemotherapeutics are described. Specifically, chemotherapeutic agents which have anti-metastatic and anti-growth properties are described including non-peptide compositions of matter.

### FIELD OF THE INVENTION

The present invention relates to the treatment of cancer, to the testing of cancer cells for their ability to invade tissues and cause metastases, and to the identification and use of drugs to inhibit tumor invasion and growth.

### BACKGROUND

The term “chemotherapy” simply means the treatment of disease with chemical substances. The father of chemotherapy, Paul Ehrlich, imagined the perfect chemotherapeutic as a “magic bullet”; such a compound would kill invading organisms without harming the host. This target specificity is sought in all types of chemotherapeutics, including anticancer agents.

However, specificity has been the major problem with anticancer agents. In the case of anticancer agents, the drug needs to distinguish between host cells that are cancerous and host cells that are not cancerous. The vast bulk of anticancer drugs are indiscriminate at this level. Typically anticancer agents have negative hematological effects (e.g., cessation of mitosis and disintegration of formed elements in marrow and lymphoid tissues), and immunosuppressive action (e.g., depressed cell counts), as well as a severe impact on epithelial tissues (e.g., intestinal mucosa), reproductive tissues (e.g., impairment of spermatogenesis), and the nervous system. P. Calabresi and B. A. Chabner, In: Goodman and Gilman, The Pharmacological Basis of Therapeutics (Pergamon Press, 8th Edition) (pp. 1209-1216).

Success with chemotherapeutics as anticancer agents has also been hampered by the phenomenon of multiple drug resistance, resistance to a wide range of structurally unrelated cytotoxic anticancer compounds. Gerlach et al., Cancer Surveys, 5:25-46 (1986). The underlying cause of progressive drug resistance may be due to a small population of drug-resistant cells within the tumor (e.g., mutant cells) at the time of diagnosis. Goldie et al., Cancer Research, 44:3643-3653 (1984). Treating such a tumor with a single drug first results in a remission, where the tumor shrinks in size as a result of the killing of the predominant drug-sensitive cells. With the drug-sensitive cells gone, the remaining drug-resistant cells continue to multiply and eventually dominate the cell population of the tumor.

Finally, the treatment of cancer has been hampered by the fact that there is considerable heterogeneity even within one type of cancer. Some cancers, for example, have the ability to invade tissues and display an aggressive course of growth characterized by metastases. These tumors generally are associated with a poor outcome for the patient. And yet, without a means of identifying such tumors and distinguishing such tumors from non-invasive cancer, the physician is at a loss to change and/or optimize therapy.

What is needed is a specific anticancer approach that is reliable for a wide variety of tumor types, and particularly suitable for invasive tumors. Importantly, the treatment must be effective with minimal host toxicity.

### SUMMARY OF THE INVENTION

The present invention relates to the treatment of cancer, to the testing of cancer cells for their ability to invade tissues and cause metastases, and to the identification and use of drugs to inhibit tumor invasion and growth.

In one embodiment, the present invention contemplates a composition comprising a dendrimer and at least one peptide comprising an amino acid sequence PHSCN attached to said dendrimer, wherein the dendrimer comprises branches. In one embodiment, the dendrimer comprises polylysine. In one embodiment, the composition further comprises a chemotherapeutic agent attached to the dendrimer. In one embodiment, the chemotherapeutic agent comprises methotrexate. In another embodiment, the chemotherapeutic agent comprises boron. In another embodiment, the chemotherapeutic agent comprises an antibody. In another embodiment, the chemotherapeutic agent comprises a receptor. In another embodiment, the chemotherapeutic agent comprises gemcitabine. In another embodiment, the chemotherapeutic agent comprises 5-fluoruracil. In another embodiment, the chemotherapeutic agent comprises a CDK inhibitor. In another embodiment, the chemotherapeutic agent comprises a matrix metalloproteinase inhibitor. In another embodiment, the chemotherapeutic agent comprises cisplatin. In another embodiment, the chemotherapeutic agent comprises doxorubicin. In another embodiment, the chemotherapeutic agent comprises estramustine. In another embodiment, the chemotherapeutic agent comprises etoposide. In another embodiment, the chemotherapeutic agent comprises docetaxel. In another embodiment, the chemotherapeutic agent comprises paclitaxel. In another embodiment, the chemotherapeutic agent comprises tamoxifen. In another embodiment, the chemotherapeutic agent comprises vincristine. In another embodiment, the composition is attached to a tumor cell. In one embodiment, the tumor cell further comprises α5β1 integrin. In another embodiment, the tumor cell is associated with blood vessels.

In one embodiment, the present invention contemplates a method, comprising: a) providing; i) a patient comprising a plurality of tumor cells; and ii) a composition comprising a dendrimer and at least one peptide comprising an amino acid sequence PHSCN attached to said dendrimer wherein said composition is capable of inducing apoptosis or inhibiting collagenase-dependent and/or matrix metalloproteinase-dependent invasion; and b) administering said composition to said patient under conditions such that at least a portion of said tumor cells undergo apoptosis or are preventing from invading. In one embodiment, the tumor cells comprise prostate tumor cells. In one embodiment, the patient further comprises tumor-associated blood vessel cells. In another embodiment, the patient further comprises tumor-associated lymphatic vessels. In one embodiment, the blood vessel cells comprise endothelial cells. In another embodiment, the lymphatic vessel cells comprise endothelial cells. In one embodiment, the composition induces apopotosis in said endothelial cells, or prevents their invasion of the tumor (angiogenesis). In one embodiment, the dendrimer further comprises a chemotherapeutic agent, wherein the agent is attached to the dendrimer. In one embodiment, the chemotherapeutic agent is selected from the group consisting of methotrexate, boron, cisplatin, doxorubicin, estramustine, etoposide, gemcitabine, 5-fluorouracil, paclitaxel, tamoxifen, vincristine, an antibody, and a receptor. In one embodiment, the apoptosis is caused by focal adhesion kinase inhibition. In one embodiment, the apoptosis is caused by protein kinase B inhibition. In another embodiment, invasion inhibition is caused by the inhibition of matrix metalloproteinase activity and/or expression by the tumor cells or the host endothelial cells of blood or lymphatic vessels.

In one embodiment, the present invention contemplates a method, comprising: a) providing; i) a patient comprising a plurality of metastatic tumor cells; and ii) a composition comprising a dendrimer and at least one peptide comprising an amino acid sequence PHSCN attached to said dendrimer wherein said composition is capable of inhibiting metastatic activity; and b) administering said composition to said patient under conditions such that the metastatic activity by said tumor cells is inhibited. In one embodiment, the tumor cells comprise prostate tumor cells. In one embodiment, the tumor cells comprise pancreatic tumor cells. In one embodiment, the patient further comprises tumor-associated blood vessel cells. In one embodiment, the blood vessel cells comprise endothelial cells. In one embodiment, the composition inhibits tumor cell invasion of said endothelial cells. In one embodiment, the dendrimer further comprises a chemotherapeutic agent, wherein the agent is attached to the dendrimer. In one embodiment, the chemotherapeutic agent is selected from the group consisting of methotrexate, boron, an antibody, and a receptor.

The present invention also provides: A) an in vitro model for testing cancer cells and evaluating invasive potential; B) a screening assay for identifying drugs that inhibit tumor invasion; and C) chemotherapeutics for treating cancer.

A variety of assay formats are contemplated for testing the invasive potential of cancer cells. In one embodiment, a portion of a patient's tumor is obtained (e.g., by biopsy) and placed in tissue culture on a fibronectin-free substrate. Thereafter, the response of the tumor cells to fibronectin or a fibronectin-derived peptide is assessed. Where fibronectin induces invasion of the membrane, the tumor can be considered to have metastatic potential. Where there is no significant invasion of the membrane, the tumor can be considered (at that time) to be non-metastatic.

In one embodiment, the present invention contemplates a method of evaluating human cancer comprising: a) providing: i) a human cancer patient, ii) a fibronectin-free substrate, and iii) one or more invasion-inducing agents; b) obtaining cancer cells from said patient; c) contacting said cells ex vivo with said fibronectin-free substrate and one or more invasion-inducing agents; and d) detecting cancer cell invasion of said substrate. Preferably the cancer cells are cultured in serum-free culture media so as to essentially avoid introducing complicating factors. In one embodiment, the invasion-inducing agent is a peptide, said peptide comprising the sequence PHSRN (SEQ ID NO:1). In a preferred embodiment the invasion inducing agent is intact fibronectin.

While not limited to any mechanism, it is believed that cells exposed to invasion-inducing agents in this manner are potentially rendered capable of invading the substrate. Indeed, the present invention contemplates stimulation of invasion by all cells of the body, including, but not limited to: epithelial (keratinocytes, mammary and prostate epithelial), connective tissue (fibroblasts), and muscle (myoblast) cells. Again, while not limited to any mechanism, it is believed that the invasion inducing agent comprising the sequence PHSRN (SEQ ID NO:1) binds to the α5β1 receptor on the cancer cell and thereby induces invasion of the substrate. In this regard, the present invention provides a method of testing human cancer cells comprising: a) providing: i) a human cancer patient, ii) a fibronectin-free substrate, and iii) one or more invasion-inducing agents; b) obtaining α5β1 integrin fibronectin receptor-expressing cancer cells from said patient; c) culturing said cells in serum-free culture media on said substrate in the presence of said invasion-inducing agents; and d) detecting cancer cell invasion of said substrate.

As noted above, the present invention also contemplates a screening assay for identifying drugs that inhibit tumor invasion. The present invention contemplates a screening assay utilizing the binding activity of fibronectin-derived peptides. In one embodiment, an inducible tumor cell line is placed in tissue culture on a fibronectin-free substrate. Thereafter, as an inducible tumor cell line, the tumor will be induced (under ordinary conditions) by fibronectin or the fibronectin-derived peptide to invade the substrate. However, in this drug screening assay, candidate drug inhibitors are added to the tissue culture (this can be done individually or in mixtures). Where the inducible tumor cell is found to be inhibited from invading the substrate, a drug inhibitor is indicated.

It is not intended that the present invention be limited by the nature of the drugs screened in the screening assay of the present invention. A variety of drugs, including peptides, are contemplated.

Finally, the present invention contemplates chemotherapeutics for treating invasive tumors. Specifically, a variety of anti-invasive chemotherapeutic agents are contemplated to antagonize the invasion-promoting activity of the PHSRN (SEQ ID NO:1) peptide. In the preferred embodiment, the anti-invasive agent is a peptide with the amino acid sequence PHSCN (SEQ ID NO:86). In another embodiment, the anti-invasive agent is a peptide which has an amino acid sequence comprising a sequence selected from the group consisting of CHSRN (SEQ ID NO:87), PCSRN (SEQ ID NO:88), PHCRN (SEQ ID NO:89), and PHSRC (SEQ ID NO:90). In another embodiment, the anti-invasive agent is a peptide which has an amino acid sequence comprising PHSXN (SEQ ID NO:91), where X is an amino acid selected from the group consisting of homo-cysteine, the D-isomer of cysteine, histidine, or penicillamine.

The present invention also contemplates an anti-invasive agent comprising the amino acid sequence X₁HSX₂N (SEQ ID NO:92), wherein X₁ is either proline, histidine, or not an amino acid, and X₂ is an amino acid selected from the group consisting of the L-isomer of cysteine, the D-isomer of cysteine, homo-cysteine, histidine, or penicillamine. In another embodiment, the present invention contemplates an anti-invasive agent comprising the amino acid sequence X₁X₂X₃X₄X₅ (SEQ ID NO:93), wherein X₁ is an amino acid selected from the group consisting of proline, glycine, valine, histidine, isoleucine, phenylalanine, tyrosine, and tryptophan, and X₂ is an amino acid selected from the group consisting of histidine, proline, tyrosine, asparagine, glutamine, arginine, lysine, phenylalanine, and tryptophan, and X₃ is an amino acid selected from the group consisting of serine, threonine, alanine, tyrosine, leucine, histidine, asparagine, and glutamine, and X₄ is an amino acid selected from the group consisting of cysteine, homo-cysteine, penicillamine, histidine, tyrosine, asparagine, glutamine, and methionine, and X₅ is an amino acid selected from the group consisting of asparagine, glutamine, serine, threonine, histidine, and tyrosine. In the preferred embodiment the peptide is PHSCN (SEQ ID NO:86), where the cysteine is the L-isomer.

It is further contemplated that the anti-invasive agents named above comprise the named amino acid sequence and additional amino acids added to the amino terminus, the carboxyl terminus, or both the amino and carboxyl termini. In one embodiment, the anti-invasive agent is up to five hundred amino acids in length. It is also contemplated that, in some embodiments, the anti-invasive agents named above comprise a peptide with the amino terminus blocked by standard methods to prevent digestion by exopeptidases, for example by acetylation; and the carboxyl terminus blocked by standard methods to prevent digestion by exopeptidases, for example, by amidation.

In this regard, the present invention provides a method of treating cancer comprising: a) providing: i) a subject having cancer, and ii) a composition of matter comprising a peptide which inhibits the tumor invasion-promoting activity of the PHSRN (SEQ ID NO:1) sequence of plasma fibronectin; and b) administering said composition to said subject. The present invention further contemplates using antagonists before and/or after surgical removal of the primary tumor. In one embodiment, the method comprises administering a PHSRN (SEQ ID NO:1) antagonist as adjunct therapy with additional chemotherapeutics.

While not limited to any mechanism, it is believed that these anti-invasive chemotherapeutic agents antagonize the invasion-promoting activity of the PHSRN (SEQ ID NO:1) sequence (e.g., of fibronectin) by blocking the binding of this sequence to its receptor on tumor cells. Again, while not limited to any mechanism, it is believed that the PHSRN (SEQ ID NO:1) sequence may promote invasion by acting to displace a divalent cation (Mg⁺², Ca⁺², or Mn⁺) in the α5β1 receptor on metastatic tumor cells, and the above named chemotherapeutic anti-invasive agents might act to inhibit this invasion by chelating one or more of these divalent cations.

In another embodiment, the present invention contemplates anti-invasion antagonists to the IKVAV (SEQ ID NO:2) sequence of laminin, including but not limited to, peptides comprising the structure, ICVAV (SEQ ID NO:94), and corresponding peptide mimetics.

### BRIEF DESCRIPTION OF THE FIGURES

FIG. 1 schematically shows the one embodiment of the substrate used according to the present invention for testing tumor cells. The spatial relationship of the ectoderm of the Strongylocentrotus purpuratus embryo to its extracellular matrix and to blastocoelar structures are shown (s, spicules; h, hyalin layer; e, ectoderm; b, subectodermal basement membrane; bl, blastocoel; g, stomach of the primitive gut; c, coelomic pouches). The esophagus and intestine do not appear on the side of the embryo shown.

FIG. 2 is a graph showing the results of the testing of tumor cells on fibronectin-containing substrates and fibronectin-depleted substrates in vitro without the use of the invasion-inducing agents of the present invention.

FIG. 3 is a graph showing the results of the testing of tumor cells on fibronectin-depleted substrates in vitro with and without invasion-inducing agents according one embodiment of the method of the present invention.

FIG. 4 is a graph showing the results of the testing of normal cells on fibronectin-depleted substrates in vitro with and without invasion-inducing agents according one embodiment of the method of the present invention.

FIG. 5A is a graph showing the results of inhibiting serum-induced human breast cancer cell invasion of the SU-ECM substrate with varying concentrations of the PHSCN (SEQ ID NO:86) peptide.

FIG. 5B is a graph showing the results of inhibiting PHSRN (SEQ ID NO:88)-induced invasion by both human breast cancer cells and normal human mammary epithelial cells of the SU-ECM substrate with varying concentrations of the PHSCN (SEQ ID NO:86) peptide.

FIG. 6A is a graph showing the results of inhibiting serum-induced human prostate cancer cell invasion of the SU-ECM substrate with varying concentrations of the PHSCN (SEQ ID NO:86) peptide.

FIG. 6B is a graph showing the results of inhibiting PHSRN-induced invasion by both human prostate cancer cells and normal prostate epithelial cells of the SU-ECM substrate with varying concentrations of the PHSCN (SEQ ID NO:86) peptide.

FIG. 7A is a graph showing the results of testing serum-induced rat prostate cancer cell invasion of the SU-ECM substrate with and without the PHSCN (SEQ ID NO:86) peptide.

FIG. 7B is a graph showing the results of inhibiting PHSRN-induced (SEQ ID NO:1) rat prostate cancer cell invasion of the SU-ECM substrate with varying concentrations of the PHSCN (SEQ ID NO:86) peptide.

FIG. 8 is a graph showing the results of inhibiting serum-induced rat prostate cancer cell invasion of the SU-ECM substrate with varying concentrations of the PHS(homo)CN (SEQ ID NO:85) peptide.

FIG. 9A is a graph showing the results of testing tumor growth in rats injected with prostate cancer cells, with half of the rats receiving treatment with the PHSCN (SEQ ID NO:86) peptide, initiated in conjunction with the initial injection.

FIG. 9B is a graph showing the results of determining the mean number of lung metastases in the two groups of rats described in FIG. 9A.

FIG. 10A is a graph showing the results of testing tumor growth in rats injected with prostate cancer cells, with half of the rats receiving treatment with the PHSCN (SEQ ID NO:86) peptide, initiated 24 hours after the initial cancer cell injection.

FIG. 10B is a graph showing the results of determining the mean number of to lung metastases in the two groups of rats described in FIG. 10A.

FIG. 10C is a graph showing the results of determining the mean mass of intraperitoneal metastatic tissues in the two groups of rats described in FIG. 10A.

FIG. 11 provides exemplary data showing PHSCN saturation binding kinetics for the α5β1 integrin receptor. X Axis: Optical Density (490 nm). Y Axis: PHSCNGGK(biotin) Concentration (nM).

FIG. 12 provides exemplary data showing that a PHSCN peptide may attach to the β1 integrin region on DU 145 cells. Monoclonal antibodies (Mab) 2252 were raised to β1 integrin amino acids 15-54. Monoclonal antibodies (Mab) 2251 were raised to β1 integrin amino acids 657-670. The binding ligand is PHSCNGGK(biotinylated) (SEQ ID NO:105).

FIG. 13 provides exemplary data showing that manganese ion (Mn²⁺) enhances biotinylated PHSCN peptide binding to α5β1 integrin. Squares: Mn²⁺ absent; Circles: Mn²⁺ present.

FIG. 14 provides exemplary Western immunoblot gel electrophoresis data showing that incubation with Ac-PHSCN-NH₂ peptide upregulates Bad and Bax protein expression in adherent DU 145 cells. D1-D7=Days 1-7; Ctrl=Untreated DU 145 cells cultured in the same serum-containing medium, in parallel with the treated cells control cell culture collected on Day 6. Actin=Internal Standard.

FIG. 15A provides exemplary Western immunoblot gel electrophoresis data showing Caspase 9 activation in cell lysates of adherent DU 145 cells incubated with Ac-PHSCN-NH₂ peptide. Upper Gel: Cell Lysate Probed With A Monoclonal Antibody Specific For The Asp³⁰⁰ Epitope Of Caspase 9; D1-D7=Incubation Day. C6=Control Culture tested on Day 6. Middle Gel: Cell Lysate Probed With A Monoclonal Antibody Specific For The Asp³¹⁵ Epitope Of Caspase 9. Lower Gel: Actin Internal Standard, used to demonstrate equal loading of the samples on the gel.

FIG. 15B depicts one proposed Caspase 9 protein structure.

FIG. 16A provides exemplary Western immunoblot gel electrophoresis data showing Caspase 3 activation in cell lysates of adherent DU 145 cells incubated with Ac-PHSCN-NH₂ peptide probed with a monoclonal antibody specific for the Asp¹⁷⁵ epitope of Caspase 3. Upper Gel: Cell Lysate; D1-D7=Incubation Day. C6=Control Culture tested on Day 6. Lower Gel: Actin Internal Standard.

FIG. 16B depicts one proposed Caspase 3 protein structure.

FIG. 17 provides exemplary Western immunoblot gel electrophoresis data showing Caspase 6 activation in cell lysates of adherent DU 145 cells incubated with Ac-PHSCN-NH₂ peptide. Upper Gel: Cell Lysate; D1-D7=Incubation Day. C6=Control Culture tested on Day 6. Lower Gel: Actin Internal Standard (used as a loading control).

FIG. 18 provides exemplary data showing induction of the cytokeratin 18 epitope, an epitope specific for cells in the early stages of apoptosis, in adherent DU 145 cells incubated for five (5) days with the Ac-PHSCN-NH₂ peptide, at a concentration of 200 μg per ml per 20,000 cells. X-Axis: Crosshatched Bar; Ac-PHSCN-NH₂ Treated. Open Bar: Untreated Control. Y-Axis: Mean Peroxidase Grains Per Cell.

FIG. 19 provides exemplary Western immunoblot gel electrophoresis data showing Ac-PHSCN-NH₂ (1 μg/ml) inhibition of 10% fetal calf serum (FCS) induced FAK phosphorylation in adherent DU 145 cells. Immunoblots were generated with a Y³⁹⁷ anti-FAK monoclonal antibody. Lanes: SF=Serum Free Control. 15′-360′=Times Of Incubation (minutes). hc=Heavy Chain.

FIG. 20 provides exemplary Western immunoblot gel electrophoresis data showing Ac-PHSCN-NH₂ (1 μg/ml) inhibition of 10% fetal calf serum (FCS) induced Akt phosphorlyation in adherent DU 145 cells. Immunoblots were generated with an S⁴⁷³ anti-Akt monoclonal antibody. Lanes: SF=Serum Free Control. 15′-360′=Times Of Incubation (minutes). hc=Heavy Chain.

FIG. 21 provides exemplary data showing fluoroscein labeled anti-biotin antibody detection of Ac-PHSCNGGK(biotin)-NH₂ tissue binding (Green). Photomicrograph A: Ac-PHSCNGGK-(biotin) binding To Tumor Cells. Photomicrograph B: Lack of Ac-PHSCNGGK-(biotin) Binding To Non-Tumor Cells. Cell nuclei were stained with 4′,6′-diamidino-2-phenylindole (DAPI).

FIG. 22 provides exemplary data showing: Photomicrograph A: Rhodamine labeled anti-biotin antibody detection of Ac-PHSCNGGK(biotin)-NH₂ tissue binding (Red). Photomicrograph B: Cell Nuclei detected using DAPI (Blue). Photomicrograph C: Endothelial cells detected using fluoroscein labeled anti-CD31 antibody (Green). Photomicrograph D: Superimposition of DAPI and fluoroscein labeled anti-CD31 antibody endothelial cell data (Red/Blue/Green).

FIG. 23 depicts one possible embodiment of an 8-substituted Ac-PHSCN dendrimers.

FIG. 24 provides exemplary data showing dose response curves for inhibiting serum-induced invasion of sea urchin embryo basement membranes by DU 145 prostate cancer cells. Triangles: 8-substituted Ac-PHSCN dendrimers. Open circles: Ac-PHSCN-NH₂ monomer. X Axis: Log peptide concentration (ng/ml). Y Axis: Percentage of invading DU145 cells. Mean invasion percentages are shown with their first standard deviations.

FIG. 25 provides an exemplary photomicrograph of normal DU 145 cells at 200×. Note the lack of cytoplasmic granules outside of the cells.

FIG. 26 provides an exemplary photomicrograph of normal DU 145 cells at 630×. Note the lack of cytoplasmic granules outside of the cells.

FIG. 27 provides an exemplary photomicrograph of 8-substituted Ac-PHSCN dendrimer treated DU 145 cells at 200×. Note that there are many cytoplasmic granules present outside of the cells.

FIG. 28 provides an exemplary photomicrograph of 8-substituted Ac-PHSCN dendrimer treated DU 145 cells at 200×. Note that there are many cytoplasmic granules present outside of the cells.

FIG. 29 provides an exemplary photomicrograph of 8-substituted Ac-PHSCN dendrimer treated DU 145 cells at 630×. Note that there are many cytoplasmic granules present outside of the cells.

FIG. 30 provides an exemplary photomicrograph of 8-substituted Ac-PHSCN dendrimer treated DU 145 cells at 400×. Note that there are many cytoplasmic granules present outside of the cells.

FIG. 31 presents exemplary data demonstrating the greater potency of one embodiment of an Ac-PHSCN dendrimer versus the Ac-PHSCN-NH₂ monomer peptide to inhibit the growth of DU145 prostate cancer cells in culture. X Axis: Incubation Day. Y Axis: Mean number of DU145 cells. Open Circles: Untreated control. Triangles: 8-substituted Ac-PHSCN dendrimer. Squares: Ac-PHSCN-NH₂ monomer peptide.

FIG. 32 presents exemplary data demonstrating the dose response relationship of one embodiment of an Ac-PHSCN dendrimer to inhibit the growth of DU145 prostate cancer cells in culture. X Axis: Incubation Day. Y Axis: Mean number of DU145 cells. Open Circles Untreated control. Triangles: 6 μg/ml. Squares: 20 μg/ml. Crosses: 60 μg/ml.

FIG. 33 presents exemplary data demonstrating the greater potency of one embodiment of an Ac-PHSCN dendrimer versus the Ac-PHSCN-NH₂ monomer peptide to inhibit in vivo MATLyLu tumor growth in intravenously injected Copenhagen rats (5 mg/kg; thrice weekly). Y Axis: Tumor Diameter (millimeters); X Axis: Open Bar: Untreated controls. Crosshatched Bar: Ac-PHSCN-NH₂ treated. Stippled Bar: 8-substituted Ac-PHSCN dendrimer treated. Mean tumor diameters with their first standard deviations are shown.

FIG. 34 presents exemplary data demonstrating the greater potency of one embodiment of an Ac-PHSCN dendrimer versus the Ac-PHSCN-NH₂ monomer peptide to inhibit the serum-induced invasion of BxPC-3 pancreatic cancer cells into a sea urchin embryo basement membrane substrate. Triangles: 8-substituted Ac-PHSCN dendrimers. Open circles: Ac-PHSCN-NH₂ monomer. X Axis: Log peptide concentration (ng/ml). Y Axis: Percentage of Invading BxPC-3 cells. Mean invasion percentages, with their first standard deviations, are shown.

FIG. 35 shows one possible biochemical pathway for apoptosis.

### DEFINITIONS

The term “drug” as used herein, refers to any medicinal substance used in humans or other animals. Encompassed within this definition are compound analogs, naturally occurring, synthetic and recombinant pharmaceuticals, hormones, antimicrobials, neurotransmitters, etc.

The term “inducing agent” refers to any compound or molecule which is capable of causing (directly or indirectly) the invasion of cells in a substrate. “Inducing agents” include, but are not limited to, PHSRN-containing (SEQ ID NO:1) peptides and related peptides (see below).

The term “receptors” refers to structures expressed by cells and which recognize binding molecules (e.g., ligands).

The term “antagonist” refers to molecules or compounds which inhibit the action of a “native” or “natural” compound (such as fibronectin). Antagonists may or may not be homologous to these natural compounds in respect to conformation, charge or other characteristics. Thus, antagonists may be recognized by the same or different receptors that are recognized by the natural compound. “Antagonists” include, but are not limited to, PHSCN-containing (SEQ ID NO:86) peptides and related peptides (see below).

The term “host cell” or “cell” refers to any cell which is used in any of the screening assays of the present invention. “Host cell” or “cell” also refers to any cell which either naturally expresses particular receptors of interest or is genetically altered so as to produce these normal or mutated receptors.

The term “chemotherapeutic agent” refers to molecules or compounds which inhibit the growth or metastasis of tumors. “Chemotherapeutics” include, but are not limited to, PHSCN-containing (SEQ ID NO:86) peptides and related peptides (see below).

As noted above, the present invention contemplates both the D and L isomers of cysteine which are identified collectively as “C”.

The present invention also contemplates homo-cysteine, which is identified as “hC”.

The term “dendrimer”, as used herein, refers to any macromolecule derived from a branches-upon-branches structural motif. Dendrimers are well defined, highly-branched macromolecules that radiate from a simple organic molecule as a core and may be synthesized through a stepwise, repetitive reaction sequence that guarantees complete shells for each generation leading theoretically to products that are unimolecular and monodisperse. The branching of the repeating-molecule polymer chains provides functional sites on which to attach substituents (i.e., for example, peptides, chemotherapeutic agents, or pharmaceutical compounds). One such dendrimer comprises a repeating polylysine molecule, however, other repeating molecules based on styrene or amido amines are equally useful.

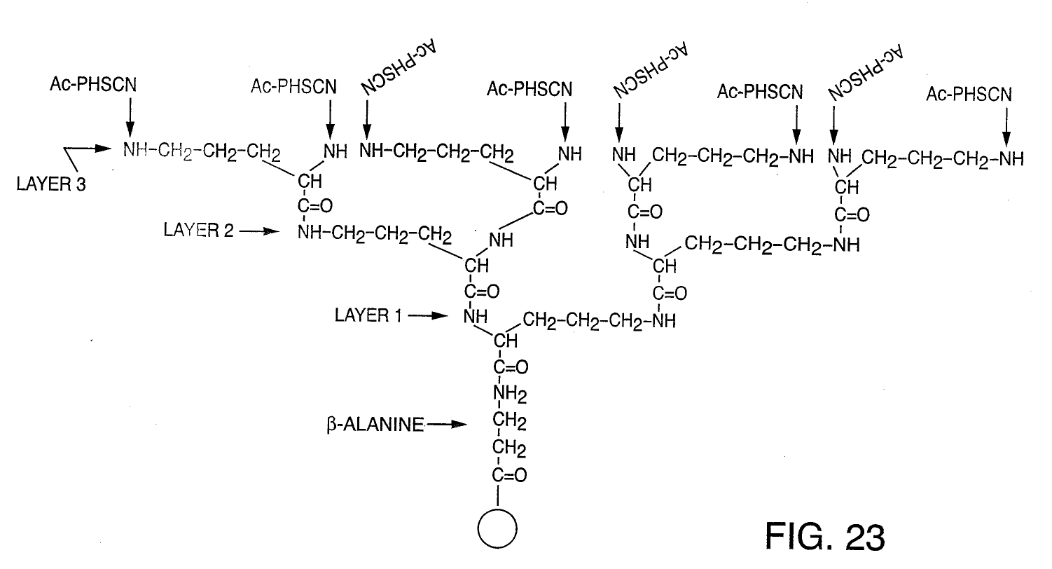

The term “branches” or “branching”, as used herein, refers to any repeating-molecule chain that alters the longitudinal axis angle of a dendrimer (i.e., for example, Levels 1-3 in FIG. 23).

The term “peptide”, as used herein, refers to any amino acid sequence comprising at least two amino acids.

The term “substituted”, as used herein, refers to any compound where at least one chemical moiety that has been replaced with a different chemical moiety. For example, an amine group comprising at least one hydrogen may be substituted with a peptide (i.e., for example, a PHSCN comprising peptide). Specifically, a polylysine dendrimer may comprise attached peptides, thereby creating a peptide-substituted dendrimer.

The term “attach”, “attachment”, “attached”, or “attaching”, as used herein, refers to any physical relationship between molecules that results in forming a stable complex. The relationship may be mediated by physico-chemical interactions including, but not limited to, ionic attraction. hydrogen bonding, covalent bonding, Van der Waals forces or hydrophobic attraction.

The term “receptor”, as used herein, refers to any structure which recognizes a binding molecule (e.g., a ligand). For example, a receptor may reside on a cell surface which recognizes a neurotransmitter.

The terms “protein”, “peptide”, or “polypeptide”, as used herein, refer to any compound comprising amino acids joined via peptide bonds and are used interchangeably. A “protein”, “peptide”, or “polypeptide” amino acid sequence may also comprise post-translational modifications.

The term “amino acid sequence”, as used herein, refers to the primary (i.e., linear) structure of a protein, peptide, or polypeptide.

The term “antibody”, as used herein, refers any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Antibodies may be polyclonal or monoclonal. Antibodies are generated by many methods known in the art, such as, but not limited to, host immunization, peptide combinatorial chemistry, or vector-mediated protein expression in cell culture.

The term “blood vessels”, as used herein, refers to any cardiovascular tissue comprising a vein or artery or capillary. It is known that some blood vessels comprise a variety of cells that associate with tumors (i.e., for example, endothelial cells). Such “tumor-associated blood vessels” are believed to provide nutrients, oxygen, and other required compounds to support tumor cell growth and maintenance.

The term “lymphatic vessels”, as used herein, refers to any vascular tissue comprising a vessel specialized to carry lymph.

The term “endothelial cells”, as used herein, refers to any cell that provides a lining for a bodily organ comprising a lumen (i.e., for example, blood vessels, intestines, lymphatic vessels or ducts etc.). Usually, endothelial cells provide physical and chemical protection as well a selective absorption of nutrients or other metabolically active compounds.

The term “tumor cell”, as used herein, refers to any mass of cells that exhibits uncontrolled growth patterns. Tumor cells may be derived from any tissue within an organism (i.e., for example, a prostate tumor cell).

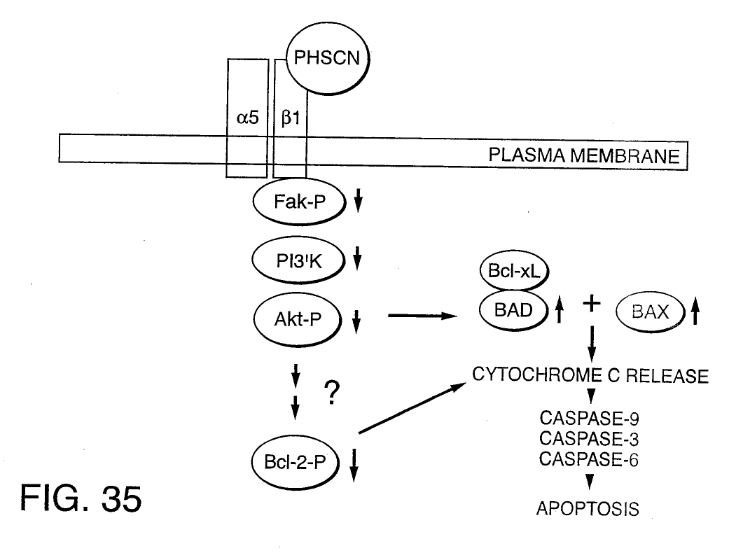

The term “apoptosis”, as used herein, refers to programmed cell death mediated by a biochemical pathway (i.e., for example, the focal adhesion kinase (FAK)/PI3′K/protein kinase B (Akt) biochemical pathway) (See FIG. 35).

The term “invasion”, as used herein, refers to the migration of tumor cells through tissues as they enter or leave the blood or lymphatic circulation.

The term “patient”, as used herein, refers to any living organism, preferably a mammal (i.e., for example, human or non-human), that may benefit from the administration of compositions contemplated herein. A patient may include either adults or juveniles (i.e., for example, children).

The term “necrosis”, as used herein, refers to any intra- or extracellular morphological changes indicative of cell death and caused by the progressive degradative action of enzymes in such a manner that it may affect groups of cells or parts of a bodily structure or an organ. Cellular necrosis characteristics may include, but is not limited to, rapid membrane lipid peroxidation, blebbing, and membrane breakdown.

### DESCRIPTION OF THE INVENTION

The present invention generally relates to the treatment of cancer, and more specifically, to the testing of cancer cells for their ability to invade tissues and cause metastases, and to the identification and use of drugs to inhibit tumor invasion and growth. As a prelude to metastasis, it is believed that cancer cells proteolytically alter basement membranes underlying epithelia or the endothelial linings of blood and lymphatic vessels, invade through the defects created by proteolysis, and enter the circulatory or lymphatic systems to colonize distant sites. During this process, the secretion of proteolytic enzymes is coupled with increased cellular motility and altered adhesion. After their colonization of distant sites, metastasizing tumor cells proliferate to establish metastatic nodules.

As noted above, chemotherapeutic agents are currently employed to reduce the unrestricted growth of cancer cells, either prior to surgical removal of the tumor (neoadjuvant therapy) or after surgery (adjuvant therapy). However, none of these methods has proved curative once metastasis has occurred. Since unrestricted invasive behavior is also a hallmark of metastatic tumor cells, methods for directly inhibiting tumor cell invasion and metastasis are needed.

### A. Assays for Testing Tumor Invasion

Discovering how to inhibit the invasive behavior of tumor cells to intervene in the metastatic cascade first requires the development of assays with which to test tumor cell invasion in vitro. Two assay systems are contemplated for use in the method of the present invention to test the tumor cell invasion.

1. Fibronectin-Depleted Substrates

In one assay system, the present invention contemplates using fibronectin-depleted substrates. These are substrates that originally contain fibronectin that are treated according to the methods of the present invention (see below) to remove fibronectin. It is not intended that the present invention be limited by the nature of the original substrate; such fibronectin-containing substrates suitable for treatment and depletion include: i) complex substrates containing a variety of extracellular proteins and ii) less complex substrates containing fibronectin along with one or two other proteins (e.g., collagen, laminin, etc.).

It is also not intended that the present invention be limited by the precise amount of fibronectin remaining after the substrate has been treated. In other words, while the methods of the present invention remove fibronectin, and in some embodiments, remove substantially all fibronectin, it is within the meaning of the term “fibronectin-depleted” substrate that a small amount of fibronectin remain in the substrate.

In one embodiment, the present invention contemplates using an extracellular matrix available commercially. For example, the present invention contemplates treating basement membrane matrices such as ECM GEL, a matrix from mouse sarcoma (commercially available from Sigma, St. Louis, Mo.). However, it is not intended that the present invention be limited by the particular fibronectin-containing substrate. For example, other commercially available substrates are contemplated, such as the commonly used substrate Matrigel (available from Becton Dickinson Labware, Catalog #40234); Matrigel can be treated appropriately according to the methods of the present invention so as to render it “fibronectin-depleted” (see below). Untreated Matrigel (and similar substrates) have been used to demonstrate the importance of proteases and motility factors in the invasion and metastasis of many tumors. However, these invasion substrates are not available as serum-free substrates; thus, the regulation of tumor cell invasive behavior by serum components, such as plasma fibronectin, is a complicating factor with untreated Matrigel.

Consequently, the present invention contemplates a fibronectin-free substrate. In this embodiment, Matrigel is treated so that it is substantially fibronectin-free. The preparation of fibronectin-free Matrigel involves “panning” the Matrigel substrate on gelatin as well as “panning” the substrate on anti-fibronectin antibody (anti-human fibronectin IgG is available commercially, such as antibody from Promega Corporation, Madison, Wis.).

2. Naturally Occurring Fibronectin-Free Substrates

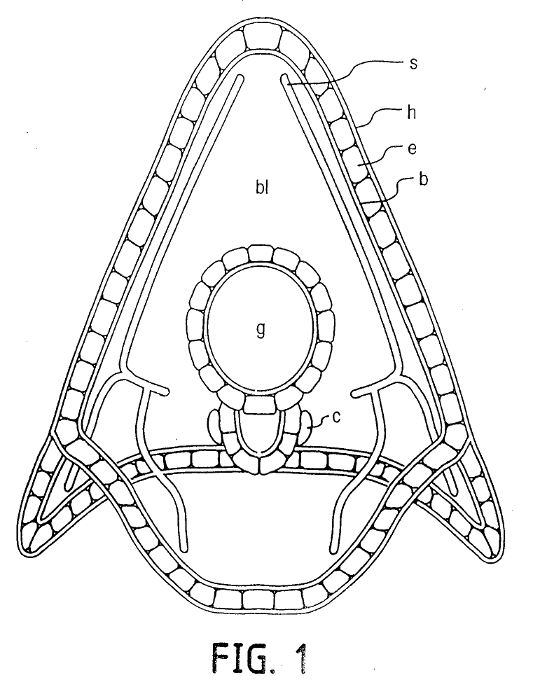

In another embodiment, the present invention contemplates substrates that are naturally free of fibronectin; such a source provides, for example, basement membranes permeable to select types of normally invasive cells, such membranes being naturally serum-free. In one embodiment, the present invention contemplates sea urchins as a source of such membranes. In this regard, the ectoderm of sea urchin embryos is one cell thick, and secretes an underlying basement membrane (see FIG. 1) very similar to that of mammals. These embryos contain no circulatory or lymphatic systems; and thus, their basement membranes are serum-free. In embryos, the subectodermal basement membrane functions simultaneously as a migration substrate for several, specific mesenchymal cell types while it functions as an invasion substrate for others. Sea urchin embryo basement membranes (SU-ECM) can be prepared by mild detergent treatment. Livant et al., Cancer Research 55:5085 (1995). Such procedures are described in the Experimental section below.

Regardless of which of the two types of substrates are employed, the invasion substrates of the present invention are easy to prepare and give rapid, highly consistent results with a variety of cells, including: a) cell lines from: i) primary and metastatic tumors, and ii) normal epithelial tissues; as well as b) cells from primary tissue samples of both tumors, their surrounding normal tissues, and neonatal melanocytes, fibroblasts, and keratinocytes from circumcised tissue.

In one embodiment, the present invention contemplates a method of evaluating human cancer comprising: a) providing: i) a human cancer patient (such as a patient with breast cancer or prostate cancer), ii) a fibronectin-free substrate (for example, a fibronectin-depleted substrate) and iii) one or more invasion-inducing agents (discussed below); b) obtaining cancer cells from said patient (such as from a biopsy); c) contacting said cells ex vivo (i.e., outside the body) with said fibronectin-free substrate and said one or more invasion-inducing agents; and d) measuring the extent of cancer cell invasion of said substrate. Preferably the cancer cells are cultured in serum-free culture media so as to avoid introducing complicating factors.

3. Inducing Agents

It is not intended that the present invention be limited by the nature of the agent that causes or induces cells to invade the fibronectin-free substrates of the present invention. Such agents can be identified functionally by simply adding them to the cell culture and measuring the extent of invasion.

In one embodiment, the invasion-inducing agent comprises a peptide derived from fibronectin. In a preferred embodiment, the invasion inducing agent is intact fibronectin.

While not limited to any mechanism, it is believed that cells exposed to invasion-inducing agents in this manner are potentially rendered capable of invading the substrate. Again, while not limited to any mechanism, it is believed that the invasion inducing agent comprising the sequence PHSRN (SEQ ID NO:1) binds to the α5β1 receptor on the cancer cell and thereby induces invasion of the substrate. In this regard, the present invention provides a method of treating cells comprising: a) providing: i) cells expressing the α5β1 receptor, ii) a fibronectin-free substrate, and iii) one or more invasion-inducing agents; b) culturing said cells in serum-free culture'media on said substrate in the presence of said invasion-inducing agents; and d) measuring the extent of cell invasion of said substrate. In one embodiment, the cells are normal epithelial cells or fibroblasts. In another embodiment, the cells are human cancer cells.

### B. Drug Screening Assays

The present invention also contemplates a screening assay for identifying drugs that inhibit tumor invasion. The present invention contemplates a screening assay (in the presence and absence of serum) utilizing the binding activity of fibronectin-derived peptides. In one embodiment, an inducible tumor cell line is placed in tissue culture on a fibronectin-free substrate. The tumor cells will be induced (under ordinary conditions) by the fibronectin-derived peptide to invade the substrate.

In one embodiment, the invasion-inducing agent comprises a peptide derived from fibronectin. In a preferred embodiment, said peptide comprises the sequence PHSRN (SEQ ID NO:1). Of course, the peptide may be larger than five amino acids; indeed, the peptide fragment of fibronectin may contain hundreds of additional residues (e.g., five hundred amino acids). One such larger peptide is set forth in U.S. Pat. No. 5,492,890 (hereby incorporated by reference). In one embodiment, the PHSRN-containing (SEQ ID NO:1) peptide is less than one hundred amino acids in length and lacks the RGD (SEQ ID NO:81) sequence characteristic of fibronectin. A variety of PHSRN-containing (SEQ ID NO:1) peptides are contemplated, including the PHSRN (SEQ ID NO:1) peptide itself and related peptides where additional amino acids are added to the carboxyl terminus, including (but not limited to) peptides comprising the sequence: 1) PHSRN (SEQ ID NO:1), 2) PHSRNS (SEQ ID NO:3), 3) PHSRNSI (SEQ ID NO:4), 4) PHSRNSIT (SEQ ID NO:5), 5) PHSRNSITL (SEQ ID NO:6), 6) PHSRNSITLT (SEQ ID NO:7), 7) PHS-RNSITLTN (SEQ ID NO:8), 8) PHSRNSITLTNL (SEQ ID NO:9), 9) PHSRNSITL-TNLT (SEQ ID NO:10), 10) PHSRNSITLTNLTP (SEQ ID NO:11), and 11) PHSRN-SITLTNLTPG (SEQ ID NO:12). Alternatively, PHSRN-containing (SEQ ID NO:1) peptides are contemplated where amino acids are added to the amino terminus, including (but not limited to) peptides comprising the sequence: 1) PEHFSGRPREDRVPHSRN (SEQ ID NO:13), 2) EHFSGRPREDRVPHSRN (SEQ ID NO:14), 3) HFSGRPREDR-VPHSRN (SEQ ID NO:15), 4) FSGRPREDRVPHSRN (SEQ ID NO:16), 5) SGRPRED-RVPHSRN (SEQ ID NO:17), 6) GRPREDRVPHSRN (SEQ ID NO:18), 7) RPRED-RVPHSRN (SEQ ID NO:19), 8) PREDRVPHSRN (SEQ ID NO:20), 9) REDRVPHSRN (SEQ ID NO:21), 10) EDRVPHSRN (SEQ ID NO:22), 11) DRVPHSRN (SEQ ID NO:23), 12) RVPHSRN (SEQ ID NO:24), and 13) VPHSRN (SEQ ID NO:25). Finally, the present invention contemplates PHSRN-containing (SEQ ID NO:1) peptides where amino acids are added to both the amino and carboxyl termini, including, but not limited to, peptides comprising the sequence PEHFSGRPREDRVPHSRNSITLTNLTPG (SEQ ID NO:26), as well as peptides comprising portions or fragments of the PHSRN-containing (SEQ ID NO:1) sequence PEHFSGRPREDRVPHSRNSITLTNLTPG (SEQ ID NO:26).

Peptides containing variations on the PHSRN (SEQ ID NO:1) motif are contemplated. For example, the present invention also contemplates PPSRN-containing (SEQ ID NO:27) peptides for use in the above-named assays. Such peptides may vary in length in the manner described above for PHSRN-containing (SEQ ID NO:1) peptides. Alternatively, PPSRN (SEQ ID NO:27) may be used as a peptide of five amino acids.

Similarly, peptides comprising the sequence -HHSRN- (SEQ ID NO:28), 1-HPSRN-SEQ ID NO:29), -PHTRN- (SEQ ID NO:30)-HHTRN- (SEQ ID NO:31), -HPTRN- (SEQ ID NO:32), -PHSNN- (SEQ ID NO:33), -HHSNN- (SEQ ID NO:34), -HPSNN- (SEQ ID NO:35), -PHTNN- (SEQ ID NO:36), -HHTNN- (SEQ ID NO:37), -HPTNN- (SEQ ID NO:38), -PHSKN- (SEQ ID NO:39), -HHSKN- (SEQ ID NO:40), -HPSKN- (SEQ ID NO:41), -PHTKN- (SEQ ID NO:42), -HHTKN- (SEQ ID NO:43), -HPTKN- SEQ ID NO:44), -PHSRR- (SEQ ID NO:45), -HHSRR- (SEQ ID NO:46), -HPSRR- (SEQ ID NO:47), -PHTRR- (SEQ ID NO:48), -HHTRR- (SEQ ID NO:49), -HPTRR- (SEQ ID NO:50), -PHSNR- (SEQ ID NO:51), -HHSNR- (SEQ ID NO:52), -HPSNR- (SEQ ID NO:53), -PHTNR- (SEQ ID NO:54), -HHTNR- (SEQ ID NO:55), -HPTNR- (SEQ ID NO:56), -PHSKR- (SEQ ID NO:57), -HHSKR- (SEQ ID NO:58), -HPSKR- (SEQ ID NO:59), -PHTKR- (SEQ ID NO:60), -HHTKR- (SEQ ID NO:61), -HPTKR- (SEQ ID NO:62), -PHSRK- (SEQ ID NO:63), -HHSRK- (SEQ ID NO:64), -HPSRK- (SEQ ID NO:65), -PHTRK- (SEQ ID NO:66), -HHTRK- (SEQ ID NO:67), -HPTRK- (SEQ ID NO:68), -PHSNK- (SEQ ID NO:69), -HHSNK- (SEQ ID NO:70), -HPSNK- (SEQ ID NO:71), -PHTNK- (SEQ ID NO:72), -HHTNK- (SEQ ID NO:73), -HPTNK- (SEQ ID NO:74), -PHSKK- (SEQ ID NO:75), -HHSKK- (SEQ ID NO:76), -HPSKK- (SEQ ID NO:77), -PHTKK- (SEQ ID NO:78), -HHTKK- (SEQ ID NO:79), or -HPTKK- (SEQ ID NO:80) are contemplated by the present invention. Such peptides can be used as five amino acid peptides or can be part of a longer peptide (in the manner set forth above for PHSRN-containing (SEQ ID NO:1) peptides).

In another embodiment, the present invention contemplates an inducing agent comprising the amino acid sequence X₁X₂X₃X₄X₅ (SEQ ID NO:93), wherein X₁ is an amino acid selected from the group consisting of proline, glycine, valine, histidine, isoleucine, phenylalanine, tyrosine, and tryptophan, and X₂ is an amino acid selected from the group consisting of histidine, proline, tyrosine, asparagine, glutamine, arginine, lysine, phenylalanine, and tryptophan, and X₃ is an amino acid selected from the group consisting of serine, threonine, alanine, tyrosine, leucine, histidine, asparagine, and glutamine, and X₄ is an amino acid selected from the group consisting of arginine, lysine, and histidine, and X₅ is an amino acid selected from the group consisting of asparagine, glutamine, serine, threonine, histidine, and tyrosine.

In this drug screening assay, candidate drug inhibitors are added to the tissue culture (this can be done individually or in mixtures). Where the inducible tumor cell is found to be inhibited from invading the substrate, a drug inhibitor is indicated (see Examples section below using the PHSCN (SEQ ID NO:86) peptide).

It is not intended that the present invention be limited by the nature of the drugs screened in the screening assay of the present invention. A variety of drugs, including peptides and non-peptide mimetics, are contemplated.

It is also not intended that the present invention be limited by the particular tumor cells used for drug testing. A variety of tumor cells (for both positive and negative controls) are contemplated (including but not limited to the cells set forth in Table 1 below).

### C. Invasion-Inducing Agents and Antagonists

While an understanding of the mechanisms involved in metastatic cancer is not necessary to the successful practice of the present invention, it is believed that tumor cell invasion of basement membranes occurs at several points in the metastatic cascade: (1) when epithelial tumor cells (such as those of breast and prostate cancers) leave the epithelium and enter the stroma, (2) when tumor cells enter the circulatory or lymphatic systems, and (3) when tumor cells leave the circulatory or lymphatic systems to invade distant sites. Thus, intervention in the induction of tumor cell invasiveness by using a PHSRN (SEQ ID NO:1) antagonist, such as the PHSCN (SEQ ID NO:86) peptide, to block tumor cell receptors for this sequence is contemplated as a method for decreasing the rate of metastasis.

One advantage of this strategy is that leukocytes are the only normal cells known to invade tissues to carry out their functions, and relatively few leukocytes are invasive at a given time. Thus, relatively small doses of an anti-invasion antagonist which blocks the binding of PHSRN (SEQ ID NO:1) to its receptor are required. Also, other than some immunodepression, there should be relatively few side effects associated with anti-metastatic treatment using compounds designed to block the induction of invasion. The lack of debilitating side effects expected from anti-invasive therapy means that using it in combination with anti-proliferative agents would be uncomplicated, and that it could be used prior to surgery or even prophylactically to block tumor cell invasion and metastasis.

The IKVAV (SEQ ID NO:2) sequence of laminin, a prevalent insoluble protein of the extracellular matrix, is known to stimulate liver colonization by metastatic human colon cancer cells in athymic mice. Bresalier et al., Cancer Research 55:2476 (1995). Since IKVAV (SEQ ID NO:2), like PHSRN (SEQ ID NO:1), contains a basic amino acid (K) which, by virtue of its positive charge, might also function to displace a divalent cation from its integrin receptor and stimulate invasion, the present invention contemplates applying the strategy of developing anti-invasion antagonists to the IKVAV (SEQ ID NO:2) sequence of laminin.

TABLE 1
Designation And Origin Of Human Cell Lines And Strains¹

| Origin | Cell Lines or Strains |
|---|---|
| Colonic | SW1116, HCT116, SKCO-1, HT-29, KM12C, |
| carcinoma | KM12SM, KM12L4, SW480 |
| Pancreatic | BxPC-3, AsPC-1, Capan-2, MIA PaCa-2, Hs766T |
| carcinoma | |
| Colon adenoma | VaCo 235 |
| Lung carcinoma | A549 |
| Prostate | PC-3, DU-145 |
| carcinoma | |
| Breast carcinoma | 009P, 013T, SUM-52 PE |
| Lymphoma | Daudi, Raji |
| Breast epithelium | 006FA |
| Diploid fibroblast | HCS (human corneal stroma), MRC-5 |

¹The SW1116, HT-29, SW480, Raji lymphoblastoid cells, and the pancreatic lines are obtained from the American Type Culture Collection

1. Antagonists

It is not intended that the present invention be limited by the nature of the agent that inhibits tumor invasiveness. A variety of anti-invasive chemotherapeutics are contemplated to antagonize the invasion-promoting activity of the PHSRN (SEQ ID NO:1) sequence.

In the preferred embodiment, the anti-invasive agent is a peptide with the amino acid sequence PHSCN (SEQ ID NO:86). In another embodiment, the anti-invasive agent is a peptide which has an amino acid sequence comprising a sequence selected from the group consisting of CHSRN (SEQ ID NO:87), PCSRN (SEQ ID NO:88), PHCRN (SEQ ID NO:89), and PHSRC (SEQ ID NO:90). In another embodiment, the anti-invasive agent is a peptide which has an amino acid sequence comprising PHSXN (SEQ ID NO:91), where X is an amino acid selected from the group consisting of homo-cysteine, the D-isomer of cysteine, histidine, or penicillamine.

The present invention also contemplates an anti-invasive agent comprising the amino acid sequence X₁HSX₂N (SEQ ID NO:92), wherein X₁ is either proline, histidine, or not an amino acid, and X₂ is an amino acid selected from the group consisting of the L-isomer of cysteine, the D-isomer of cysteine, homo-cysteine, histidine, or penicillamine. In another embodiment, the present invention contemplates an anti-invasive agent comprising the amino acid sequence X₁X₂X₃X₄X₅ (SEQ ID NO:93), wherein X₁ is an amino acid selected from the group consisting of proline, glycine, valine, histidine, isoleucine, phenylalanine, tyrosine, and tryptophan, and X₂ is an amino acid selected from the group consisting of histidine, proline, tyrosine, asparagine, glutamine, arginine, lysine, phenylalanine, and tryptophan, and X₃ is an amino acid selected from the group consisting of serine, threonine, alanine, tyrosine, leucine, histidine, asparagine, and glutamine, and X₄ is an amino acid selected from the group consisting of cysteine, homo-cysteine, penicillamine, histidine, tyrosine, asparagine, glutamine, and methionine, and X₅ is an amino acid selected from the group consisting of asparagine, glutamine, serine, threonine, histidine, and tyrosine. In the preferred embodiment the peptide is PHSCN (SEQ ID NO:86), where the cysteine is the L-isomer.

Similarly, peptides comprising the sequence -PSCN- (SEQ ID NO:102), 1-HSCN- (SEQ ID NO:96), -PSCN- (SEQ ID NO:102), -HTCN- (SEQ ID NO:99), -PTCN- (SEQ ID NO:105), -PSCN- (SEQ ID NO:102), -HTCN- (SEQ ID NO:99), -HTCN- (SEQ ID NO:99), -PTCN- (SEQ ID NO:105), -HSCN- (SEQ ID NO:96), -PSCN- (SEQ ID NO:102), -HTCN- (SEQ ID NO:99), -HTCN- (SEQ ID NO:99), -PTCN- (SEQ ID NO:105), -HSCR- (SEQ ID NO:97), -HSCR- (SEQ ID NO:97), -PSCR- (SEQ ID NO:103), -HTCR- (SEQ ID NO:100), -HTCR- (SEQ ID NO:100), -PTCR- (SEQ ID NO:106), -HSCR- (SEQ ID NO:97), -HSCR- (SEQ ID NO:97), -PSCR- (SEQ ID NO:103), -HTCR- (SEQ ID NO:100), -HTCR- (SEQ ID NO:100), -PTCR- (SEQ ID NO:106), -HSCR-(SEQ ID NO:97), -HSCR- (SEQ ID NO:97), -PSCR- (SEQ ID NO:103), -HTCR- (SEQ ID NO:100), -HTCR- (SEQ ID NO:100), -PTCR- (SEQ ID NO:106), -HSCK- (SEQ ID NO:95), -HSCK- (SEQ ID NO:95), -PSCK- (SEQ ID NO:101), -HTCK- (SEQ ID NO:98), -HTCK- (SEQ ID NO:98), -PTCK- (SEQ ID NO:104), -HSCK- (SEQ ID NO:95), -HSCK- (SEQ ID NO:95), -PSCK- (SEQ ID NO:101), -HTCK- (SEQ ID NO:98), -HTCK- (SEQ ID NO:98), -PTCK- (SEQ ID NO:104), -HSCK- (SEQ ID NO:95), -HSCK- (SEQ ID NO:95), -PSCK- (SEQ ID NO:101), -HTCK- (SEQ ID NO:98), -HTCK- (SEQ ID NO:98), or -PTCK- (SEQ ID NO:104) are contemplated by the present invention.

It is further contemplated that, in some embodiments, the anti-invasive agents named above comprise the named amino acid sequence and additional amino acids added to the amino terminus, the carboxyl terminus, or both the amino and carboxyl termini as in the manner set forth above for the PHSRN (SEQ ID NO:1) containing peptides, e.g., PHSRNSIT (SEQ ID NO:5). In one embodiment, the anti-invasive agent is up to five hundred amino acids in length. It is also contemplated that, in some embodiments, the anti-invasive agents named above comprise a peptide with the amino terminus blocked by standard methods to prevent digestion by exopeptidases, for example by acetylation; and the carboxyl terminus blocked by standard methods to prevent digestion by exopeptidases, for example, by amidation.

In this regard, the present invention provides a method of treating cancer comprising: a) providing: i) a subject having cancer, and ii) a composition of matter comprising a peptide, peptide derivative, or peptide mimetic which inhibits the tumor invasion-promoting activity of a peptide comprising the amino acid sequence PHSRN (SEQ ID NO:1), and b) administering said composition to said subject. The present invention further contemplates using antagonists before and/or after surgical removal of the primary tumor. In one embodiment, the method comprises administering a PHSRN (SEQ ID NO:1) antagonist as adjunct therapy with additional chemotherapeutics.

While not limited to any mechanism, it is believed that these anti-invasive chemotherapeutic agents antagonize the invasion-promoting activity of the PHSRN (SEQ ID NO:1) sequence (e.g., of fibronectin) by blocking the binding of this sequence to its receptor on tumor cells. Again, while not limited to any mechanism, it is believed that the PHSRN (SEQ ID NO:1) sequence may promote invasion by acting to displace a divalent cation (Mg⁺², Ca⁺², or Mn⁺) in the α5β1 receptor on metastatic tumor cells, and the above named chemotherapeutic anti-invasive agents might act to inhibit this invasion by chelating one or more of these divalent cations.

In another embodiment, the present invention contemplates anti-invasion antagonists to the IKVAV (SEQ ID NO:2) sequence of laminin.

2. Designing Mimetics

Compounds mimicking the necessary conformation for recognition and docking to the receptor binding to the peptides of the present invention are contemplated as within the scope of this invention. For example, mimetics of PHSRN (SEQ ID NO:1) and PHSRN-antagonists (SEQ ID NO:1) are contemplated. A variety of designs for such mimetics are possible. For example, cyclic PHSRN (SEQ ID NO:1) and PHSCN (SEQ ID NO:86) containing peptides, in which the necessary conformation for binding is stabilized by nonpeptides, are specifically contemplated. U.S. Pat. No. 5,192,746 to Lobl, et al., U.S. Pat. No. 5,169,862 to Burke, Jr., et al., U.S. Pat. No. 5,539,085 to Bischoff, et al., U.S. Pat. No. 5,576,423 to Aversa, et al., U.S. Pat. No. 5,051,448 to Shashoua, and U.S. Pat. No. 5,559,103 to Gaeta, et al. (all hereby incorporated by reference) describe multiple methods for creating such compounds.

Synthesis of nonpeptide compounds that mimic peptide sequences is also known in the art, such as, nonpeptide antagonists that mimic the Arg-Gly-Asp sequence. Eldred et al., J. Med. Chem. 37:3882 (1994). Further elucidation of the synthesis of a series of such compounds is also described. Ku et al., J. Med. Chem. 38:9 (1995). Such nonpeptide compounds that mimic PHSRN (SEQ ID NO:1) and PHSRN-antagonists (SEQ ID NO:1) are specifically contemplated by the present invention.

The present invention also contemplates synthetic mimicking compounds that are multimeric compounds that repeat the relevant peptide sequences. In one embodiment of the present invention, it is contemplated that the relevant peptide sequence is Pro-His-Ser-Arg-Asn (SEQ ID NO:1); in another embodiment, the relevant peptide sequence is Pro-His-Ser-Cys-Asn (SEQ ID NO:86); in another embodiment, the relevant peptide sequence is Ile-Lys-Val-Ala-Val (SEQ ID NO:2). As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the α-amino group of the component containing the activated carboxyl group can be blocked with a tertbutyloxycarbonyl group. This protecting group can be subsequently removed by exposing the peptide to dilute acid, which leaves peptide bonds intact. With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the mimetics of the present invention are peptides having sequence homology to the above-described PHSRN (SEQ ID NO:1) sequences and PHSRN-antagonists (SEQ ID NO:1). One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant. W. R. Pearson and D. J. Lipman, Proc. Natl. Acad. Sci. (USA), 85:2444-2448 (1988); and D. J. Lipman and W. R. Pearson, Science, 227:1435-1441 (1985). In the present invention, synthetic polypeptides useful in tumor therapy and in blocking invasion are those peptides with statistically significant sequence homology and similarity (Z value of Lipman and Pearson algorithm in Monte Carlo analysis exceeding 6).

3. Antibody Inhibitors

The present invention contemplates all types of inhibitors of tumor invasion for use in both the assays and for therapeutic use. In one embodiment, the present invention contemplates antibody inhibitors. The antibodies may be monoclonal or polyclonal, but polyclonal antibodies are often more effective inhibitors. It is within the scope of this invention to include any second antibodies (monoclonal or polyclonal) directed to the first antibodies discussed above. Both the first and second antibodies may be used in the detection assays or a first antibody may be used with a commercially available anti-immunoglobulin antibody. An antibody as contemplated herein includes any antibody specific to any region of a peptide involved in the induction of tumor cell invasion. For example, the present invention contemplates antibodies reactive with PHSRN (SEQ ID NO:1) peptides (as well as the related peptides set forth above).

Both polyclonal and monoclonal antibodies are obtainable by immunization with peptides, as well as with enzymes or proteins, and all types are utilizable for immunoassays. The methods of obtaining both types of sera are well known in the art. Polyclonal sera are less preferred but are relatively easily prepared by injection of a suitable laboratory animal with an effective amount of the purified enzyme or protein, or antigenic parts thereof, collecting serum from the animal, and isolating specific sera by any of the known immunoadsorbent techniques. Although antibodies produced by this method are utilizable in virtually any type of immunoassay, they are generally less favored because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. See, for example, Douillard and Hoffman, Basic Facts About Hybridomas, in Compendium of Immunology Vol II, ed. by Schwartz, 1981; and Kohler et al., Nature 256: 495-499, (1975); or European Journal of Immunology 6:511-519 (1976).

Unlike preparation of polyclonal sera, the choice of animal is dependent on the availability of appropriate immortal lines capable of fusing with lymphocytes. Mouse and rat have been the animals of choice in hybridoma technology and are preferably used. Humans can also be utilized as sources for sensitized lymphocytes if appropriate immortalized human (or nonhuman) cell lines are available. For the purpose of the present invention, the animal of choice may be injected with an antigenic amount, for example, from about 0.1 mg to about 20 mg of the enzyme or protein or antigenic parts thereof. Usually the injecting material is emulsified in Freund's complete adjuvant. Boosting injections may also be required. The detection of antibody production can be carried out by testing the antisera with appropriately labelled antigen. Lymphocytes can be obtained by removing the spleen of lymph nodes of sensitized animals in a sterile fashion and carrying out fusion. Alternatively, lymphocytes can be stimulated or immunized in vitro. Reading et al., Journal of Immunological Methods 53: 261-291 (1982).

A number of cell lines suitable for fusion have been developed and the choice of any particular line for hybridization protocols is directed by any one of a number of criteria such as speed, uniformity of growth characteristics, deficiency of its metabolism for a component of the growth medium, and potential for good fusion frequency.

Intraspecies hybrids, particularly between like strains, work better than interspecies fusions. Several cell lines are available, including mutants selected for the loss of ability to secrete myeloma immunoglobulin.

Cell fusion can be induced either by virus, such as Epstein-Barr or Sendai virus, or polyethylene glycol. Polyethylene glycol (PEG) is the most efficacious agent for the fusion of mammalian somatic cells. PEG itself may be toxic for cells and various concentrations should be tested for effects on viability before attempting fusion. The molecular weight range of PEG may be varied from 1000 to 6000. It gives best results when diluted to from about 20% to about 70% (w/w) in saline or serum-free medium. Exposure to PEG at 37° C. for about 30 seconds is preferred in the present case, utilizing murine cells. Extremes of temperature (i.e., about 45° C.) are avoided, and preincubation of each component of the fusion system at 37° C. prior to fusion can be useful. The ratio between lymphocytes and malignant cells is optimized to avoid cell fusion among spleen cells and a range of from about 1:1 to about 1:10 is commonly used.

The successfully fused cells can be separated from the myeloma line by any technique known by the art. The most common and preferred method is to choose a malignant line which is Hypoxthanine Guanine Phosphoribosyl Transferase (HGPRT) deficient, which will not grow in an aminopterin-containing medium used to allow only growth of hybrids and which is generally composed of hypoxthanine 1×10⁻⁴M, aminopterin 1×10⁻⁵M, and thymidine 3×10⁻⁵M, commonly known as the HAT medium. The fusion mixture can be grown in the HAT-containing culture medium immediately after the fusion 24 hours later. The feeding schedules usually entail maintenance in HAT medium for two weeks and then feeding with either regular culture medium or hypoxthanine, thymidine-containing medium.

The growing colonies are then tested for the presence of antibodies that recognize the antigenic preparation. Detection of hybridoma antibodies can be performed using an assay where the antigen is bound to a solid support and allowed to react to hybridoma supernatants containing putative antibodies. The presence of antibodies may be detected by “sandwich” techniques using a variety of indicators. Most of the common methods are sufficiently sensitive for use in the range of antibody concentrations secreted during hybrid growth.

Cloning of hybrids can be carried out after 21-23 days of cell growth in selected medium. Cloning can be preformed by cell limiting dilution in fluid phase or by directly selecting single cells growing in semi-solid agarose. For limiting dilution, cell suspensions are diluted serially to yield a statistical probability of having only one cell per well. For the agarose technique, hybrids are seeded in a semi-solid upper layer, over a lower layer containing feeder cells. The colonies from the upper layer may be picked up and eventually transferred to wells.

Antibody-secreting hybrids can be grown in various tissue culture flasks, yielding supernatants with variable concentrations of antibodies. In order to obtain higher concentrations, hybrids may be transferred into animals to obtain inflammatory ascites. Antibody-containing ascites can be harvested 8-12 days after intraperitoneal injection. The ascites contain a higher concentration of antibodies but include both monoclonals and immunoglobulins from the inflammatory ascites. Antibody purification may then be achieved by, for example, affinity chromatography.

A wide range of immunoassay techniques are available for evaluating the antibodies of the present invention as can be seen by reference to U.S. Pat. Nos. 4,016,043; 4,424,279 and 4,018,653, hereby incorporated by reference. This, of course, includes both single-site and two-site, or “sandwich”, assays of the non-competitive types, as well as in the traditional competitive binding assays.

4. Administering Chemotherapeutics

It is contemplated that the antagonists of the present invention be administered systemically or locally to inhibit tumor cell invasion in cancer patients with locally advanced or metastatic cancers. They can be administered intravenously, intrathecally, intraperitoneally as well as orally. PHSRN (SEQ ID NO:1) antagonists (e.g., the PHSCN (SEQ ID NO:86) peptide), can be administered alone or in combination with anti-proliferative drugs in a neoadjuvant setting to reduce the metastatic load in the patient prior to surgery; or they can be administered after surgery. Since PHSRN (SEQ ID NO:1) antagonists may depress wound healing (because the PHSRN (SEQ ID NO:1) sequence also elicits fibroblast invasion as described below), it may be necessary to use PHSRN (SEQ ID NO:1) antagonists some time after surgery to remove the tumor.

Since few cells in the body must invade in order to function, PHSRN (SEQ ID NO:1) antagonists administered systemically are not likely to cause the debilitating side effects of cytotoxic chemotherapeutic agents. However, since they suppress invasion, they are likely to cause some immunodepression. Even so, at the appropriate dosage, PSHRN (SEQ ID NO:1) antagonists may be administered prophylactically. In any case, it is contemplated that they may be administered in combination with cytotoxic agents. The simultaneous selection against the two fatal attributes of metastatic cells, unrestricted proliferation and invasion, is contemplated as a very powerful therapeutic strategy.

Where combinations are contemplated, it is not intended that the present invention be limited by the particular nature of the combination. The present invention contemplates combinations as simple mixtures as well as chemical hybrids. An example of the latter is where the antagonist is covalently linked to a targeting carrier or to an active pharmaceutical. Covalent binding can be accomplished by any one of many commercially available crosslinking compounds.

It is not intended that the present invention be limited by the particular nature of the therapeutic preparation. For example, such compositions can be provided together with physiologically tolerable liquid, gel or solid carriers, diluents, adjuvants and excipients.

These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

Such compositions are typically prepared as liquid solutions or suspensions, or in solid forms. Oral formulations for cancer usually will include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%.

The compositions are also prepared as injectables, either as liquid solutions or suspensions; solid forms suitable for solution in, or suspension in, liquid prior to injection may also be prepared.

The antagonists of the present invention are often mixed with diluents or excipients which are physiological tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

5. Administering Anti-Thrombotics

In addition to using the PHSRN (SEQ ID NO:1) antagonists described above as anti-invasion chemotherapeutics, it is also contemplated that these antagonists be used as anti-thrombotics. This use of the PHSRN (SEQ ID NO:1) antagonists described above is based on the discovery that PHSCN (SEQ ID NO:86) peptide-treated blood appears in vivo to clot very slowly.

A number of anti-thrombotic agents are currently known which inhibit clot formation by preventing platelet integrins from binding fibrinogen or fibronectin. These anti-thrombotics, however, rely on competitive inhibition to prevent platelet integrins from binding to fibrinogen or fibronectin. In this manner, large doses of these agents are required to achieve the desired anti-thrombotic affect.

The present invention contemplates a more effective approach using PHSRN-antagonists (SEQ ID NO:1) such as PHSCN (SEQ ID NO:86). While the precise mechanism need not be known to practice the invention it has been shown that the platelet integrin, αIIbβ3, also binds the PHSRN (SEQ ID NO:1) sequence of plasma fibronectin. Thus, instead of utilizing competitive inhibition, the PHSRN-antagonists (SEQ ID NO:1) may directly inhibit platelet integrins from binding fibronectin and aggregating. Specifically, the PHSCN (SEQ ID NO:86) peptide, or other PHSRN-antagonists (SEQ ID NO:1), may directly inhibit early stages in clot formation by binding to the αIIbβ3 receptors on platelets. This prevents platelet integrins from binding fibronectin, a necessary part of platelet aggregation, thus inhibiting an integral step in the blood clotting cascade. In this manner, a comparatively small dose of the PHSCN (SEQ ID NO:86) peptide, or other PHSRN (SEQ ID NO:1) antagonist, is contemplated as effective anti-thrombotic agents.

6. Administering Wound Healing Agents

As noted above, it is contemplated that PHSRN (SEQ ID NO:1) antagonists may depress wound healing. This expectation is based on the discovery that PHSRN-containing (SEQ ID NO:1) peptides promote wound healing.

In this regard, it should be noted that the therapy of wounds, particularly those which are made difficult to heal by disease, has been attempted with a variety of purified growth factors or cytokines because these molecules can induce cellular proliferation or increase the motility of cells in wounds. Thus, if presented in the correct form and location at the right time, growth factors may greatly accelerate or enhance the healing of wounds by stimulating the growth of new tissue. Given the complexity and clinical variability of wounds, an obvious difficulty with the application of specific, purified growth factors or cytokines to wounded tissue, alone or in combination, is that their forms or specific distributions in the wound may not support their normal activities. Instead, the effectiveness of growth factors and cytokines in promoting the healing of wounded tissue may depend on their secretion by fibroblasts or macrophages.

The present invention contemplates a more effective approach; this approach involves methods that stimulate the invasion of the wound by the cells which synthesize the growth factors and cytokines active in stimulating wound repair, especially monocytes, macrophages, and fibroblasts. This strategy allows the cells in their normal in vivo setting to secrete the active factors. This approach has a number of advantages: (1) the temporal and spatial distributions of the factors are likely to be optimal because the normally active cells in their correct settings are secreting them; (2) all the appropriate factors are likely to be present in their active forms, irrespective of whether they have been identified or cloned; (3) the sequential effects of the factors in recruiting subsequent waves of cells involved in the healing process to the wound site are likely to be enhanced by the presence of more initiating cells in the wound.

The present invention is based on the discovery that the pure PHSRN (SEQ ID NO:1) peptide or purified plasma fibronectin fragments containing it, and lacking the α4β1 integrin binding site in the IIICS region, are sufficient to stimulate fibroblast invasion of basement membranes in vitro in the presence of serum or under serum-free conditions, while intact plasma fibronectin fails to stimulate fibroblast invasion. Pure PHSRN (SEQ ID NO:1) peptide has also been shown to stimulate keratinocyte invasion of serum-free SU-ECM. Since, during wound reepithelialization, keratinocytes migrate through the connective tissue of the provisional matrix to “wall off” portions of the wound, as well as through the adjacent stroma, it is not surprising that they are also stimulated to migrate through the matrix of SU-ECM invasion substrates by the PHSRN (SEQ ID NO:1) sequence. This suggests that this peptide, or proteinase-resistant forms of it, may have similar effects on fibroblasts, keratinocytes, and monocytes/macrophages in vivo. Recruitment of fibroblasts or monocytes/macrophages whose paracrine, regulatory effects on a variety of neighboring cells are required for the early stages of wound healing is contemplated as a highly efficient and effective way to stimulate the cascade of regulatory interactions involved in wound healing because these cells will secrete the active factors or cytokines in the correct temporal sequences and spatial locations to ensure their optimal activities. Because it efficiently induces keratinocyte migration through the extracellular matrix in vitro, the PHSRN (SEQ ID NO:1) peptide is also likely to stimulate wound reepithelialization directly. The use of the PHSRN (SEQ ID NO:1) peptide or structurally related molecules according to the present invention is to stimulate the entry of cells such as fibroblasts and monocyte/macrophages into the provisional matrix of a wound, so that the entering cells themselves secrete the factors and cytokines active in inducing or potentiating wound healing. The use of the PHSRN (SEQ ID NO:1) peptide or structurally related molecules is also intended to stimulate wound reepithelialization directly by inducing keratinocyte migration through the extracellular matrix.

### D. Peptide-Dendrimers in Cancer Therapy

1. PHSCN (SEQ ID NO:86) Peptides

A common goal in cancer therapy is to induce tumor-selective cell death, while sparing normal cells and tissues. For example, one approach may involve specifically activating tumor cell death machinery (i.e., for example, apoptotic pathways). Although it is not necessary to understand the mechanism of an invention, it is believed that, in one embodiment, a PHSCN (SEQ ID NO:86) peptide (i.e., for example, Ac-PHSCN-NH₂) specifically interacts with (i.e., for example, by attaching to) an activated tumor cell α5β1 integrin receptor and provides antitumorigenic and antimetastatic effects in preclinical cancer models (i.e., for example, prostate cancer) by reducing tumorigenesis, preventing metastasis, and tumor recurrence. For example, after intravenously injecting a biotinylated PHSCN (SEQ ID NO:86) derivative peptide (i.e., for example Ac-PHSCNGGK(biotin)-NH₂ (SEQ ID NO: 105)) into tumor-bearing mice, standard immunohistochemical examination show that this peptide rapidly and selectively binds to DU 145 prostate cancer cells and their associated blood vessels. It is further believed that this interaction results in the prevention of metastasis, micrometastasis, and tumor recurrence for prolonged periods of time, without adverse consequences. van Golen et al., “Suppression Of Tumor Recurrence And Metastasis By A Combination Of The PHSCN Sequence And The Antiangiogenic Compound Tetrathiomolybdate In Prostate Carcinoma” Neoplasia, 4:373-379 (2002). The PHSCN (SEQ ID NO:86) peptide has nearly completed a Phase I clinical trial, without any severe, treatment-associated, adverse events in the treated patients. This Phase I clinical trial included 23 patients, treated thrice-weekly with intravenous PHSCN peptide at doses ranging from 0.1 to 16.0 mg/kg. Each treatment cycle was defined to be one month (4 weeks) of systemic PHSCN treatment. Among seven of the 23 PHSCN-treated patients there were a total of 11 treatment-emergent serious adverse events. Most of the serious adverse events were hospitalizations due to progressive disease or complications of disease. During this Phase I clinical trial, 9 of the 23 PHSCN-treated patients maintained stable disease for extended periods of time: 4 patients for 2-4 treatment cycles (a total of 2-4 months); and 5 patients for greater than 4 treatment cycles (greater than 4 months). Thus, during this Phase I clinical trial, metastatic disease was prevented from progressing for many months in 38% of the cancer patients receiving systemic therapy at modest doses. Table 2 outlines the patient disposition near the completion of the trial.

TABLE 2
Patient Disposition
N = 23

| | |
|---|---|
| Withdrawn | 2 |
| | 1 |
| Continuing | 2 |
| Best Response | |
| Stable Disease¹ | 9 |
| 2-4 cycles | 4 |
| >4 cycles | 5 |
| Progressive Disease | 1 |
| | 4 |

¹defined as at least 2 cycles of treatment

Other studies have also shown the PHSCN (SEQ ID NO:86) peptide (i.e., for example, Ac-PHSCN-NH₂) to be a potent antitumorigenic and antimetastatic agent for MATLyLu prostate cancer. Livant et al., “The PHSCN Sequence As An Anti-Invasive For Human Prostate Carcinoma Cells And As An Anti-Tumorigenic And Anti-Metastatic Agent For Rat Prostate Cancer” Cancer Research 60:309-320 (2000). These studies suggest that, in one embodiment, PHSCN (SEQ ID NO:86) may block tumor cell invasion. In another embodiment, however, PHSCN (SEQ ID NO:86) peptide may also induce apoptosis. Furthermore, thrice-weekly PHSCN (SEQ ID NO:86) peptide intravenous doses (0.1 to 0.5 mg/kg) prevent cancer progression in metastatic ovarian cancer and metastatic prostate cancer patients in the Phase I clinical trial, for over a 6 to 10 month period.

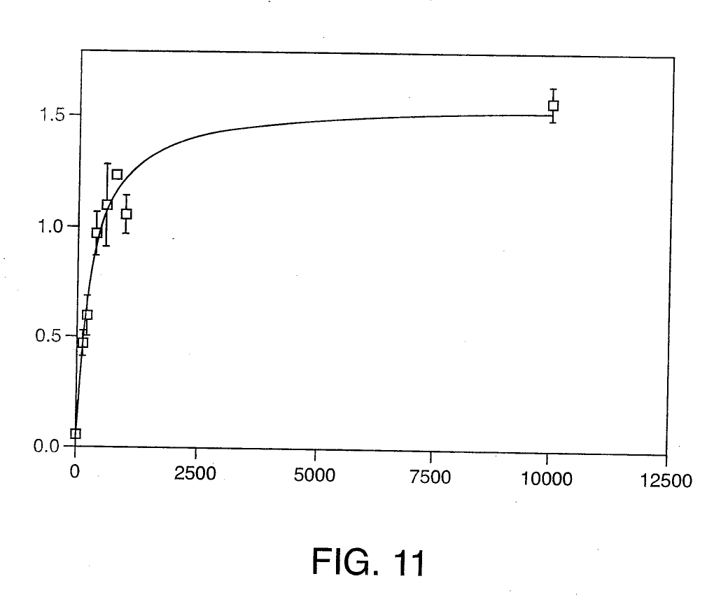

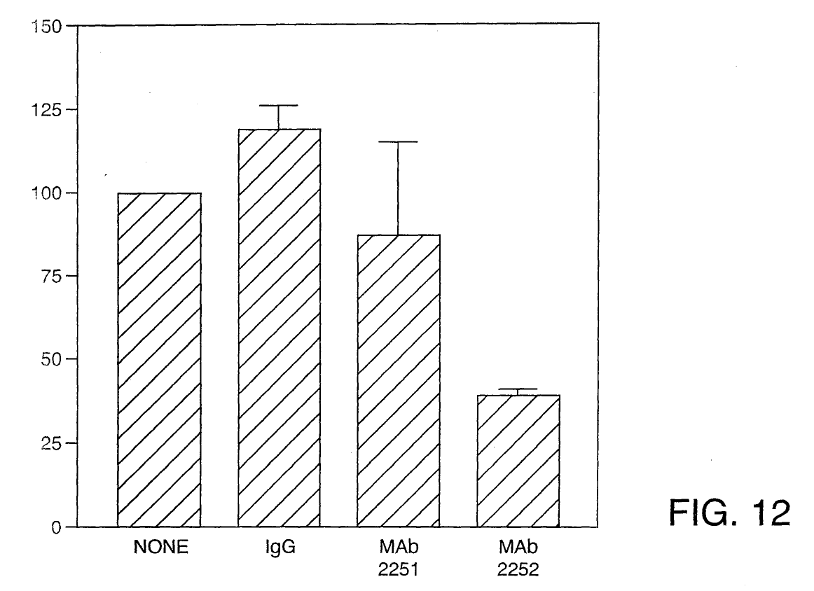

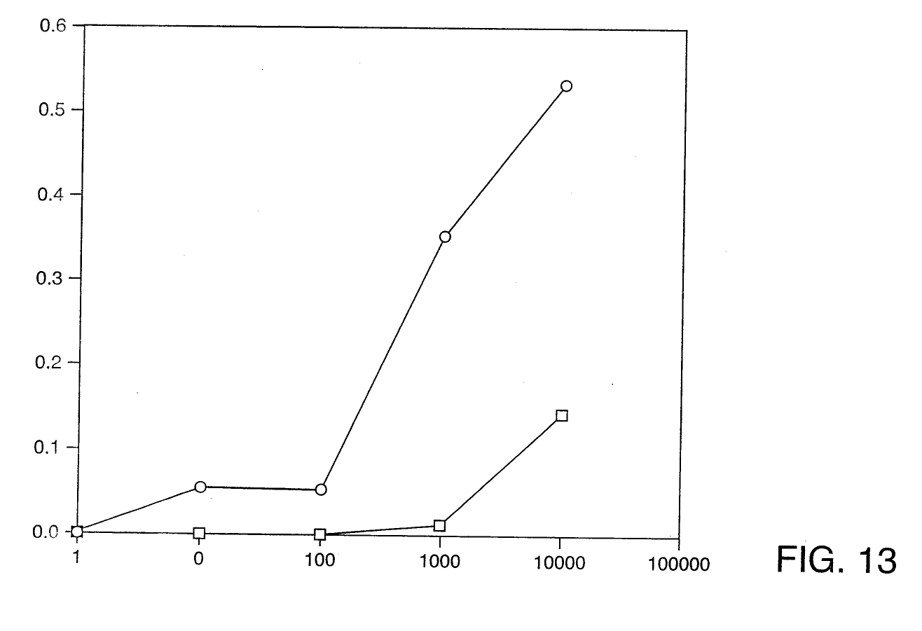

Although it is not necessary to understand the mechanism of an invention, it is believed that PHSCN (SEQ ID NO:86) may interact with the N-terminal regulatory domain of the β₁ integrin subunit to repress the focal adhesion kinase/phosphatidyl-inositol 3-kinase/protein kinase B (FAK/PI3′K/Akt) survival pathway and induce apoptosis (i.e., for example, in cultured DU 145 cells). In one embodiment, a PHSCN (SEQ ID NO:86) peptide binds (i.e., for example, attaches to) immobilized α5β₁ integrin exhibiting a 272 nM K_{d} (See FIG. 11). In another embodiment, PHSCN (SEQ ID NO:86) peptide interacts with (i.e., for example, attaches to) a β₁ subunit N-terminal regulatory domain (See FIG. 12). Although it is not necessary to understand the mechanism of an invention it is believed that PHSCN (SEQ ID NO:86) binds to the β₁ subunit N-terminal regulatory domain because it was observed that PHSCN (SEQ ID NO:86) prebinding reduces MAb 2252 anti-β₁ monoclonal antibody binding (See FIG. 12). Further, it is currently believed that the PHSCN (SEQ ID NO:86) peptide binding epitope comprises amino acids 15-54 and contains 7 cysteine residues. Ni et al., “Integrin Activation By Dithiothreitol Or Me Induces A Ligand—Occupied Conformation And Exposure Of A Novel NH₂—Terminal Regulatory Site On The β1 Integrin Chain” J. Biol. Chem. 273:7981-7987 (1998). It is further believed that the interaction of biotinylated PHSCN (SEQ ID NO:86) peptide with DU 145 cells is greatly increased by the presence of 1 mM MnCl₂ suggesting a preferential binding to activated integrins (See FIG. 13).

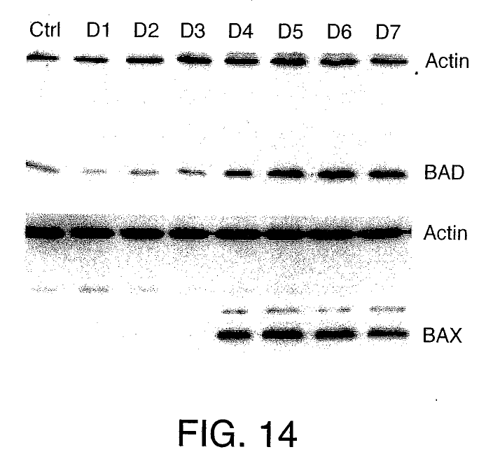

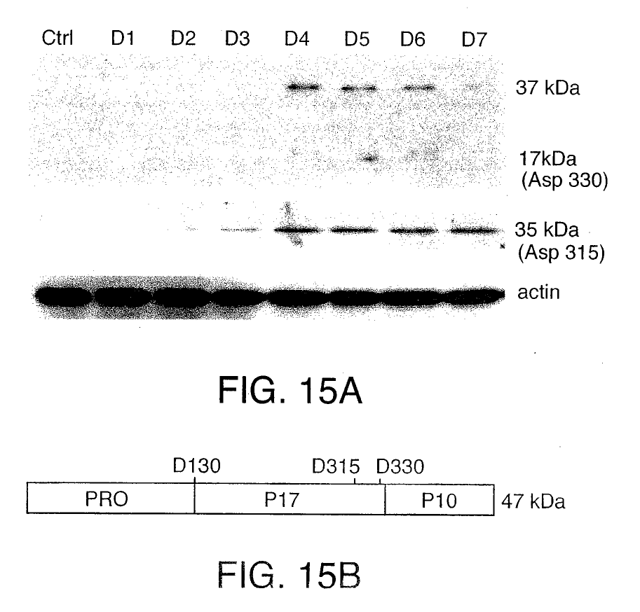

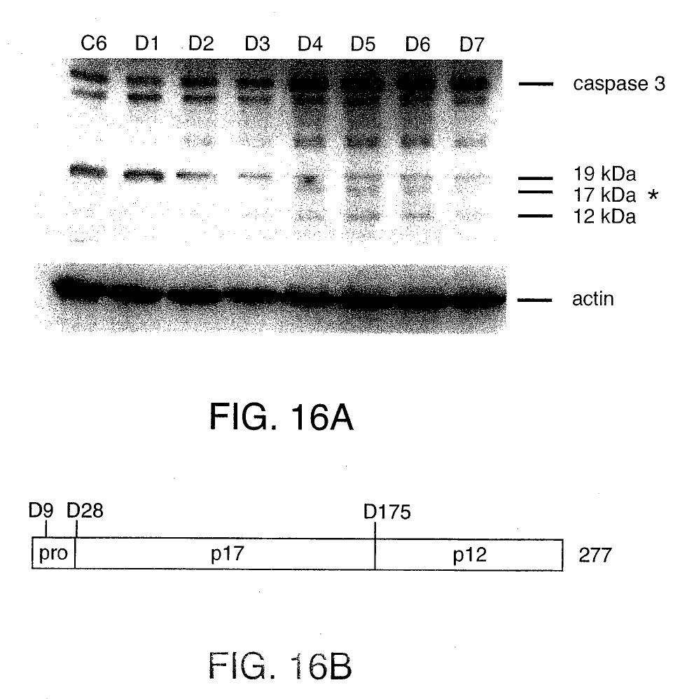

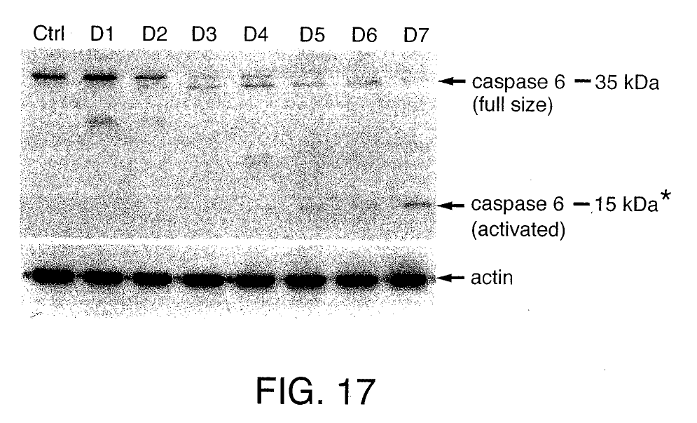

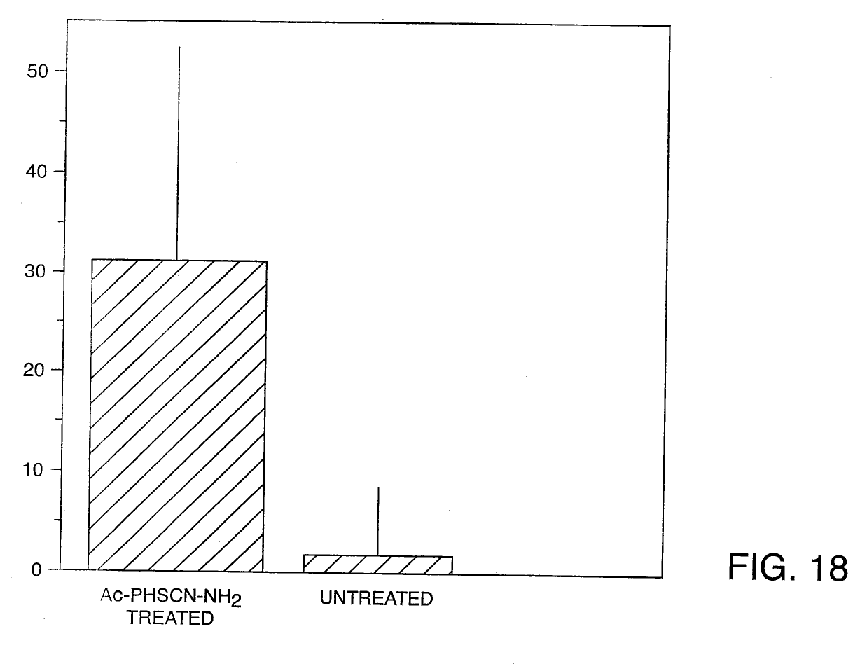

In one embodiment, the present invention contemplates a method to induce apoptosis comprising soluble Ac-PHSCN-NH₂. In one embodiment, Ac-PHSCN-NH₂ induces adherent DU 145 human prostate cancer cell apoptosis. In another embodiment, Ac-PHSCN-NH₂-induced apoptosis comprises upregulation of Bad and Bax protein gene expression. (See FIG. 14). Alternatively, PHSCN (SEQ ID NO:86) peptide may also induce apoptosis by cleaving and activating Caspases 9, 3, and 6 in adherent DU 145 cells (See FIGS. 15, 16, and 17). In one embodiment, therefore, exposure to a soluble PHSCN (SEQ ID NO:86) peptide (i.e., for example, Ac-PHSCN-NH₂) appears to induce apoptosis via intrinsic pathways. PHSCN (SEQ ID NO:86)-induced apoptosis following interaction with α5β₁ integrin was confirmed by the appearance of the cytokeratin 18 epitope visualized in DU 145 cells with an immunoperoxidase-substituted secondary antibody (See FIG. 18). It is known that the cytokeratin 18 epitope results from the caspase-dependent cleavage of the cytoskeleton, and is specific for apoptotic cells. Leers et al, “An Immunohistochemical Study Of The Clearance Of Apoptotic Cellular Fragments” Cell Mol Life Sci. 59:1358-1365 (2002).

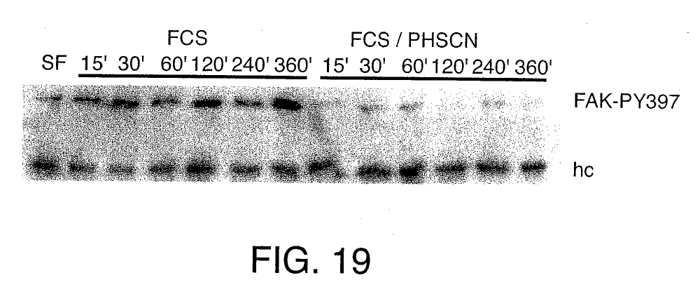

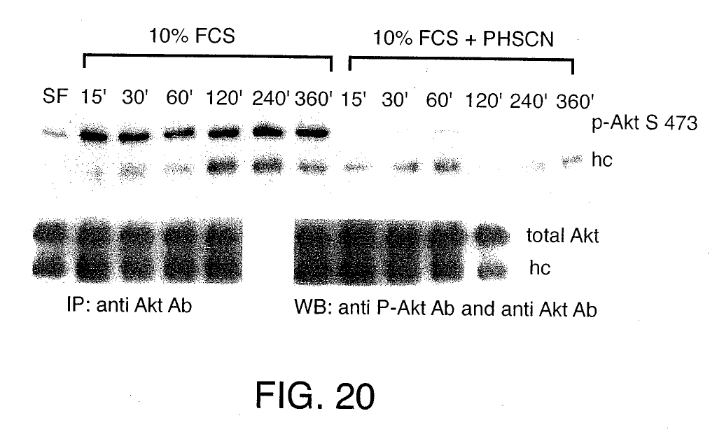

In another embodiment, PHSCN (SEQ ID NO:86) peptide downregulates (i.e., inhibits) serum-induced FAK phosphorylation at tyrosine 397 (Y³⁹⁷). It is believed that Y³⁹⁷ phosphorylation is required for FAK activation. For example, immunoblotting shows that treatment of adherent, serum-starved DU 145 cells overnight with the PHSCN (SEQ ID NO:86) peptide (at a concentration of 1 μg/ml/20,000 cells), in the presence of serum, results in the downregulation of FAK-phosphorylation (See FIG. 19). Similarly, in another embodiment, PHSCN (SEQ ID NO:86) treatment downregulates serum-induced Akt phosphorylation at serine 473 (S⁴⁷³) in adherent DU 145 cells, a site whose phosphorylation is required for Akt activation (See FIG. 20).

Although it is not necessary to understand the mechanism of an invention, it is believed that PHSCN (SEQ ID NO:86) treatment of adherent DU 145 cells also decreases the association of the PI3′-kinase p85 regulatory subunit with FAK (data not shown). It is further believed that the PHSCN (SEQ ID NO:86) peptide appears to interact with α5β₁ integrin that downregulates the FAK/PI3′K/Akt pathway in order to induce cellular apoptosis.

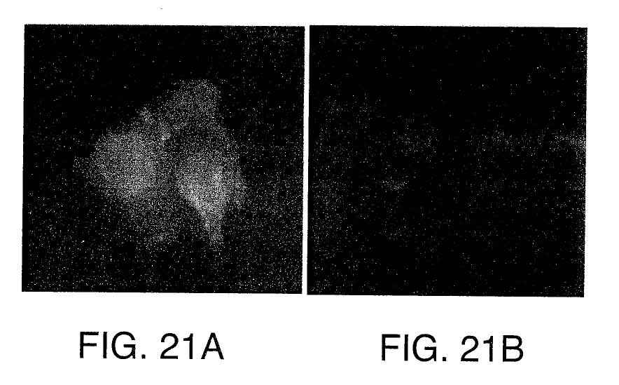

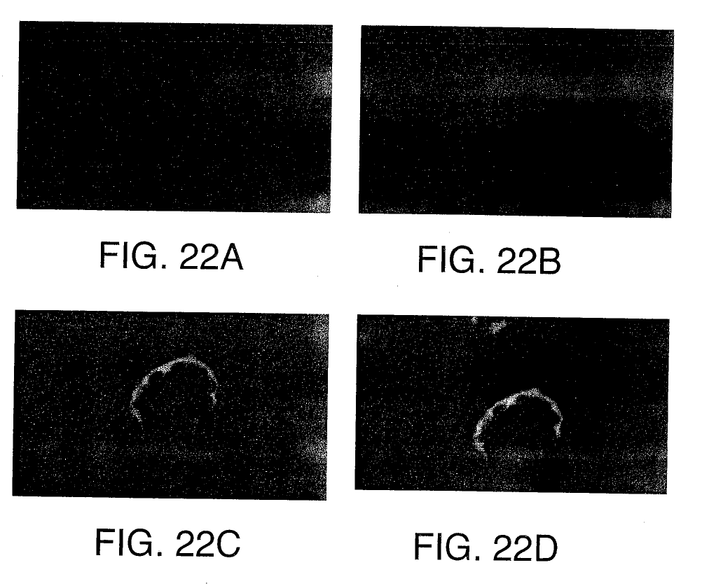

In one embodiment, the present invention contemplates a method comprising providing a PHSCN (SEQ ID NO:86)-peptide as an apoptosis-inducing agent having selectivity for tumor cells and tumor cell-associated blood vessels. In one embodiment, a PHSCN (SEQ ID NO:86) peptide (i.e., for example, a biotinylated PHSCN-related peptide such as Ac-PHSCNGGK(biotin)-NH₂) is injected intravenously into a subject (i.e., for example, a tumor-bearing nude mice). In another embodiment, the biotinylated PHSCN-related peptide rapidly leaves the circulation and accumulates on the tumor cells and associated blood vessel cells but not on the non-tumor (i.e. host) cells (See FIGS. 21 and 22). In one embodiment, the PHSCN-related peptide is selected from the group comprising Ac-PHSCNGGK(biotin)-NH₂ (SEQ ID NO: 105) and Ac-PHSCN-NH₂ peptide.

In one embodiment, the present invention contemplates a method of treating cancer providing a PHSCN (SEQ ID NO:86) peptide wherein the PHSCN (SEQ ID NO:86) peptide specifically interacts with at least one activated tumor cell integrin and/or tumor-associated blood vessel integrin under conditions such that apoptosis and tumor-selective death is induced, thereby preventing tumor cell invasion. The art has not yet, however, identified a method to induce cell death in a maximum number of tumor cells.

The present invention is not limited to inducing tumor-selective death by apoptosis. Although it is not necessary to understand the mechanism of an invention, it is believed that in some embodiments of the present invention tumor-selective death occurs by necrosis or autophagy (See Example 19). Consequently, embodiments described herein contemplate tumor-selective death comprising either apoptotosis, autophagy, or necrosis.

It is known, however, that apoptosis, autophagy, and necrosis are closely related. Recent investigations have demonstrated the need for a precise differentiation of various forms of cell death such as apoptosis, autophagy, and necrosis. In some forms, apoptosis is identified by cellular shrinking, condensation and margination of the chromatin, and ruffling of the plasma membrane (i.e., for example, blebs) with an eventual cellular break up into apoptotic bodies. Autophagy is characterized by the inclusion of bulk portions of cytoplasm and cytoplasmic organelles into autophagic vesicles, surrounded by a single or a double membrane. These membrane-bound vesicles are delivered to the cell's lysozomal system for degradation. It is believed that some malignant cells respond to chemotherapeutic agents by triggering autophagy; thus, the induction of autophagy could be of great therapeutic utility. Gozuacik et al., “Autophagy as a cell death and tumor suppressor mechanism” Oncogene 23:2891-2906 (2004). Necrosis, however, usually refers to the morphological alterations appearing during cell death. Some skilled in the art believe that apoptosis is a pre-mortal process, while necrosis is a post-mortal condition. Van Cruchten et al., “Morphological And Biochemical Aspects Of Apoptosis, Oncosis And Necrosis” Anat Histol Embryol. 31(4):214-23 (2002). The present invention, however, considers that these three processes may be either a pre- or postmortal condition. Specifically, apoptosis and necrosis may be alternative mechanisms that result in cell death. Autophagy, resulting in cell death, may normally function as a tumor suppressor mechanism; hence its therapeutic enhancement could be high desirable.

Apoptosis usually involves physiologic and pathologic stimuli, wherein a full expression triggers a signaling cascade in which caspase activation plays a central role. Knockout mice lacking key genes encoding proteins constituting the core apoptotic cascade are known that point to a possible functional hierarchy of the mechanisms controlling apoptosis. Eliminating genes controlling caspase-dependent apoptosis can convert an apoptotic phenotype to a necrotic one, both in vitro and in vivo. This suggests that necrosis and apoptosis represent morphologic expressions of a shared biochemical network through both caspase-dependent mechanisms as well as non-caspase-dependent effectors such as cathepsin B and apoptosis-inducing factor. Zeiss C. J., “The Apoptosis-Necrosis Continuum Insights From Genetically Altered Mice” Vet Pathol. 40(5):481-95 (2003).

The present invention suggests that differences between apoptosis and necrosis may be based on immunology. For example, there is a substantive immunological difference between Copenhagen rats and the athymic nude mice. The Copenhagen rats have a normal cellular immune system. The athymic nude mice, however, do not have a cellular immune system. This immunological difference may explain some differences in the data describing apoptotic and necrotic embodiments described herein.

When DU145 cells are injected into athymic nude mice, Ac-PHSCN-NH₂ only has an antimetastatic effect (i.e., without an anti-tumorigenesis effect). Microcellular analysis indicates that the antimetastatic effect was a consequence of apoptosis. Cell degradation following apoptosis requires macrophage digestion and does not involve the cellular immune system because the cell membrane does not breakdown.

On the contrary, however, when DU145 cells were injected into Copenhagen rats, Ac-PHSCN-NH₂ was effective in blocking tumorigenesis and had an antimetastatic effect. Microcellular analysis indicates that the antitumorigenic/antimetastatic effect in Copenhagen rats was a consequence of cellular necrosis. Cell degradation following cellular necrosis requires an active cellular immune system response because the cell membrane does breakdown.

Consequently, it is believed that in humans, which have an active cellular immune system, PHSCN-treatment may induce tumor cell death by necrotic breakdown which could activate the cellular immune system against tumor-specific antigens. One having skill in the art would recognize that this result would provide a synergistic immune response specific against the tumor cell. While cell necrosis is a normal end-point of apoptosis, under certain circumstances cell necrosis may be directly induced, thereby bypassing apotosis.

This hypothesis is supported by observations that intracellular macromolecules are released into the blood stream from dying tumor cells. Intracellular macromolecule release into blood would be expected to indicate apoptotic tumor cell death. For example, cytokeratin-18 (CK18) is cleaved by caspases during apoptosis. However, CK18 measurements in patient sera suggest that tumor apoptosis may not necessarily be the dominating death mode in many tumors in vivo. Linder et al., “Determining Tumor Apoptosis And Necrosis In Patient Serum Using Cytokeratin 18 As A Biomarker” Cancer Lett. 214(1):1-9 (2004).

2. Dendrimers

It is known that branched polylysine core dendrimers, as well as the peptide-substituted polylysine dendrimers are stable in solution over a wide pH range. Tam, J. P., “Recent Advances In Multiple Antigen Peptides” J. Immunol. Meth. 196:17-32 (1996). Further, these branched polylysine core dendrimers are known to have a variety of branching levels that provide several alternative numbers of attached peptides. Sadler et al., “Peptide Dendrimers: Applications And Synthesis. Rev. Mol. Biotechnol. 90:195-229 (2002).

Unlike a soluble peptide monomer, a dendrimer allows multiple receptor/ligand interactions to occur in a very small space, thereby producing a ligand cluster. In one embodiment, the multiple receptor/ligand interaction (i.e., for example, that which is produced by a ligand cluster) greatly increases peptide binding affinity that are attached to, for example, a branched polylysine dendrimer. Furthermore, in another embodiment, a ligand cluster improves interaction with cell surface receptors wherein the biological effects of receptor activation are amplified.

The branched, polylysine dendrimer cores utilized in one embodiment of the present invention are not known to induce immune responses. Posnett et al., “A Novel Method For Producing Anti-Peptide Antibodies. Production Of Site-Specific Antibodies To The T Cell Antigen Receptor Beta-Chain” J. Biol. Chem. 263:1710-1725 (1988); and Del Giudice et al., “A Multiple Antigen Peptide From The Repetitive Sequence Of The Plasmodium malariae Circumsporozoite Protein Induces A Specific Antibody Response In Mice Of Various H-2 Haplotypes” Eur. J. Immunol. 20:1619-1622 (1990). Conversely, immune responses are not currently known to affect peptide dendrimer-based therapies. Nomizu et al., “Multimeric Forms Of Tyr-Ile-Gly-Ser-Arg (YIGSR) Peptide Enhance The Inhibition Of Tumor Growth And Metastasis” Cancer Res. 53:3459-3461 (1993). Any potential immunogenicity that might occur during prolonged dendrimer therapies may be avoided by using non-immunogenic materials (i.e., for example, polystyrene, poly(amido amine), or other nonproteinaceous cores). For example, polystyrene dendrimers, as well as poly(amido amine) dendrimers have been utilized to encapsulate chemotherapeutic and other agents. Khopade et al., “Stepwise Self-Assembled Poly(amido amine) Dendrimer And Poly(styrene sulfonate) Microcapsules As Sustained Delivery Vehicles” Biomacromolec. 3:1154-1162 (2002).

A major advantage of dendrimer-mediated drug therapy over conventional monomer drug therapy offers multiple attachment sites thus allowing chemotherapeutic agent targeting specifically to tumor cells that maximizes therapeutic efficacy while minimizing toxic exposure to the non-tumor cells. In one embodiment, the present invention contemplates a dendrimer core to which is attached a ligand and a chemotherapeutic agent, wherein the ligand specifically binds to tumor cells, and the chemotherapeutic agent inhibits tumor cell proliferation. In one embodiment, the dendrimer core provides a targeted delivery of the chemotherapeutic agent to the tumor cells. In one embodiment, the ligand comprises a PHSCN (SEQ ID NO:86) peptide

In one embodiment, the present invention contemplates a method of treating cancer comprising neutron capture therapy. In one embodiment, neutron capture therapy comprises; i) administering a chemotherapeutic agent, wherein the agent contains a chemical isotope having a high affinity for thermal neutrons; ii) exposing a patient to thermal neutrons wherein at least a portion of the thermal neutrons are captured by the chemotherapeutic agent, thereby inducing a localized, biologically-destructive nuclear reaction. In one embodiment, the chemotherapeutic agent comprises methotrexate, wherein the tumor cells are believed to be overexpressing a folate receptor. In another embodiment, the chemotherapeutic agent comprises boron. In another embodiment, the is chemotherapeutic agent comprises gemcitabine, 5-fluoruracil, cisplatin, estramustine, doxorubicin, paclitaxel, or other agents that function to block cancer cell proliferation by inhibiting various aspects of DNA synthesis, DNA strand separation, or the segregation of the daughter chromosomes to opposite regions of the dividing cell. Hence, the PHSCN ligand would have the effect of localizing the dendrimer-coupled chemotherapeutic agent specifically to tumor cells and their associated blood vessels, thus maximizing its therapeutic efficacy while it minimizing harmful effects to non-tumor cells. In another embodiment, the chemotherapeutic agent comprises an inhibitor of a matrix metalloproteinase. Here, the PHSCN ligand would have the effect of localizing the dendrimer-coupled MMP inhibitor to tumor cells and their associated blood vessels to maximize its invasion-inhibitory potency. In one embodiment, the neutron capture therapy comprises antibody- or receptor-substituted poly(amido amine) dendrimers, wherein the tumor cells are believed to be overexpressing endothelial growth factor receptor. In another embodiment, the dendrimers comprise concentric shells of branching β-alanine. Quintana et al., “Design And Function Of A Dendrimer-Based Therapeutic Nanodevice Targeted To Tumor Cells Through The Folate Receptor” Pharm. Res. 19:1310-1316 (2002); Wu et al., “Site-Specific Conjugation Of Boron-Containing Dendrimers To Anti-EGF Receptor Monoclonal Antibody Cetuximab (IMC-C225) And Its Evaluation As A Potential Delivery Agent For Neutron Capture Therapy” Bioconjug. Chem. 15:185-194 (2004). Kojima et al., “Synthesis Of Polyamidoamine Dendrimers Having Poly(ethylene glycol) Grafts And Their Ability To Encapsulate Anticancer Drugs” Bioconjugate Chem. 11:910-917 (2000); and Tomalia et al., “Starburst Dendrimers: Molecular-Level Control Of Size, Shape, Surface Chemistry, Topology, And Flexibility From Atoms To Macroscopic Matter” Angew. Chem. Int. Ed. 29: 138-175 (1990).

One advantage of the present invention is that, in one embodiment, complex traditional synthetic chemistry protocols are avoided for the attachment of peptides or proteins to dendrimers that create unwanted side reactions. In one embodiment, the present invention contemplates standard peptide synthesis procedures (i.e., for example, Fmoc protocols) which attach pre-formed PHSCN-related peptides to commercially available branching polylysine dendrimer cores (i.e., for example, Nova Biochem/EMD Biosciences, San Diego Calif.; or VivaGel®, Starpharma, Melbourne, Australia). Furthermore, the CORE facility at the University of Michigan has the capacity to custom design and synthesize dendrimers. Although it is not necessary to understand the mechanism of an invention, it is believed that these Fmoc reactions are very complete and side reactions are minimized.

In one embodiment, the present invention contemplates a composition comprising a dendrimer, wherein approximately two (2)-thirty (30), preferably four (4)-twenty (20), but more preferably eight (8)-sixteen (16) PHSCN (SEQ ID NO:86) peptide derivatives are attached, thereby creating PHSCN-substituted dendrimers. In one embodiment, these dendrimers comprising PHSCN (SEQ ID NO:86) peptide derivatives induce apoptosis in human prostate cancer cells, both in vitro and in vivo at various potencies. In another embodiment, these dendrimers comprising PHSCN (SEQ ID NO:86) inhibit invasion in human prostate cancer cells, both in vitro and in vivo. In another embodiment, the PHSCN (SEQ ID NO:86) dendrimers further comprise substituted chemotherapeutic agents to create a PHSCN (SEQ ID NO:86)-dendrimer chemotherapeutic complex. Although it is not necessary to understand the mechanism of an invention, it is believed that a PHSCN (SEQ ID NO:86)-dendrimer chemotherapeutic complex may be delivered to tumor cells to provide a precisely targeted, combination therapy for cancer (i.e., for example, prostate cancer).

In another embodiment, a peptide dendrimer comprises a scrambled control peptide. In one embodiment, the scrambled control peptide comprises an HSPNC amino acid sequence (SEQ ID NO:107). In another embodiment, a scrambled control peptide is tested for antitumorigenic and antimetastatic activity. Although it is not necessary to understand the mechanism of an invention, it is believed that HSPNC peptide-dendrimers are devoid of antitumorigenic and antimetastatic activity because they do not interact with α5β1 integrin molecule.

### E. Pancreatic Cancer

Pancreatic cancer kills 28,000 Americans annually and is the fourth leading cause of cancer death in the United States. Niederhuber et al., “The National Cancer Data Base Report On Pancreatic Cancer” Cancer 76:1671-1677 (1995); Washaw et al., “Pancreatic Carcinoma” N. Engl. J. Med. 326: 455-465 (1992). In general, cancer prognosis is known to depend on the tumor stage at the time of diagnosis. Pancreatic cancer, however, metastasizes very early into local tissues, as well as lymphatic, venous, peritoneal, and perineural sites. This creates an unfortunate situation where even early diagnosis can, and does, lead to a poor prognosis. For example, clinical studies have shown that regional lymph node metastases are found in 30% of patients with very small primary pancreatic tumors, and 64% of patients with a T1 primary cancer have lymph node involvement. Hermanek et al., “Early-Stage Pancreatic Ductal Adenocarcinoma Surgery” Int. J. Pancreatol. 16:302-304 (1994). Additionally, other studies demonstrate that pancreatic cancer metastases involve: i) lymphatic vessels (89%); ii) lymph nodes (77%); iii) intrapancreatic neural invasion (92%); and iv) extremely painful extrapancreatic nerve plexus invasion (45%). Nagakawa et al., “A Clinicopathologic Study On Neural Invasion In Cancer Of The Pancreatic Head” Cancer 69:930-935 (1992); and Cubilla et al., “Morphological Lesions Associated With Human Primary Invasive Nonendocrine Pancreas Cancer” Cancer Res 36:2690-2698 (1976). In autopsy studies, pancreatic cancer dissemination was observed in: i) peritoneal cavity (40%); ii) “Morphological Lesions Associated With Human Primary Invasive Nonendocrine Pancreas Cancer” Cancer Res 36:2690-2698 (1976); Watanapa et al., “Surgical Palliation For Pancreatic Cancer: Developments During The Past Two Decades” Br. J. Surg. 79:8-20 (1992); Lee et al., “Carcinoma Of The Pancreas And Periampullary Structures. Pattern Of Metastases At Autopsy” Arch. Pathol. Lab. Med. 108:584-587 (1984). It is clear that even when a pancreatic primary tumor is small enough to surgically remove completely and metastases are not grossly evident, early micrometastasis limits the possibility of cure. Recent studies show that pancreatic cancer patients having lymph node metastases and/or blood vessel invasion have a poor prognosis, even if the primary tumor is completely removed. Cameron et al., “Factors Influencing Survival After Pancreaticoduodenectomy For Pancreatic Cancer” Am. J. Surg. 161:120-125 (1991); Ishikawa et al., (1988) Practical Usefulness Of Lymphatic And Connective Tissue Clearance For The Carcinoma Of The Pancreas Head” Ann Surg. 208:215-220 (1988); Geer et al., “Prognostic Indicators For Survival After Resection Of Pancreatic Adenocarcinoma” Am. J. Surg. 165:68-73 (1993). In fact, an adenocarcinoma in the head of the pancreas (which normally has a better prognosis than pancreatic tail adenocarcinomas) only has a 28% survival rate following pancreatectomy (61 patients). Trede, M., “The Surgical Options” In: Surgery Of the Pancreas, Trede, M., Carter, D. C., Edinburgh: Churchill Livingstone (1993).

Thus, there is an urgent need of a well tolerated, long term, systemic therapy to prevent metastasis progression in pancreatic cancer. In one embodiment, the present invention contemplates a method comprising a PHSCN-substituted dendrimer to treat pancreatic cancer metastasis progression. The Ac-PHSCN-NH₂ monomer peptides are effective in treating prostate cancer. For example, 80 nude mice were given thrice-weekly doses of Ac-PHSCN-NH₂ peptides (50 mg/kg) after surgical excision of large, untreated DU 145 human prostate carcinoma primary tumors. The Ac-PHSCN-NH₂ monomer peptide administration prevented metastasis progression in 100% of the treated mice (40), while all the untreated mice (40) rapidly succumbed to metastasis. Eight months later, the treated mice showed no obvious loss of vigor or difficulty in healing. van Golen et al., “Suppression Of Tumor Recurrence And Metastasis By A Combination Of The PHSCN Sequence And The Antiangiogenic Compound Tetrathiomolybdate In Prostate Carcinoma” Neoplasia 4(5):373-379 (2002). Further, potent antitumorigenic and antimetastatic effects with systemic PHSCN therapy to treat MATLyLu rat prostate cancer has also been demonstrated. Livant et al., “The PHSCN Sequence As An Anti-Invasive For Human Prostate Carcinoma Cells, And As An Anti-Tumorigenic And Anti-Metastatic Agent For Rat Prostate Cancer” Cancer Research 60:309-320 (2000). In one embodiment, a peptide-substituted dendrimer comprising PHSCN is administered to a human, wherein said dendrimer is well tolerated.

Although it is not necessary to understand the mechanism of an invention, it is believed that, unlike a soluble peptide monomer, a peptide-substituted dendrimer allows multiple receptor/ligand interactions to occur in a very small space (i.e., for example, a ligand cluster). Further, it is believed that this multiplicity of interaction leads to the greatly increased binding affinity of peptides attached to branched, polylysine dendrimers, as well as to the clustering of many interacting receptors on the cell surface to amplify the cellular effects of the receptor interaction. Sadler et al., “Peptide Dendrimers: Applications And Synthesis” Rev. Mol. Biotechnol. 90:195-229 (2002).

One embodiment of the present invention is related to compositions and methods comprising a malignant cancer having an α5β1 integrin (i.e., for example, pancreatic cancer). In one embodiment, the present invention contemplates a method of treating a malignant cancer wherein α5β1 integrin is a therapeutic target. In one embodiment, a PHSCN peptide (i.e., for example, Ac-PHSCN-NH₂ or a PHSCN-comprising dendrimer) binds to malignant cancer α5β1 integrins, thereby blocking invasion and inducing tumor cell death by repressing the FAK/PI3′K/Akt pathway. Although it is not necessary to understand the mechanism of an invention, it is believed that PHSCN selectively binds pancreatic tumor cells and associated blood vessels, as shown by intravenous injection of biotinylated-PHSCN into tumor-bearing mice and immunohistochemical examination of excised tumors. As a promising therapeutic agent, it is further believed that PHSCN, PHSCN-substituted dendrimer and derivatives thereof, have increased potency over standard chemotherapeutic regimens that may effectively target the most aggressive cancers.

One example of an aggressive cancer is pancreatic cancer. Because of pancreatic cancer's propensity to metastasize, ductal pancreatic adenocarcinoma has a 20% two-year survival rate. Real F.X., “A ‘Catastrophic Hypothesis’ For Pancreas Cancer Progression” Gastroenterol. 124:1958-1964 (2003). To develop an effective pancreatic cancer treatment, one embodiment of the present invention contemplates synthesizing PHSCN-substituted dendrimers, evaluating PHSCN-substituted dendrimers regarding antitumorigenic and antimetastatic activities, and administering PHSCN-substituted dendrimers to human pancreatic cancer cells to induce apoptosis and inhibit cellular invasion, both in vitro and in vivo. Specifically, one embodiment comprises PHSCN peptides, attached to branched, polylysine dendrimers of different sizes, that block invasion and reduce survival in human pancreatic cancer cells.

Many metastatic human pancreatic cancer cell lines, including BxPC-3, AsPC-1, CAPAN-1, and CAPAN-2 are known to express α5β1 integrin, but not α4β1 integrin fibronectin receptors. Lohr et al., “Expression And Function Of Receptors For Extracellular Matrix Proteins In Human Ductal Adenocarcinomas Of The Pancreas” Pancreas 12:248-259 (1996). Although it is not necessary to understand the mechanism of an invention, it is believed that since the invasive nature of α5β1-positive, α4β1-negative breast and prostate cancer cells are plasma fibronectin-dependent, it is likely that invasive pancreatic cancer cells are also plasma fibronectin-dependent. Livant et al., “The PHSCN Sequence As An Anti-Invasive For Human Prostate Carcinoma Cells, And As An Anti-Tumorigenic And Anti-Metastatic Agent For Rat Prostate Cancer” Cancer Research 60:309-320 (2000); Jia et al., “Integrin Fibronectin Receptors In MMP-1 Dependent Invasion By Breast Cancer And Mammary Epithelial Cells” Cancer Research, in press (2004); Ignatoski et al., “p38 MAPK Induces Cell Surface α4 Integrin Down-Regulation to Facilitate erbB-2 Mediated Invasion” Neoplasia 5(2): 128-134 (2003); and Roklin et al., “Expression Of Cellular Adhesion Molecules On Human Prostate Tumor Lines” Prostate 26:205-212 (1995). Consequently, in one embodiment, systemic PHSCN peptide (i.e., Ac-PHSCN-NH₂ or a PHSCN-substituted dendrimer) administration to a patient reduces tumorigenesis, inhibits metastasis, and induces apoptosis in pancreatic cancer tumors. In one embodiment, PHSCN-dendrimers are administered, wherein the dendrimers have an increased efficacy as compared to the monomeric Ac-PHSCN-NH₂ peptide.

In one embodiment, the present invention contemplates a method for synthesizing dendrimers comprising PHSCN, comprising; i) providing commercially obtained branched polylysine dendrimer cores (i.e., for example, having 4, 8, or 16 sites for peptide attachment); ii) covalently attaching the core to an inert polystyrene polymer; and iii) attaching a protein selected from the group comprising PHSCN, PHSCNGG, PHSCNGGK, or HSPNC peptides on the dendrimer cores using solid phase peptide synthesis and employing current methods in F-moc chemistry. Ambulos, N. “Analysis Of Synthetic Peptides” In: Solid Phase Synthesis, Kates, S. A., Albericio, F., eds. Marcell Dekker, Inc., New York., pp. 782-805 (2000). After synthesis, the peptides may be analyzed by HPLC-MS (high pressure liquid chromatography-mass spectrometry) and the desired product isolated by HPLC. Additional analysis using amino acid sequencing may be performed (i.e., for example, Edman degradation) to verify the peptide-dendrimer composition.

In sum, the present invention contemplates a composition comprising at least one PHSCN peptide attached to a branched dendrimer. Moreover, the present invention contemplates methods of treating proliferative diseases, comprising administering to a patient in need of such treatment an effective amount of at least one PHSCN peptide attached to a branched dendrimer. Alternatively, the present invention contemplates methods of preventing tumor metastasis or inhibiting proliferation of a cell comprising administering to a patient in need of such treatment an effective amount of at least one PHSCN peptide attached to a branched dendrimer.

### EXPERIMENTAL

The following examples serve to illustrate certain preferred embodiments and aspects of the present invention and are not to be construed as limiting the scope thereof.

In the experimental disclosure which follows, the following abbreviations apply: eq (equivalents); M (Molar); μM (micromolar); mM (millimolar); N (Normal); mol (moles); mmol (millimoles); μmol (micromoles); nmol (nanomoles); g (grams); mg (milligrams); μg (micrograms); L (liters); ml (milliliters); μl (microliters); cm (centimeters); mm (millimeters); μm (micrometers); nm (nanometers); ° C. (degrees Centigrade); mAb (monoclonal antibody); MW (molecular weight); PBS (phophate buffered saline); U (units); d (days).

### EXAMPLE 1

### Production of Fibronectin-Free Substrates

This example describes a purification approach for removal of plasma fibronectin (and/or cellular fibronectin) from a substrate (Matrigel). In this example, removal was attempted by affinity chromatography over Gelatin-Sepharose (a technique which can be used to remove plasma fibronectin from fetal calf serum).

The Gelatin-Sepharose beads were obtained from Pharmacia (Catalog# 17-0956-01). Two Kontes columns were set up with about 2 mls of Gelatin-Sepharose beads at 4° C. to prevent gelling of the Matrigel. The columns were then rinsed with about 10 column volumes of PBS to remove the preservative from the beads. The columns were drained to the top of the beads; then Matrigel was carefully added to the column. Once the Matrigel had entered the column, PBS was added to the top of the column. The Matrigel which was passed over the first column was collected and passed over the second column. The fibronectin-depleted Matrigel collected from the second column was plated on 48-well plates (150 μl/well), sterilized under a UV light for 10 minutes and incubated at 37° C. overnight. The Matrigel treated in this manner failed to form a gel at 37° C.

### EXAMPLE 2

### Production of Fibronectin-Free Substrates

This example describes a purification approach for removal of plasma fibronectin (and/or cellular fibronectin) from a substrate (Matrigel). In this example, removal was attempted by successive panning on gelatin. Eight wells of 24-well plate were coated with a 2% gelatin solution (the gelatin was obtained from Becton Dickinson Labware, Catalog #11868). The wells were filled with the gelatin solution which had been heated to 50° C. and incubated for 3 minutes. Then the solution was removed and the wells were allowed to air dry. Following drying, the wells were thoroughly rinsed with ddH₂O followed by two rinses with PBS. The plates were again allowed to dry; thereafter they were stored at −20° C. until use. Matrigel was thawed on ice and then added to one of the wells of a gelatin-coated plate (between 800 μl and 1 ml of Matrigel was added to a well of a 24-well plate). The plate was placed in a bucket of ice in a 4′I C room on an orbital shaker where the Matrigel was incubated in the well for two hours (although overnight incubation can be used). Following the incubation, the Matrigel was moved from the first well to a second well and then incubated for two hours under the same conditions. This process was repeated until the Matrigel had been incubated on all eight wells of the gelatin-coated plate.

Following the depletion of the Matrigel, it was collected in Eppendorf tubes. It was then plated on a 48-well plate 150 μl/well), sterilized under a UV light for 10 minutes and incubated at 37° C. overnight. The Matrigel formed as gel and the following day, cells were added to each well.

### EXAMPLE 3

### Production of Fibronectin-Free Substrates

This example describes a purification approach for removal of plasma fibronectin (and/or cellular fibronectin) from a substrate (Matrigel). In this example, removal was attempted by gelatin panning followed by antibody panning.

Anti-fibronectin antibody-coated wells: Wells of a 24-well plate were coated with an anti-fibronectin antibody. A mouse monoclonal antibody to human fibronectin was obtained from Oncogene Science (Catalog #CP13). Each well was incubated with 1 ml of antibody at a concentration of 30 μl/ml for 2 hours at room temperature. Each well was then incubated with a solution of 3% BSA in PBS for 2 hours at room temperature. Following the two incubation periods, the wells were thoroughly washed with PBS and stored at −20° C. until use.

Depleting Matrigel of Fibronectin: Matrigel was panned over eight gelatin-coated wells (as described above in Example 2) to remove most of the fibronectin and its fragments. Thereafter, the Matrigel was placed in the antibody-coated wells to remove any remaining fragments of fibronectin which contain the cell-binding domain but not the gelatin-binding domain. The Matrigel was incubated in an ice bucket on an orbital shaker at 4° C. for 2 hours. Once the Matrigel was depleted, it was collected in Eppendorf tubes. The firbonectin-depleted Matrigel was plated on a 48-well plate (150 μl/well), sterilized under a UV light for 10 minutes and incubated at 37° C. overnight. The Matrigel formed a gel and the following day, cells were added to the wells.

### EXAMPLE 4

### Inducing Invasive Behavior of Tumor Cells

In this example, the role of plasma fibronectin in inducing the invasive behaviors of metastatic breast and prostate cancer cells is demonstrated. Human breast carcinoma cell lines SUM 52 PE and SUM 44 PE were originally cultured from the pleural effusions of patients with metastatic breast cancer; and SUM 102 was cultured from a primary, microinvasive breast carcinoma. Ethier et al., “Differential Isolation Of Normal Luminal Mammary Epithelial Cells And Breast Cancer Cells From Primary And Metastatic Sites Using Selective Media” Cancer Research 53: 627-635 (1993). The DU 145 metastatic human prostate cancer cell line was originally cultured from a brain metastasis. Stone et al., “Isolation Of A Human Prostate Carcinoma Cell Line (DU 145)” Int. J. Cancer 21: 274-281 (1978). These cells express α3β1 which has been shown to repress metalloproteinase transcription upon binding the connecting segment of plasma Fn. These cell lines can all be cultured under serum-free conditions; thus they are ideal for use in serum-free invasion assays on SU-ECM.

Adult Strongylocentrotus purpuratus sea urchins were obtained from Pacific BioMarine, and their embryos were cultured to the early pluteus stage in artificial sea water at 15° C. SU-ECM were prepared from them by treatment with nonionic detergent and strerilized by dilution in the appropriate media.

Cells were harvested by rinsing in Hanks' balanced salt solution, followed by brief treatment with 0.25% trypsin, 0.02% EDTA, and pelleting and resuspension in the appropriate medium with or without 5% FCS at a density of about 50,000 cells per ml. When appropriate, purified bovine plasma fibronectin (Sigma), purified 120 kDa chymotryptic fragment (Gibco BRL), PHSRN (SEQ ID NO:1) or PHSCN (SEQ ID NO:86) peptides (synthesized at the Biomedical Research Core Facilities of the University of Michigan), or GRGDSP (SEQ ID NO:83) or GRGESP (SEQ ID NO:84) peptides (Gibco BRL) were added to the resuspended cells prior to placement of the cells on SU-ECM. In each well of a plate used for an invasion assay, SU-ECM were placed in 0.5 ml of the appropriate medium, and 0.5 ml of the resuspended cells dropped on their exterior surfaces. Invasion assays were incubated 1 to 16 hours prior to assay. If some circumstances, invasion assays were fixed in phosphate-buffered saline (PBS) with 2% formaldehyde for 5 minutes at room temperature, then rinsed into PBS.

Invasion assays were coded and scored blindly by microscopic examination under phase contrast at 200- and 400-fold magnification. Each cell contacting an SU-ECM was scored for its position relative to the exterior or interior surfaces. A cell was judged to have invaded if it was located on an interior surface below the focal plane passing through the upper surface of the SU-ECM, but above the focal plane passing through its lower surface. The minimum viability of the cells in each assay was always ascertained at the time of assay by determining the fraction of spread, adherent cells on the bottom of each well scored.

An invasion frequency is defined as the fraction of cells in contact with basement membranes which were located in their interiors at the time of assay. Thus, an invasion frequency of 1 denotes invasion by 100% of the cells in contact with basement membranes. Invasion frequencies were determined multiple times for each cell type assayed. For each type of cell assayed the mean and standard deviation of the invasion frequencies were calculated.

The invasion-inducing sequences of plasma fibronectin were mapped to a peptide sequence 5 amino acids long, the PHSRN (SEQ ID NO:1) peptide, for both metastatic breast and prostate cancer cells. Since the PHSRN (SEQ ID NO:1) sequence is present in plasma fibronectin, a significant component of serum, eliciting the regulatory role of this sequence was only possible because of the availability of a serum-free in vitro invasion substrate. It should be noted that neonatal, human fibroblasts are also induced with the PHSRN (SEQ ID NO:1) peptide to invade serum-free SU-ECM. Although fibroblasts do not invade SU-ECM in the presence of serum, the 120 kDa fragment of plasma fibronectin containing the PHSRN (SEQ ID NO:1) sequence can induce fibroblast invasion equally well in the presence of serum or in its absence.

When taken together, the results of experiments showing that the PHSRN (SEQ ID NO:1) sequence of plasma fibronectin induces the invasive behaviors of both metastatic breast and prostate cancer cells, as well as that of normal fibroblasts suggest the intriguing possibility that the invasive behavior associated with tumor cell metastasis may result from defects in the regulation of the normal invasive behaviors associated with wound healing.

### EXAMPLE 5

Testing Tumor Cells On Fibronectin-Depleted Substrates

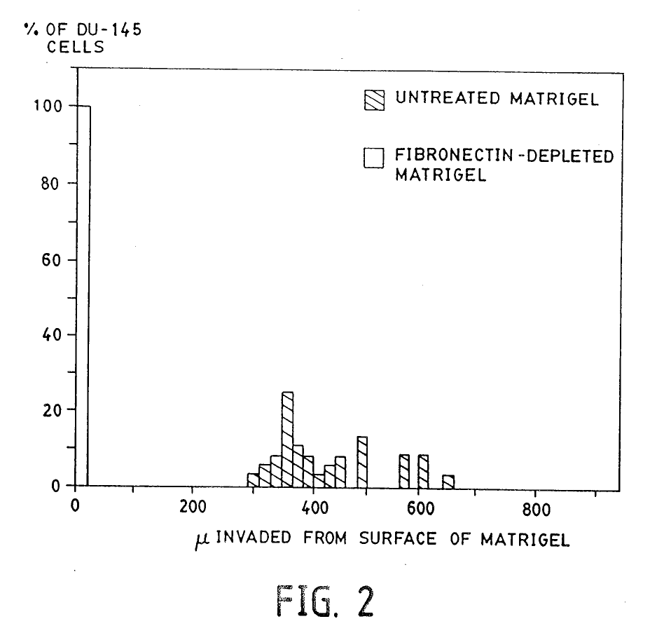

This example describes an approach to test cancer cells in vitro on substrates with and without invasion-inducing agents. The depleted preparation of Matrigel (see Example 2, above) and untreated Matrigel were used to test DU-145 metastatic prostate cancer cells. When plated on the depleted medium, the cancer cells failed to invade the matrix (see FIG. 2). Indeed, it was evident that these cells were sitting on the surface of the depleted Matrigel because the Matrigel surface was slightly tilted; this was visible through the microscope as a gradual progressive, uniform change in the focal plane for the monolayer of DU-145 cells.

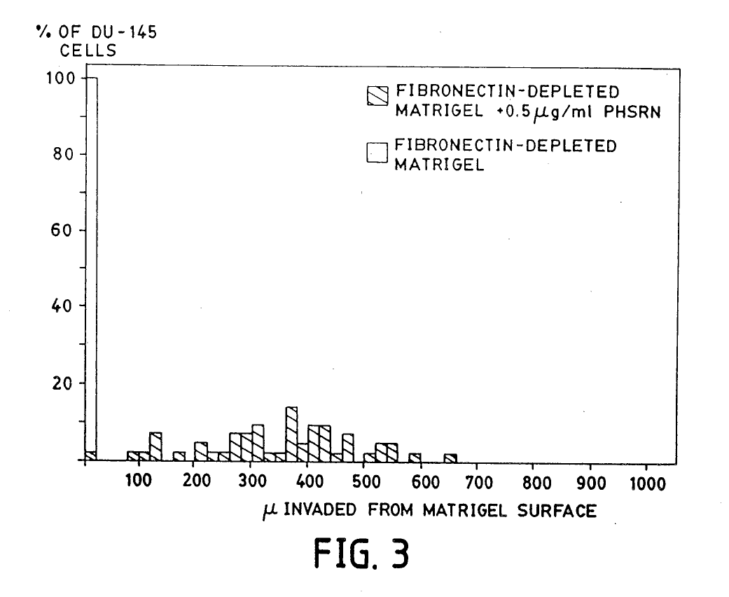

The addition of 0.5 μl/ml of the PHSRN (SEQ ID NO:1) peptide to the depleted Matrigel was sufficient to restore the full DU-145 invasiveness (see FIG. 3). Clearly, gelatin panning removes fibronectin such that cancer cells are unable to invade. Since the addition of PHSRN (SEQ ID NO:1) peptide in solution fully restores the DU-145 invasive phenotype, blocking the effect of PHSRN (SEQ ID NO:1) is an effective strategy for therapeutic intervention in tumor cell invasion and metastasis.

### EXAMPLE 6

### Improving Gelatin Depletion as Measured by Fibroblast Invasiveness

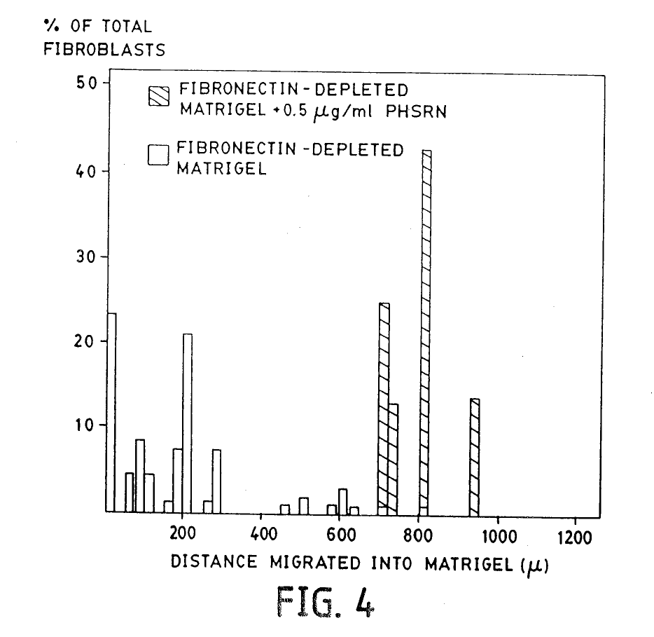

In this example, normal, neonatal fibroblasts were tested on the depleted Matrigel material prepared according to Example 3 above (i.e., antibody depletion). As shown in FIG. 4, palming with an antibody after gelatin depletion improved the method for removal, as measured by the reduced invasiveness of fibroblasts. On the other hand, invasiveness of the fibroblasts could be induced by the addition of the PHSRN (SEQ ID NO:1) peptide.

The success of antibody panning suggests the feasibility of removing other components by the antibody panning methods. Other serum components, such as thrombospondin, growth factors and cytokines are contemplated by the present invention for removal by the appropriate (commercially available) antibody.

### EXAMPLE 7

### Conjugation Of PHSRN-Containing (SEQ ID NO:1) Peptides

In this example, the preparation of a peptide conjugate is described. The synthetic peptide NH₂-PHSRNC (SEQ ID NO:82) can be prepared commercially (e.g., Multiple Peptide Systems, San Diego, Calif.). The cysteine is added to facilitate conjugation to other proteins.

In order to prepare a protein for conjugation (e.g., BSA), it is dissolved in buffer (e.g., 0.01 M NaPO₄, pH 7.0) to a final concentration of approximately 20 mg/ml. At the same time n-maleimidobenzoyl-N-hydroxysuccinimide ester (“MBS” available from Pierce) is dissolved in N,N-dimethyl formamide to a concentration of 5 mg/ml. The MBS solution, 0.51 ml, is added to 3.25 ml of the protein solution and incubated for 30 minutes at room temperature with stirring every 5 minutes. The MBS-activated protein is then purified by chromatography on a Bio-Gel P-10 column (Bio-Rad; 40 ml bed volume) equilibrated with 50 mM NaPO₄, pH 7.0 buffer. Peak fractions are pooled (6.0 ml).

The above-described cysteine-modified peptide (20 mg) is added to the activated protein mixture, stirred until the peptide is dissolved and incubated 3 hours at room temperature. Within 20 minutes, the reaction mixture becomes cloudy and precipitates form. After 3 hours, the reaction mixture is centrifuged at 10,000×g for 10 min and the supernatant analyzed for protein content. The conjugate precipitate is washed three times with PBS and stored at 4° C.

From the above, it should be clear that the present invention provides a method of testing a wide variety of tumor types, and in particular identifying invasive tumors. With a means of identifying such tumors (now provided by the present invention) and distinguishing such tumors from non-invasive cancer, the physician is able to change and/or optimize therapy. Importantly, the antagonists of the present invention (and other drugs developed by use of the screening assay of the present invention) will provide treatment directed an invasive cells (and therefore associated with minimal host toxicity).

### EXAMPLE 8

### Inhibiting Invasion of Human Breast Cancer Cells

In this example, the role of the PHSCN (SEQ ID NO:86) peptide in inhibiting the invasive behavior of metastatic breast cancer cells is demonstrated. The method of Example 4 is employed, with the addition of varying concentrations of the PHSCN (SEQ ID NO:86) peptide.

Example 4 indicates that SUM-52 cells (in medium with 5% fecal calf serum) are induced to invade the SU-ECM substrate in the presence of serum fibronectin or just the PHSRN (SEQ ID NO:1) sequence of fibronectin. Thus, the procedure in Example 4 provides a method for determining the inhibitory potential of the PHSCN (SEQ ID NO:86) peptide by comparing the number of cell invasions in the presence of the PHSCN (SEQ ID NO:86) peptide, with the number of cell invasions in the absence of the PHSCN (SEQ ID NO:86) peptide.

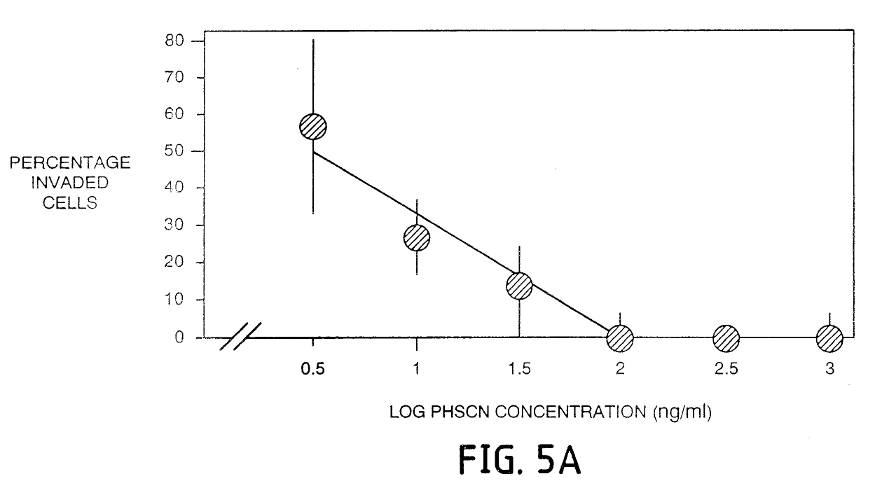

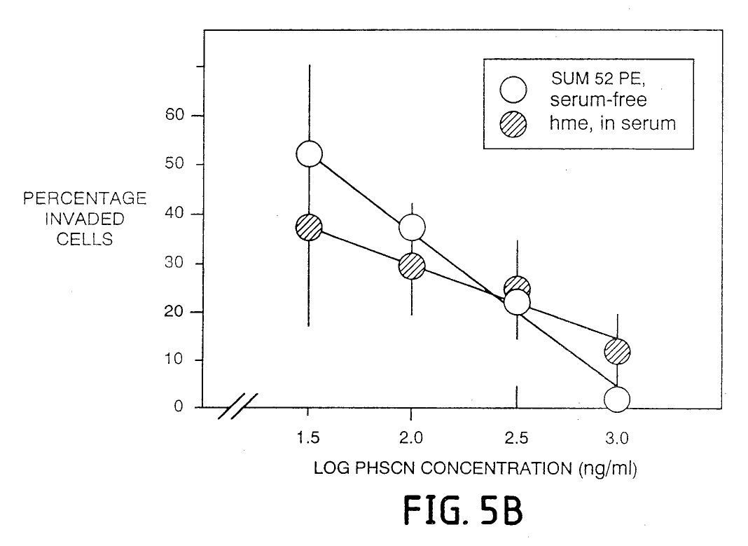

The results of adding varying concentrations of the PHSCN (SEQ ID NO:86) peptide to serum-induced metastatic SUM-52 PE breast cancer cells are presented in FIG. 5A. The logs of the PHSCN (SEQ ID NO:86) peptide concentrations in ng per ml are plotted on the X axis. The percentages of invaded SUM 52 PE cells relative to the percentage invaded in the absence of the PHSCN (SEQ ID NO:86) peptide are plotted on the Y axis. Mean invasion percentages are shown with their first standard deviations. Clearly, the PHSCN (SEQ ID NO:86) peptide exhibits a substantial inhibitory affect on these cells, even at relatively low concentrations. The PHSCN (SEQ ID NO:86) peptide's inhibitory affect is further demonstrated by the fact that relatively high concentrations cause complete inhibition.

The results of adding varying concentrations of the PHSCN (SEQ ID NO:86) peptide to PHSRN-induced (SEQ ID NO:1) metastatic SUM-52 PE breast cancer cells (in serum free medium) and normal human mammary epithelial cells (in 10% FCS), are presented in FIG. 5B. All invasion assays were carried out in 100 ng per ml of the PHSRN (SEQ ID NO:1) peptide to induce invasion. Again, the PHSCN (SEQ ID NO:86) peptide exhibits a substantial inhibitory affect on both cell lines at low concentrations, and almost complete inhibition at higher concentrations.

This example demonstrates the PHSCN (SEQ ID NO:86) peptide is an effective inhibitor of human breast cancer cell invasion. In this manner, the PHSCN (SEQ ID NO:86) peptide, or related sequences, are likely to provide effective therapy for human breast cancer by preventing the lethal affects of tumor cell metastasis.

### EXAMPLE 9

### Inhibiting Invasion Of Human Prostate Cancer Cells

In this example, the role of the PHSCN (SEQ ID NO:86) peptide in inhibiting the invasive behavior of metastatic prostate cancer cells is demonstrated. The method of Example 4 is employed, with the addition of varying concentrations of the PHSCN (SEQ ID NO:86) peptide.

Example 4 indicates that DU-145 cells are induced to invade the SU-ECM substrate in the presence of serum fibronectin or just the PHSRN (SEQ ID NO:1) sequence of fibronectin. Thus, the procedure in Example 4 provides a method for determining the inhibitory potential of the PHSCN (SEQ ID NO:86) peptide by comparing the number of cell invasions in the presence of the PHSCN (SEQ ID NO:86) peptide, with the number of cell invasions in the absence of the PHSCN (SEQ ID NO:86) peptide.

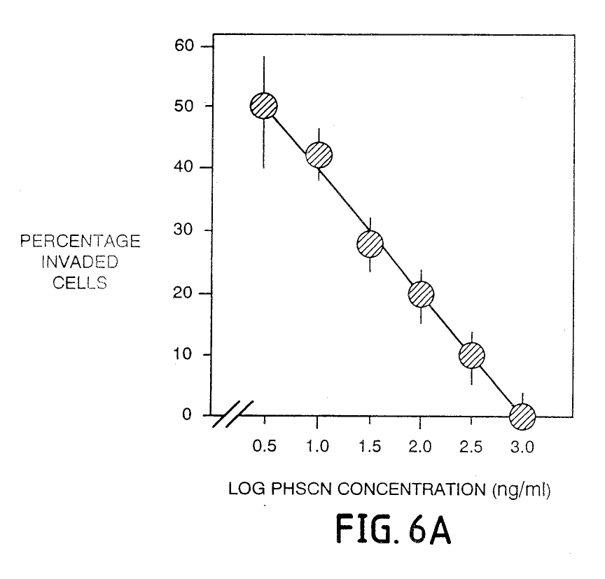

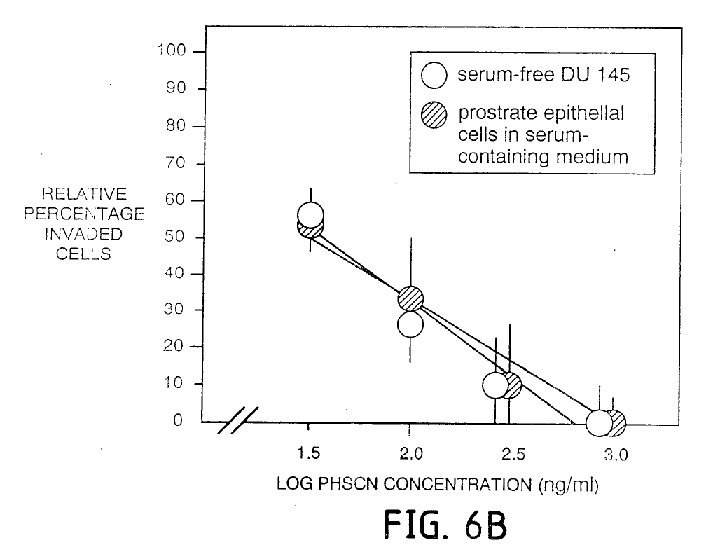

The results of adding varying concentrations of the PHSCN (SEQ ID NO:86) peptide to serum-induced invasion of metastatic DU-145 prostate cancer cells (in 10% serum) are presented in FIG. 6A. The logs of the PHSCN (SEQ ID NO:86) concentrations are plotted on the X axis. The percentages of invaded DU-145 cells relative to the percentage invaded in the absence of the PHSCN (SEQ ID NO:86) peptide are plotted on the Y axis. Mean invasion percentages are shown with their first standard deviations. Clearly, the PHSCN (SEQ ID NO:86) peptide exhibits a substantial inhibitory affect on these cells, even at relatively low concentrations. The PHSCN (SEQ ID NO:86) peptide's inhibitory affect is further demonstrated by the fact that relatively high concentrations cause complete inhibition.

The results of adding varying concentrations of the PHSCN (SEQ ID NO:86) peptide to PHSRN-induced (SEQ ID NO:1) metastatic DU-145 prostate cancer cells (in serum-free medium) and normal human prostate epithelial cells (in 10% FCS), are presented in FIG. 6B. All invasion assays were carried out in 100 ng per ml of the PHSRN (SEQ ID NO:1) peptide to induce invasion. Again, the results show that the PHSCN (SEQ ID NO:86) peptide exhibits a substantial inhibitory affect on both cell lines at low concentrations, and almost complete inhibition at higher concentrations.

This example demonstrates the PHSCN (SEQ ID NO:86) peptide is an effective inhibitor of human prostate cancer cell invasion. In this manner, the PHSCN (SEQ ID NO:86) peptide may provide an effective therapy for human prostate cancer by preventing the lethal affects of tumor cell metastasis.

### EXAMPLE 10

### Inhibiting Invasion of Rat Prostate Cancer Cells

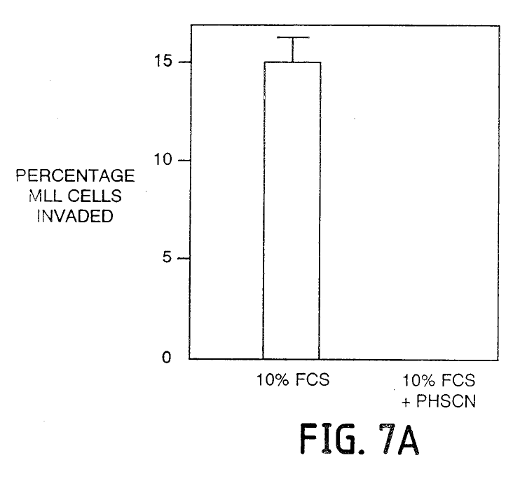

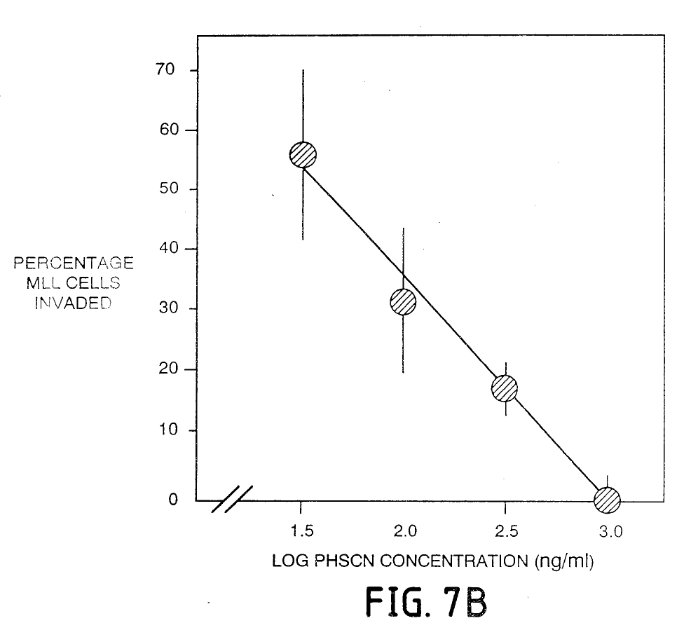

In this example, the role of the PHSCN (SEQ ID NO:86) peptide in inhibiting the invasive behavior of rat metastatic prostate carcinoma MatLyLu (MLL) cells is demonstrated (see Example 4 for the general procedure employed). The result of adding 1 microgram per ml of the PHSCN (SEQ ID NO:86) peptide to serum-induced MLL cells causes complete inhibition of invasion (see FIG. 7A).

The result of adding a varying concentration of the PHSCN (SEQ ID NO:86) peptide to PHSRN-induced (SEQ ID NO:1) MLL cells in serum free media is shown in FIG. 7B, where 100 ng per ml of PHSRN (SEQ ID NO:1) was used to induce invasion. FIG. 7B indicates that the PHSCN (SEQ ID NO:86) peptide exhibits a substantial inhibitory affect even at low concentrations, and almost complete inhibition at higher concentrations. This example demonstrates invasion of rat prostate cancer cells is inhibited in the same manner as human breast cancer cells (see Example 8) and human prostate cancer cells (see Example 9).

### EXAMPLE 11

### Inhibiting Invasion of Rat Prostate Cancer Cells

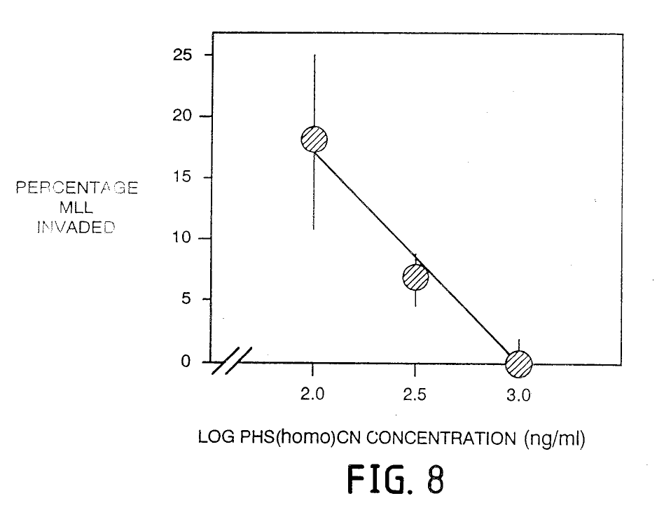

In this example, the role of a homo-cysteine containing peptide (i.e., PHS(hC)N) (SEQ ID NO:85) in inhibiting the invasive behavior of rat metastatic prostate carcinoma MatLyLu (MLL) cells is demonstrated. The procedure described in Example 10, was employed using SU-ECM substrates in 10% FCS and PHS(hC)N (SEQ ID NO:85) instead of PHSCN (SEQ ID NO:86). The result of adding varying concentrations of the PHS(hC)N (SEQ ID NO:85) peptide to serum-induced MLL cells indicates this peptide also has an inhibitory affect on cell invasion (see FIG. 8). As with the PHSCN (SEQ ID NO:86) peptide, the PHS(hC)N (SEQ ID NO:85) peptide substantially inhibits invasion at lower concentrations, and completely inhibits invasion at higher concentrations. This example demonstrates that the PHS(hC)N (SEQ ID NO:85) peptide has a similar inhibitory affect as the PHSCN (SEQ ID NO:86) peptide.

### EXAMPLE 12

### Inhibiting Growth and Metastasis of Prostate Cancer Tumors In Vivo

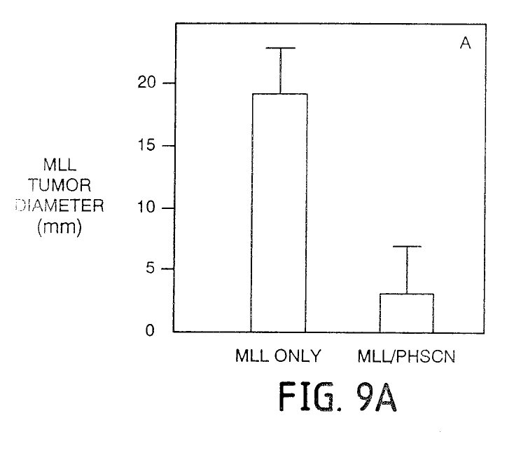

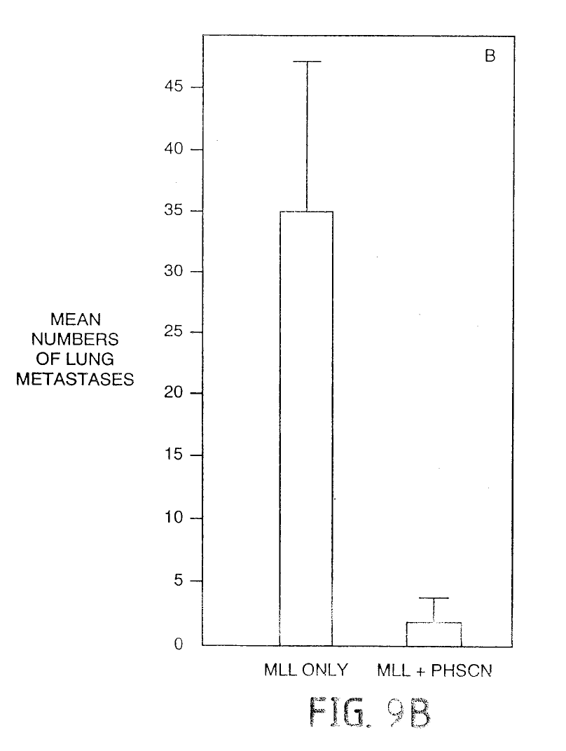

In this example, the role of the PHSCN (SEQ ID NO:86) peptide in inhibiting the growth and metastasis of prostate cancer tumors in vivo is demonstrated. In the first part of this example, four Copenhagen rats were injected with 500,000 MatLyLu (MLL) cells subcutaneously in the thigh. Two of these rats also received 1 mg of the PHSCN (SEQ ID NO:86) peptide along with the injected MLL cells, and thereafter received 1 mg of the PHSCN (SEQ ID NO:86) peptide injected in their tail vein three time per week for two weeks. The other two injected rats were left untreated. Tumor sizes were measured with calipers on day 14, and the tumors in the untreated rats were removed. The results depicted in FIG. 9A, clearly demonstrate that the PHSCN (SEQ ID NO:86) peptide significantly slows the growth of injected MLL tumors in rats. It is possible that the ability of the PHSCN (SEQ ID NO:86) peptide to slow tumor growth is due to its inhibition of tumor invasion by normal endothelial cells, an anti-angiogenic effect.

Two weeks after the size of the tumors were measured, the rats were sacrificed and the mean number of lung metastases was determined at 10-fold magnification. The mean number of lung metastases in the untreated mice (MLL only) was nearly 35 in spite of the fact that the initial prostate tumors had been removed when their size was measured. The mean number of lung metastases in the treated mice (MLL+PHSCN (SEQ ID NO:86)) was less than 5, even though the initial prostate tumors were never removed because they were too small. This striking difference in mean number of metastases, depicted in FIG. 9B, indicates that the PHSCN (SEQ ID NO:86) peptide significantly inhibits tumor cell metastasis in rats. In this manner, the PHSCN (SEQ ID NO:86) peptide provides effective in vivo therapy for cancer by preventing the lethal effects of tumor cell growth and metastasis.

### EXAMPLE 13

### Inhibiting Growth and Metastasis of Prostate Cancer In Vivo

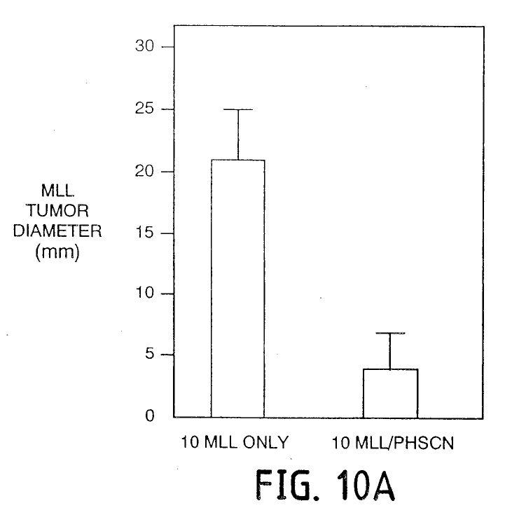

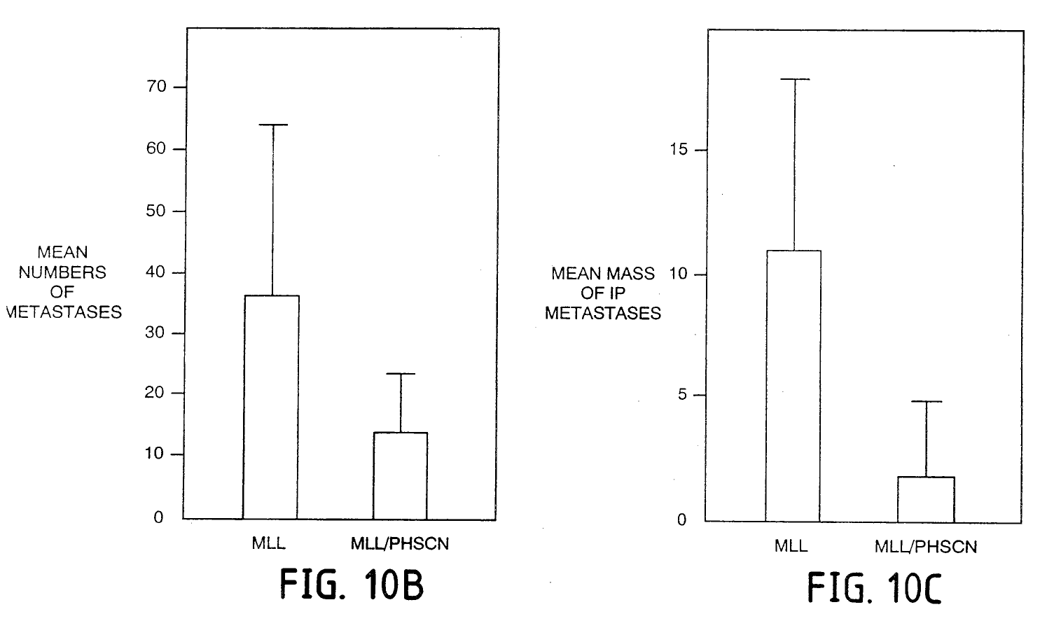

In this example, as in Example 12, the role of the PHSCN (SEQ ID NO:86) peptide in inhibiting the growth and metastasis of prostate cancer tumors in vivo is demonstrated. In the first part of this example, 20 Copenhagen rats were injected with 500,000 MatLyLu (MLL) cells subcutaneously in the thigh. To more closely approximate a real clinical situation, PHSCN (SEQ ID NO:86) peptide treatment of 10 of these rats was initiated after 24 hours, instead of immediately. The 10 treated rats (MLL/PHSCN: SEQ ID NO:86) received a total of 5 i.v. injections of 1 mg of the PHSCN (SEQ ID NO:86) peptide through the tail vein over two weeks. Tumor sizes were measured with calipers on day 14, and the tumors in the untreated rats were removed. Since the injected tumors in the MLL/PHSCN (SEQ ID NO:86) rats were still small, they were retained in the rats for another 7 to 9 days following the last PHSCN (SEQ ID NO:86) injection. At this time, their sizes were all greater than 2 cm, and they were also removed. The result of the first part of this example, depicted in FIG. 10A, clearly indicates that the PHSCN (SEQ ID NO:86) peptide, even when administered after the tumor cells have “seeded”, substantially slows the growth of rat prostate cancer tumors.

The dramatic growth-inhibitory effect of the PHSCN (SEQ ID NO:86) peptide on MLL tumors may be due to their inhibition of the invasion of host endothelial cells into the tumor. Host endothelial cell invasion may be induced by the secretion of large amounts of proteinases from the tumors, and the resulting fragmentation of host plasma fibronectin. Fibronectin fragments have been shown to stimulate the migratory/invasive behaviors of normal mesenchymal and endothelial cells. This angiogenic process is believed to occur during normal wound healing. Thus, the ability of metastatic cells to be constitutively induced by intact plasma fibronectin to express proteinases and invade may play a central role both in tumor cell invasion and in tumor growth. In this manner, the PHSCN (SEQ ID NO:86) peptide is an effective chemotherapeutic to prevent the growth of tumors in vivo.

In the second part of this example, the MLL/PHSCN (SEQ ID NO:86) rats received 2 more i.v. doses of the PHSCN (SEQ ID NO:86) peptide prior to sacrifice. Ten days after the sizes of the injected primary tumors were determined, all the rats in the two groups (MLL only and MLL/PHSCN (SEQ ID NO:86)) were sacrificed, and the number of lung metastases was determined at 7.5-fold magnification. As can be seen in FIG. 10B, there is a significant reduction in the mean numbers of lung metastases in the rats which received PHSCN (SEQ ID NO:86) treatment as compared to the untreated rats.

The 20 rats described in parts one and two of this example were also examined for metastatic tissues in their lymphatic systems. All of these metastases were dissected and weighed. FIG. 10C plots the mean masses of intraperitoneal metastases (grams) for the two groups of 10 rats. As is clearly demonstrated, there is a significant reduction in the mean masses of lymphatic metastases in the rats which received PHSCN (SEQ ID NO:86) peptide treatment, as compared to the untreated rats. This may be due to the anti-angiogenic effect of the PHSCN (SEQ ID NO:86) peptide, as described in part one of this example. In this manner, the PHSCN (SEQ ID NO:86) peptide maybe an effective anti-metastatic, growth-inhibiting chemotherapeutic agent for use in the treatment of cancer.

From the above, it should be clear that the present invention provides an anticancer approach that is reliable for a wide variety of tumor types, and particularly suitable for invasive tumors. Importantly, the treatment is effective with minimal host toxicity.

### EXAMPLE 14

### Synthesis of PHSCN (SEQ ID NO:86)-Polylysine Dendrimers

This example describes one embodiment of a synthetic pathway to create a variety of peptide dendrimers. Specifically, PHSCN (SEQ ID NO:86) peptides, substituted to branched polylysine dendrimers of different sizes, are synthesized.

Branched polylysine cores, each with 4, 8, or 16 sites for peptide attachment, are commercially obtained (i.e., for example, Novabiochem/EMD Biosciences, San Diego Calif.; or VivaGel®, Starphamia, Melbourne, Australia or obtained from the CORE facility at the University of Michigan). Then, the PHSCN (SEQ ID NO:86), PHSCNGG (SEQ ID NO:108), PHSCNGGK (SEQ ID NO:105), or HSPNC (SEQ ID NO:107) peptides described above are covalently attached to the dendrimer cores, using traditional solid phase peptide synthesis and employing current methods known in the art regarding F-moc chemistry (supra). Ambulos, N. Analysis of Synthetic Peptides. In: Solid Phase Synthesis, Kates, S. A., Albericio, F., eds. Marcell Dekker, Inc., New York., pp. 782-805 (2002).

After dendrimer-peptide synthesis, the peptides are analyzed by high pressure liquid chromatography-mass spectrometry (HPLC-MS) using traditional methods known in the art. Specifically, the dendrimer-peptides are isolated by HPLC and then identified by MS. Amino acid sequence analysis (i.e., using, for example, Edman degradation) confirmed the correct peptide-dendrimer composition.

### EXAMPLE 15

### Comparative FAK/PI3′K/Akt Inhibition Potency in Relation to Various PHSCN Dendrimers

This example will compare the efficacy of a series of branched polylysine dendrimers having an increasing number PHSCN (SEQ ID NO:86)-peptides on a variety of biochemical pathways. This experiment will investigate the relative efficacy of PHSCN-substituted dendrimers having higher numbers of attached PHSCN-related peptides for FAK/PI3′K/Akt pathway inhibition. Specifically, this experiment will investigate whether PHSCN (SEQ ID NO:86)-substituted dendrimers having higher numbers of attached PHSCN (SEQ ID NO:86) peptides might be more effective at inducing apoptosis in cultured DU 145 cells.

First, the potencies of several Ac-PHSCN-substituted, branched polylysine dendrimers will be compared to soluble Ac-PHSCN-NH, monomer peptides at a series of concentrations. These Ac-PHSCN-dendrimers are varied systematically in regards to the number of attached PHSCN peptides. Branched, polylysine dendrimer cores will be used comprising either 4, 8, or 16 attached Ac-PHSCN (SEQ ID NO:86) peptides. Second, an Ac-HSPNC-substituted (SEQ ID NO:107) dendrimer will be utilized as a negative control (i.e., for example, a scrambled control peptide).

The peptide-dendrimers will be incubated in adherent DU 145 cells cultures in serum-containing medium. Subsequently, dose response data that compare the PHSCN-dendrimers and PHSCN monomer potencies will be generated using the following techniques: i) immunoblotting; ii) co-immunoprecipitation; iii) cell counting with phase-contrast microscopy; and iv) immunohistochemistry.

The data is expected to show that PHSCN-dendrimers: i) inhibit specific steps in the FAK/PI3′K/Akt pathway; ii) decrease adherent DU 145 cell numbers; and iii) induce cellular apoptosis. Although it is not necessary to understand an invention, it is believed that PHSCN-dendrimers elicit their effects in at least one of the following ways:
a) inhibiting serum-induced FAK phosphorylation at Y397;
b) inhibiting serum-induced association of the PI3′-kinase p85 regulatory subunit with FAK;
c) inhibiting serum-induced Akt phosphorylation at S473;
d) decreasing the growth of adherent DU 145 cells, in the presence of serum;
e) upregulating Bad and Bax protein expression in whole or fractionated cell lysates;
f) inducing Caspase activation, especially Caspase 9, Caspase 3, and Caspase 6; or
g) inducing the cytokeratin 18 epitope by immunohistochemistry in fixed, adherent cells.

### EXAMPLE 16

### Comparative FAK/PI3K/Akt Inhibition Potencies of Optimally Branched PHSCN Dendrimers

This example will compare the potencies between Ac-PHSCN (SEQ ID NO:86)-, Ac-PHSCN GG- (SEQ ID NO:108), Ac-PHSCNGGK- (SEQ ID NO:105), Ac-PHSCN-GGK(biotin) and Ac-PHSCNGGK(fluorescein)-substituted dendrimers at their most efficacious polylysine branching number.

The optimal branch number of polylysine dendrimers substituted to PHSCN (SEQ ID NO:86)-peptides are selected according to Example 15. The efficacy of these five peptide-dendrimers will be compared by constructing individual dose response curves using the same techniques as described in Example 15.

### EXAMPLE 17

### Comparative Potencies of Phscn-Dendrimers to Inhibit Cancer Cell Invasion

This example constructs a dose-response curve for a dendrimer substituted with 8-substituted Ac-PHSCN dendrimers to inhibit in vitro DU145 prostrate cancer cell invasion induced by 10% serum (i.e., containing fibronectin comprising SEQ ID NO:1). The data is compared to the inhibition potencies of the previously reported soluble Ac-PHSCN-NH₂ monomer peptide. Livant et al., “The PHSCN Sequence As An Anti-Invasive For Human Prostate Carcinoma Cells, And As An Anti-Tumorigenic And Anti-Metastatic Agent For Rat Prostate Cancer” Cancer Research 60: 309-320 (2000); and Livant et al., “The PHSRN Sequence Induces Extracellular Matrix Invasion And Accelerates Wound Healing In Obese Diabetic Mice” J. Clin. Invest. 105:1537-1545 (2000).

In this experiment acetylated PHSCN peptide (Ac-PHSCN) was attached to polylysine dendrimers, thereby creating a PHSCN-substituted dendrimer. Although it is not necessary to understand the mechanism of an invention, it is believed that polylysine dendrimers utilize the NH₂ groups of the lysine N-terminus. Further, the available lysine side chains then to attach the C-termini of other lysines. The result forms a “branching tree” comprising lysines joined by amide linkages. In this example, the dendrimers had 8 NH₂ groups available (on 4 lysine residues) for PHSCN attachment. See FIG. 23. Thus, in PHSCN dendrimers, the C-terminus of each Ac-PHSCN peptide was attached to a lysine NH₂ group via an amide linkage. The N-terminus of each attached Ac-PHSCN peptide was acetylated.

Naturally serum-free, selectively permeable, sea urchin embryo basement membranes were prepared by the detergent (Triton®) treatment of sea urchin embryos, cultured for 72 hours in sea water. Permanent salt water aquariums were maintained that contain gravid adult sea urchins, so that basement membranes were always available. In vitro invasion assays using sea urchin embryo basement membranes were performed as previously reported. Livant et al., “Methods And Compositions For Wound Healing” U.S. Pat. No. 6,576,440 (2003) (herein incorporated by reference); and Livant et al., “Invasion of Selectively Permeable Sea Urchin Embryo Basement Membranes by Metastatic Tumor Cells, But Not By Their Normal Counterparts” Cancer Research 55, 5085-5093 (1995).

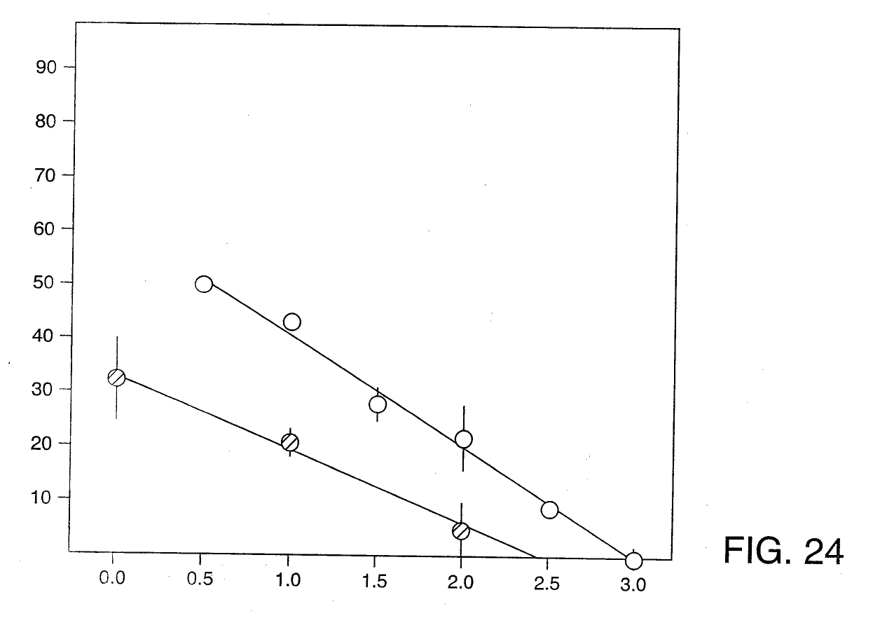

The results show that the 8-substituted Ac-PHSCN dendrimers were at least 10-fold more potent than the Ac-PHSCN-NH2 monomer at inhibiting serum-induced invasion by DU 145 metastatic human prostate cancer cells. See FIG. 24. Each point was determined in triplicate, shown with the first standard deviation. Invasion percentages were expressed relative to the percentage of DU 145 cells invaded in the absence of an Ac-PHSCN dendrimer or in the presence of a PHSCN-dendrimer scrambled peptide control (i.e., for example, Ac-HSPNC).

Although it is not necessary to understand the mechanism of an invention, previous data indicated that the PHSCN (SEQ ID NO:86) peptide might interact with the integrin β₁ subunit of α5β₁ to inhibit both invasion and survival. It is further believed that the present data suggests that PHSCN-substituted dendrimers, may also be especially potent invasion-inhibitory agents, acting by the same mechanism.

### EXAMPLE 18

### In Vivo Antitumorigenesis in Nude Mice

This example will demonstrate the ability of PHSCN (SEQ ID NO:86)-dendrimers to reduce, in vivo, human tumorigenesis and metastasis in athymic, nude mice. These experiments will determine whether PHSCN-dendrimer therapy results in increased numbers of apoptotic cells in DU 145 tumors, relative to therapy with the Ac-PHSCN-NH₂ peptide monomer or with the corresponding HSPNC-dendrimer negative control. These protocols have been approved by the University of Michigan Institutional Animal Care And Use Committee (Approval # 8630, Renewal # 7608).

Nude mice will be treated systemically via thrice-weekly Ac-PHSCN-NH₂ monomer peptide (0.1-10 mg/kg) intravenous injection. Also, Ac-PHSCNGGK(biotin)-(SEQ ID NO:105) or Ac-PHSCNGGK (fluorescein) (SEQ ID NO:108)- substituted dendrimers will be tested in these mice to determine their localization in tumor cells and associated vasculature. These studies involve traditional immunohistochemistry techniques using fluorescent secondary antibody (biotin-substituted dendrimers), or by fluorescent microscopy (fluorescein-substituted dendrimers). Although it is not necessary to understand the invention, it is believed that the PHSCN (SEQ ID NO:86) peptides account for the majority of the dendrimer molecular weight such that the most effective in vivo dosages is not likely to be more, and may be substantially less, than the most effective monomer PHSCN-peptide dosage (i.e., for example, 5 mg/kg).

Detection of apoptotic cells at various stages in the growth of DU 145 primary tumors will occur by the following procedure: i) tissue fixation; ii) paraffin-embedding; iii) tissue slice sectioning; and iv) staining tumor cells and surrounding non-tumor tissue.

We will utilize commercially available apoptosis detection kits to detect DNA cleavage in fixed tissues (i.e., for example, DNA Laddering Kits, R&D Systems, Minneapolis, Minn.), as well as commercially available immunohistochemical kits to detect Bax upregulation (i.e., for example, Mouse Bax MAb (Clone YTH5B7); #2280-MC-100; R&D Systems, Minneapolis, Minn.). Immunohistochemical staining will also be performed to detect the presence of the M30 cytokeratin 18 epitope. Leers et al., “An Immunohistochemical Study Of The Clearance Of Apoptotic Cellular Fragments” Cell Mol. Life. Sci. 59:1358-1365 (2000).

The data will show that PHSCN-substituted dendrimers are more potent antitumorigenic and antimetastatic therapeutic agents in nude mice bearing DU 145 tumors than the soluble monomer PHSCN (SEQ ID NO:86) peptide or the HSPNC-dendrimer negative control (SEQ ID NO:107). These data should also show that the inhibitory potencies of PHSCN (SEQ ID NO:86)-substituted dendrimers are parallel to those determined above for the in vitro biochemical pathways (Examples 15 and 16) and on α5β₁-mediated in vitro tumor cell invasion (Example 17).

### EXAMPLE 19

### Prostate Cancer Cell Viability

This example presents data showing DU 145 prostate cancer cell viability in the presence of PHSCN-dendrimers.

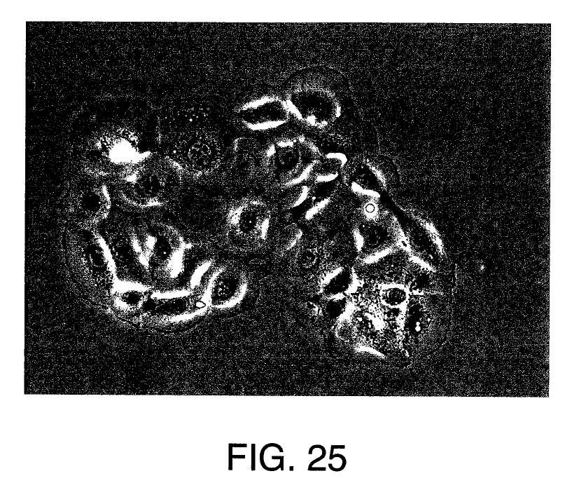

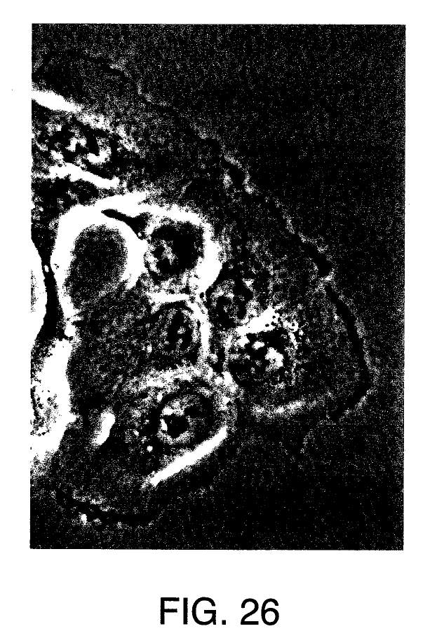

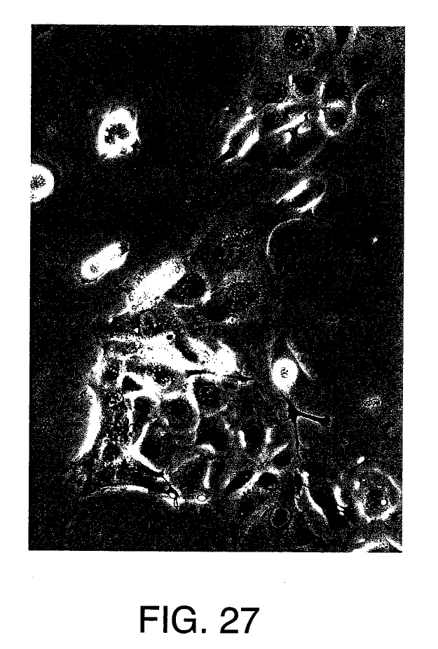

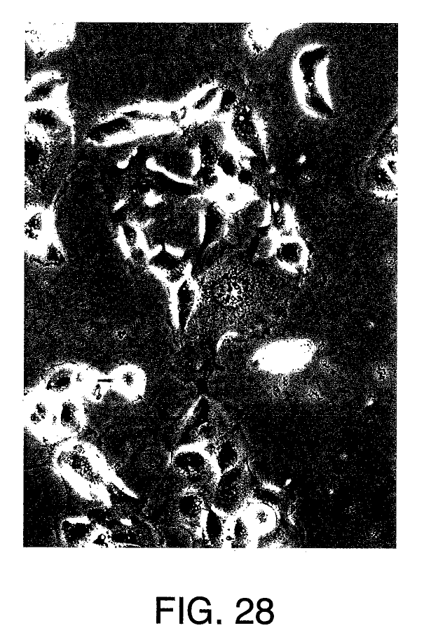

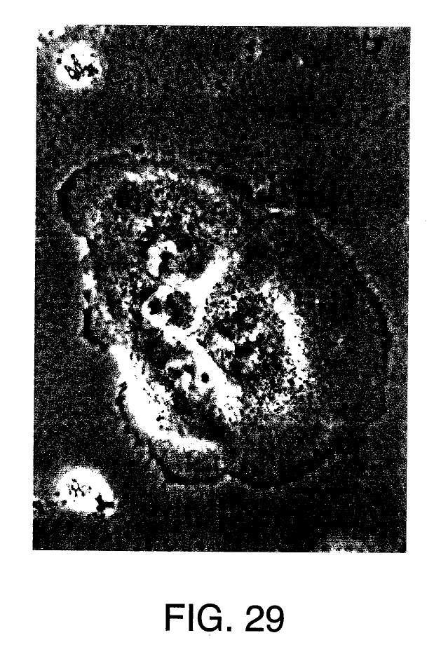

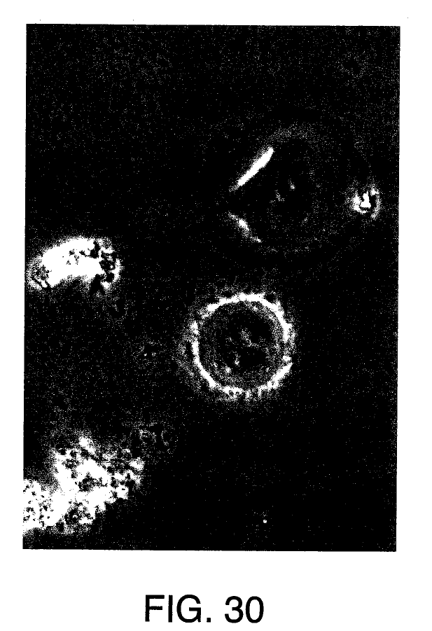

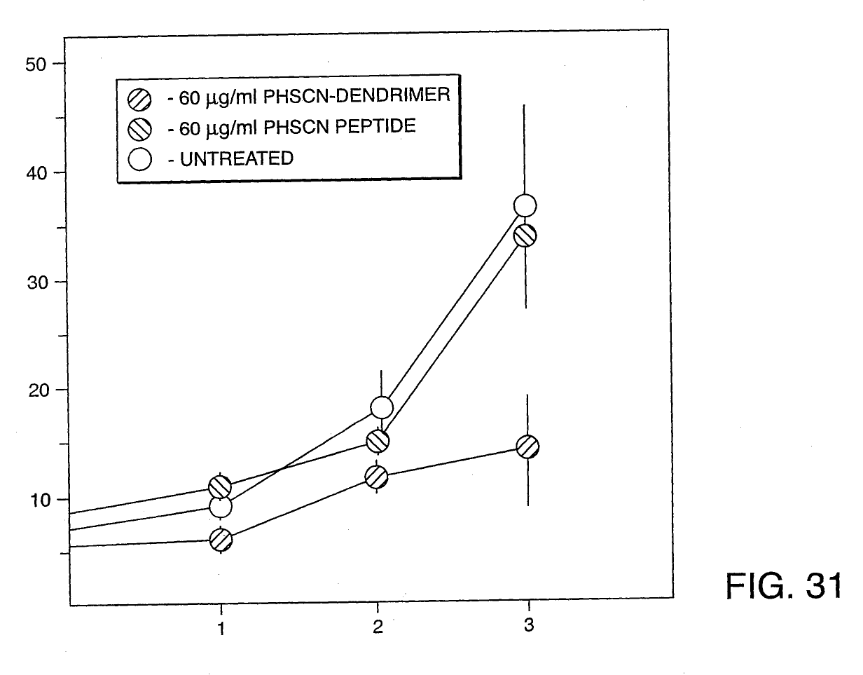

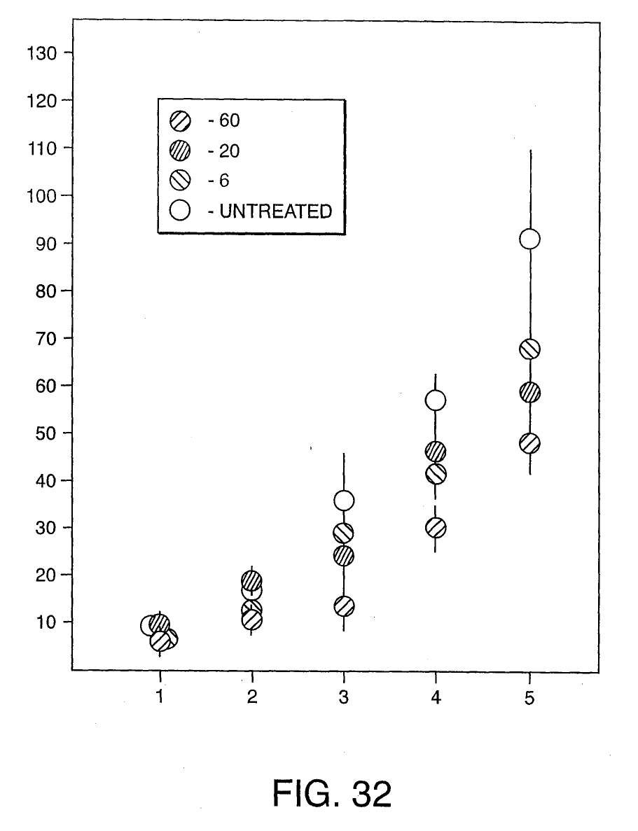

DU145 cells were grown in culture in accordance with the methods described in Example 17. After a 24 hour incubation with 8-PHSCN dendrimers (60 μg/ml/20,000 cells), approximately 50% adherent DU 145 cells were killed. Untreated DU 145 cells are shown in FIG. 25 (200-fold magnification) and FIG. 26 (630-fold magnification). These untreated DU 145 cells were nicely spread having intact cell membranes and no intracellular debris outside of the cells were observed. Treated DU 145 are shown in FIG. 27 & FIG. 28 (both 200-fold magnification), and FIG. 29 & FIG. 30 (both 630-fold magnification). These PHSCN-dendrimer treated DU 145 cells have many cytoplasmic granules that have leaked outside of the cells. These cytoplasmic granules may be ribosomes, or may be membrane-enclosed vesicles generated during autophagy that were shed from the cells as they entered necrosis. Further, the general cell appearance is characteristic of cells undergoing death by necrosis. Specifically, the PHSCN-dendrimer treated cells were swollen, had abnormal protrusions, and had damaged cell membranes. Dead cells were also present among the extracelluar debris field. These attributes are also characteristic of cells undergoing necrosis.

### EXAMPLE 20

### In Vivo Antitumorigenesis In Copenhagen Rats

This example compares the effects of intravenously administered PHSCN peptide (Ac-PHSCN-NH2) with an 8-substituted Ac-PHSCN dendrimer on MATLyLu rat prostate cancer tumorigenesis in Copenhagen rats.

Thirty-two (32) rats were injected with 100,000 MATLyLu cells (i.m., right flank). Beginning 24 hours after tumor cell injection tail vein injection of PHSCN-comprising peptides (1 mg in 100 μl normal saline per rat) was performed three times per week for two weeks according to the following experimental design: i) Ac-PHSCN-NH₂ monomer peptide (10 rats); ii) 8-substituted Ac-PHSCN dendrimers (10); and iii) normal saline controls (12).

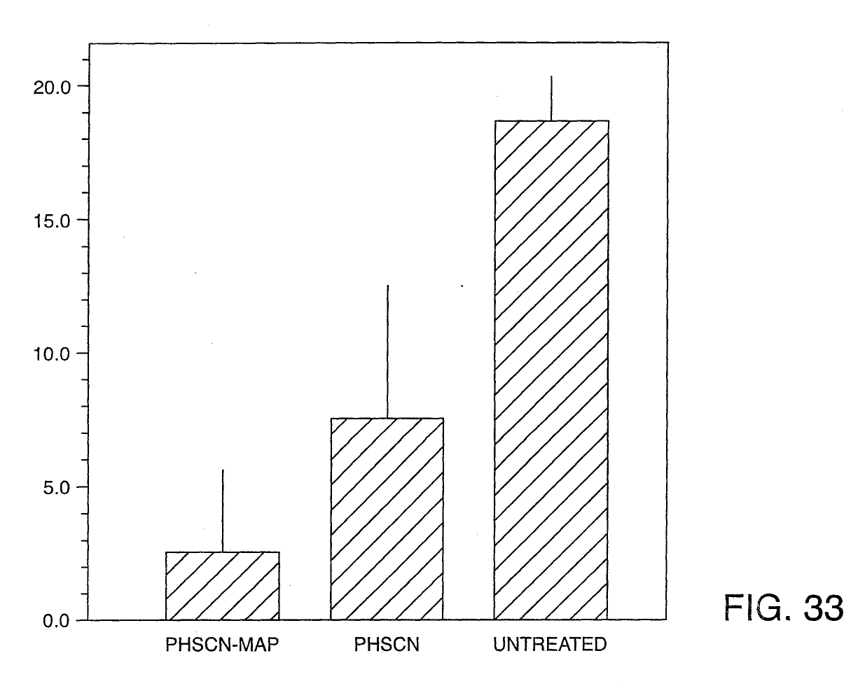

After the two week treatment period, the rats were euthanized and the tumor diameters were measured in millimeters. The data shows that 8-substituted Ac-PHSCN dendrimer reduced tumor diameter by almost a factor of ten when compared to the saline injected controls. Additionally, the 8-substituted Ac-PHSCN dendrimer reduced tumor diameter between 3-4 fold greater than did an identical dosage/dosage schedule of the Ac-PHSCN-NH₂ peptide monomer (See FIG. 33).

### EXAMPLE 21

### Inhibition of DU145 Prostate Cancer Cell Growth

This example demonstrates that 8-substituted PHSCN dendrimers inhibit DU145 cancer cell growth at a much greater potency than the Ac-PHSCN-NH₂ monomer peptide.

DU145 cells were grown in a 10% serum culture in accordance with the methods described in Example 17. The data shown in FIG. 25 clearly demonstrate that 8-substituted PHSCN dendrimers inhibit DU145 cell growth at three-times the potency of the Ac-PHSCN-NH₂ monomer peptide after a three day incubation period. A dose response relationship for the 8-substituted PHSCN dendrimer is presented in FIG. 26. Over a five day period, the relative inhibition percentage by 60 μg/ml 8-substituted PHSCN dendrimer increased to approximately 200% relative to the untreated control. Also, evident in FIG. 26 is the increasing relative inhibition percentages between the 6, 20 & 60 μg/ml 8-substituted PHSCH dendrimer concentrations.

### EXAMPLE 22

### SU-ECM Invastion Inhibition Using Pancreatic Cancer Cell Culture Models

This example demonstrates that a PHSCN-dendrimer is more potent at inhibiting in vitro invasion than the soluble PHSCN monomer when using BxPC-3 as a pancreatic cancer cell line.

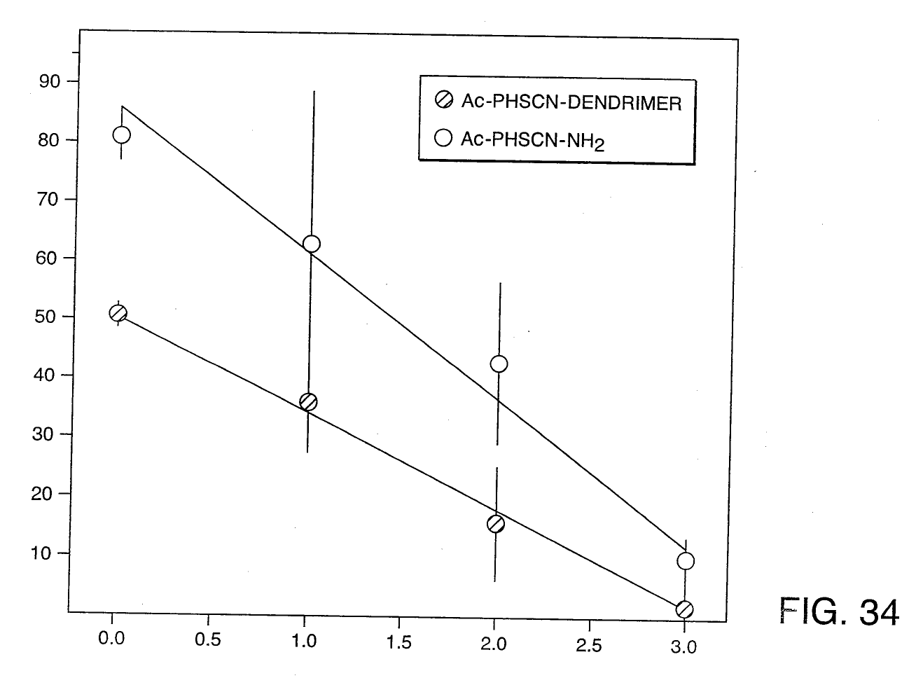

This example was performed in accordance with Example 17 with the exception that BxPC-3 pancreatic cancer cells were substituted for DU145 prostate cancer cells. As shown in FIG. 34,

the 8-substituted PHSCN-dendrimer is at least 20-fold more potent an invasion inhibitor for 10% serum-induced invasion than the Ac-PHSCN-NH₂ monomer (See FIG. 34). These data demonstrate that an 8-substituted Ac-PHSCN dendrimer significantly induces greater adherent BxPC-3 cell death than the Ac-PHSCN-NH₂ peptide monomer.

### EXAMPLE 23

### SU-ECM Invasion Inhibition Using Pancreatic Cancer Models

This example will utilize SU-ECM basement membranes to compare the invasion-inhibitory potencies of Ac-PHSCN-substituted, branched polylysine dendrimers with soluble Ac-PHSCN-NH₂ peptide, while systematically varying the numbers of attached PHSCN peptides on the dendrimers using a PANC-1 pancreatic cancer cell line. Further, immunoblotting and assays of enzymatic activity investigate the relative potencies of PHSCN-dendrimers and monomeric Ac-PHSCN-NH₂ peptide on MMP-1 expression.

Specifically, PANC-1 cells will be suspended on the surfaces of SU-ECM basement membranes in accordance with Example 17 in order to compare the invasion-inhibitory potencies of Ac-PHSCN-dendrimer with the Ac-PHSCN-NH₂ monomer peptide, and the appropriate HSPNC scrambled peptide negative control. Branching PHSCN-dendrimers with varying multiplicities of 4, 8, and 16 PHSCN ligands will be compared. Additionally, as it is known that interstitial collagenase MMP-1 is crucial for serum-induced invasion by metastatic human breast cancer cell lines, MMP-1 expression will be investigated by immunoblotting, coimmunoprecipitation, and commercially available quantitative MMP-1 assays. Jia et al., “Integrin Fibronectin Receptors In MMP-1 Dependent Invasion By Breast Cancer And Mammary Epithelial Cells” Cancer Research, in press (2004).

### EXAMPLE 24

### FAK/PI3′K/Akt Pathway Inhibition in Pancreatic Cancer Models

This example investigates whether PHSCN-substituted dendrimers are more potent inhibitors of the FAK/PI3′K/Akt pathway than the Ac-PHSCN-NH₂ monomer. Cultured BxPC-3, AsPC-1, or Capan-1 cells, like breast and prostate carcinoma cell lines are known to express α5β₁ but not α4β₁ integrin fibronectin receptors. Livant et al., “The PHSCN Sequence As An Anti-Invasive For Human Prostate Carcinoma Cells, And As An Anti-Tumorigenic And Anti-Metastatic Agent For Rat Prostate Cancer” Cancer Research 60:309-320 (2000); Ignatoski et al., “p38 MAPK Induces Cell Surface α4 Integrin Down-Regulation to Facilitate erbB-2 Mediated Invasion” Neoplasia 5(2):128-134 (2003); and Lohr et al., “Expression And Function Of Receptors For Extracellular Matrix Proteins In Human Ductal Adenocarcinomas Of The Pancreas” Pancreas 12:248-259 (1996). Consequently, this example will utilize these cells as a model system to compare serum-induced invasion inhibition and apoptosis induction by PHSCN-comprising peptides.

Thus, the apoptosis-inducing potencies of Ac-PHSCN-substituted, branched polylysine dendrimers to soluble Ac-PHSCN-NH₂ peptide will be compared by utilizing cultures of adherent BxPC-3, AsPC-1, or Capan-1 cells in serum-containing medium. Additionally, the relative potencies of various PHSCN dendrimer peptides will be compared by systematically varying the numbers of attached PHSCN peptides on the dendrimers. A scrambled peptide (i.e., for example, an Ac-HSPNC-substituted dendrimers will serve as a negative control. The techniques of immunoblotting, coimmunoprecipitation, cell counting with phase-contrast microscopy, flow cytometry, immunohistochemistry, and enzymatic assays for DNA fragmentation will be used to compare the dose responses of the PHSCN-dendrimers and PHSCN monomers for inhibiting specific steps in the FAK/PI3′-kinase/Akt pathway, decreasing the numbers of adherent BxPC-3, AsPC-1, or Capan-1 cells and inducing apoptosis.

### EXAMPLE 25

### In Vivo Inhibition of Pancreatic Adenocarcinoma Tumorigenesis

This example demonstrates that PHSCN-substituted dendrimers will be more potent antimetastatic agents in athymic nude mice bearing orthotopic BxPC-3, AsPC-1, or Capan-1 tumors than a monomeric Ac-PHSCN-NH₂ peptide. The most potent PHSCN-substituted dendrimer determined in accordance with Example 24, will be tested against an HSPNC-dendrimer (i.e., a negative control) and the Ac-PHSCN-NH₂ monomer peptide.

Human BxPC-3, AsPC-1, or Capan-1 human pancreatic adenocarcinoma cells will be surgically, orthotopically implanted and allowed to grow into primary pancreatic tumors. Then PHSCN peptides will be systemically administered a PHSCN-comprising peptide via thrice-weekly intravenous injection into athymic nude mice. Inhibition of primary pancreatic metastasis will then be assessed by anti-apoptotic effects of the PHSCN comprising peptides.

A comparison will be performed at several PHSCN-peptide dosage levels, including 5 and 50 mgs. Standard immunohistochemical techniques, enzymatic techniques, and commercially available kits will be employed to detect apoptotic cells at various stages in the growth of BxPC-3, AsPC-1, or Capan-1 primary tumors. Leers et al., “An Immunohistochemical Study Of The Clearance Of Apoptotic Cellular Fragments” Cell. Mol. Life. Sci. 59:1358-1365 (2002). Because of the numerous sites for PHSCN peptide attachment on dendrimers, PHSCN peptides will account for the majority of dendrimer molecular weight; thus, the dosages of dendrimers likely to be effective in vivo are not likely to be more, and may be substantially less, than the effective monomer PHSCN peptide dosage of 5 mg.

To compare the effects of systemic PHSCN peptide and PHSCN-dendrimer on pancreatic cancer metastasis, variants of BxPC-3, AsPC-1, or Capan-1 cells comprising a green fluorescence protein gene or the luciferase gene are injected that are fluorescent in vivo. Bouvet et al., “Real-Time Optical Imaging Of Primary Tumor Growth And Multiple Metastatic Events In A Pancreatic Cancer Orthotopic Model” Cancer Res. 62:1534-1540 (2002). Nude mice treated systemically with fluorescent Ac-PHSCN-NH₂, Ac-HSPNC-NH₂, Ac-PHSCN-dendrimer, or Ac-HSPNC-dendrimer will be scanned to quantitate levels of BxPC-3 and PANC-1 metastasis.

Standard immunohistochemical techniques, enzymatic techniques, and commercially available kits will be employed to detect apoptotic cells at various stages in the growth of BxPC-3, AsPC-1, or Capan-1 primary tumors. To detect apoptotic cells at various growth stages, primary tumors and surrounding host tissue will be fixed, paraffin-embedded, sectioned, and stained. Commercially available apoptosis detection kits will detect DNA cleavage in fixed tissues, as well as standard immunohistochemical methods with commercially available antibodies to detect BAX upregulation. Immunohistochemical staining will also be performed to detect the presence of the M30 cytokeratin 18 epitope. Livant et al., “Invasion Of Selectively Permeable Sea Urchin Embryo Basement Membranes By Metastatic Tumor Cells, But Not By Their Normal Counterparts” Cancer Research 55:5085-5093 (1995).

### EXAMPLE 26

### Proteomic Analysis of Treated DU 145 Cells

This experiment will compare the expressed protein patterns of DU 145 cells either treated with Ac-PHSCN dendrimers, Ac-PHSCN-NH₂ monomer peptide, or an untreated control.

DU145 cells were grown in culture in accordance with the methods described in Example 17. Following incubation with the appropriate peptide, the cells will be lysed, proteins extracted and placed on a high-resolution 2D gel electrophoresis system. The two thousand (2,000) most prevelant proteins expressed by each treatment group of cells will be evaluated.

1. A macromolecule comprising a dendrimer, said dendrimer comprising branches attached to a plurality of peptides, each of which peptides comprises the amino acid sequence PHSRN (SEQ ID NO:1), wherein said sequence may contain additional amino acids added to the amino terminus, the carboxyl terminus, or both the amino and carboxyl termini.

2. The macromolecule of claim 1, wherein said dendrimer comprises polylysine.

3. A method of treating a wound on a subject comprising administering to said subject the macromolecule of claim 1.

4. A macromolecule comprising a dendrimer, said dendrimer comprising branches, said branches attached to at least one peptide consisting of an amino acid sequence PHSRN (SEQ ID NO:1).

5. The macromolecule of claim 4, wherein said at least one peptide is acetylated at the amino-terminus.

6. The macromolecule of claim 4, wherein said dendrimer comprises polylysine.

7. A method of treating a wound on a subject comprising administering to said subject the macromolecule of claim 4.


SEQUENCE LISTING (109 sequences)

SEQ ID NO: 1 - protein, 5 aa, Artificial Sequence
  Synthetic
PHSRN

SEQ ID NO: 2 - protein, 5 aa, Artificial Sequence
  Synthetic
IKVAV

SEQ ID NO: 3 - protein, 6 aa, Artificial Sequence
  Synthetic
PHSRNS

SEQ ID NO: 4 - protein, 7 aa, Artificial Sequence
  Synthetic
PHSRNSI

SEQ ID NO: 5 - protein, 8 aa, Artificial Sequence
  Synthetic
PHSRNSIT

SEQ ID NO: 6 - protein, 9 aa, Artificial Sequence
  Synthetic
PHSRNSITL

SEQ ID NO: 7 - protein, 10 aa, Artificial Sequence
  Synthetic
PHSRNSITLT

SEQ ID NO: 8 - protein, 11 aa, Artificial Sequence
  Synthetic
PHSRNSITLTN

SEQ ID NO: 9 - protein, 12 aa, Artificial Sequence
  Synthetic
PHSRNSITLTNL

SEQ ID NO: 10 - protein, 13 aa, Artificial Sequence
  Synthetic
PHSRNSITLTNLT

SEQ ID NO: 11 - protein, 14 aa, Artificial Sequence
  Synthetic
PHSRNSITLTNLTP

SEQ ID NO: 12 - protein, 15 aa, Artificial Sequence
  Synthetic
PHSRNSITLTNLTPG

SEQ ID NO: 13 - protein, 18 aa, Artificial Sequence
  Synthetic
PEHFSGRPREDRVPHSRN

SEQ ID NO: 14 - protein, 17 aa, Artificial Sequence
  Synthetic
EHFSGRPREDRVPHSRN

SEQ ID NO: 15 - protein, 16 aa, Artificial Sequence
  Synthetic
HFSGRPREDRVPHSRN

SEQ ID NO: 16 - protein, 15 aa, Artificial Sequence
  Synthetic
FSGRPREDRVPHSRN

SEQ ID NO: 17 - protein, 14 aa, Artificial Sequence
  Synthetic
SGRPREDRVPHSRN

SEQ ID NO: 18 - protein, 13 aa, Artificial Sequence
  Synthetic
GRPREDRVPHSRN

SEQ ID NO: 19 - protein, 12 aa, Artificial Sequence
  Synthetic
RPREDRVPHSRN

SEQ ID NO: 20 - protein, 11 aa, Artificial Sequence
  Synthetic
PREDRVPHSRN

SEQ ID NO: 21 - protein, 10 aa, Artificial Sequence
  Synthetic
REDRVPHSRN

SEQ ID NO: 22 - protein, 9 aa, Artificial Sequence
  Synthetic
EDRVPHSRN

SEQ ID NO: 23 - protein, 8 aa, Artificial Sequence
  Synthetic
DRVPHSRN

SEQ ID NO: 24 - protein, 7 aa, Artificial Sequence
  Synthetic
RVPHSRN

SEQ ID NO: 25 - protein, 6 aa, Artificial Sequence
  Synthetic
VPHSRN

SEQ ID NO: 26 - protein, 28 aa, Artificial Sequence
  Synthetic
PEHFSGRPREDRVPHSRNSITLTNLTPG

SEQ ID NO: 27 - protein, 5 aa, Artificial Sequence
  Synthetic
PPSRN

SEQ ID NO: 28 - protein, 5 aa, Artificial Sequence
  Synthetic
HHSRN

SEQ ID NO: 29 - protein, 5 aa, Artificial Sequence
  Synthetic
HPSRN

SEQ ID NO: 30 - protein, 5 aa, Artificial Sequence
  Synthetic
PHTRN

SEQ ID NO: 31 - protein, 5 aa, Artificial Sequence
  Synthetic
HHTRN

SEQ ID NO: 32 - protein, 5 aa, Artificial Sequence
  Synthetic
HPTRN

SEQ ID NO: 33 - protein, 5 aa, Artificial Sequence
  Synthetic
PHSNN

SEQ ID NO: 34 - protein, 5 aa, Artificial Sequence
  Synthetic
HHSNN

SEQ ID NO: 35 - protein, 5 aa, Artificial Sequence
  Synthetic
HPSNN

SEQ ID NO: 36 - protein, 5 aa, Artificial Sequence
  Synthetic
PHTNN

SEQ ID NO: 37 - protein, 5 aa, Artificial Sequence
  Synthetic
HHTNN

SEQ ID NO: 38 - protein, 5 aa, Artificial Sequence
  Synthetic
HPTNN

SEQ ID NO: 39 - protein, 5 aa, Artificial Sequence
  Synthetic
PHSKN

SEQ ID NO: 40 - protein, 5 aa, Artificial Sequence
  Synthetic
HHSKN

SEQ ID NO: 41 - protein, 5 aa, Artificial Sequence
  Synthetic
HPSKN

SEQ ID NO: 42 - protein, 5 aa, Artificial Sequence
  Synthetic
PHTKN

SEQ ID NO: 43 - protein, 5 aa, Artificial Sequence
  Synthetic
HHTKN

SEQ ID NO: 44 - protein, 5 aa, Artificial Sequence
  Synthetic
HPTKN

SEQ ID NO: 45 - protein, 5 aa, Artificial Sequence
  Synthetic
PHSRR

SEQ ID NO: 46 - protein, 5 aa, Artificial Sequence
  Synthetic
HHSRR

SEQ ID NO: 47 - protein, 5 aa, Artificial Sequence
  Synthetic
HPSRR

SEQ ID NO: 48 - protein, 5 aa, Artificial Sequence
  Synthetic
PHTRR

SEQ ID NO: 49 - protein, 5 aa, Artificial Sequence
  Synthetic
HHTRR

SEQ ID NO: 50 - protein, 5 aa, Artificial Sequence
  Synthetic
HPTRR

SEQ ID NO: 51 - protein, 5 aa, Artificial Sequence
  Synthetic
PHSNR

SEQ ID NO: 52 - protein, 5 aa, Artificial Sequence
  Synthetic
HHSNR

SEQ ID NO: 53 - protein, 5 aa, Artificial Sequence
  Synthetic
HPSNR

SEQ ID NO: 54 - protein, 5 aa, Artificial Sequence
  Synthetic
PHTNR

SEQ ID NO: 55 - protein, 5 aa, Artificial Sequence
  Synthetic
HHTNR

SEQ ID NO: 56 - protein, 5 aa, Artificial Sequence
  Synthetic
HPTNR

SEQ ID NO: 57 - protein, 5 aa, Artificial Sequence
  Synthetic
PHSKR

SEQ ID NO: 58 - protein, 5 aa, Artificial Sequence
  Synthetic
HHSKR

SEQ ID NO: 59 - protein, 5 aa, Artificial Sequence
  Synthetic
HPSKR

SEQ ID NO: 60 - protein, 5 aa, Artificial Sequence
  Synthetic
PHTKR

SEQ ID NO: 61 - protein, 5 aa, Artificial Sequence
  Synthetic
HHTKR

SEQ ID NO: 62 - protein, 5 aa, Artificial Sequence
  Synthetic
HPTKR

SEQ ID NO: 63 - protein, 5 aa, Artificial Sequence
  Synthetic
PHSRK

SEQ ID NO: 64 - protein, 5 aa, Artificial Sequence
  Synthetic
HHSRK

SEQ ID NO: 65 - protein, 5 aa, Artificial Sequence
  Synthetic
HPSRK

SEQ ID NO: 66 - protein, 5 aa, Artificial Sequence
  Synthetic
PHTRK

SEQ ID NO: 67 - protein, 5 aa, Artificial Sequence
  Synthetic
HHTRK

SEQ ID NO: 68 - protein, 5 aa, Artificial Sequence
  Synthetic
HPTRK

SEQ ID NO: 69 - protein, 5 aa, Artificial Sequence
  Synthetic
PHSNK

SEQ ID NO: 70 - protein, 5 aa, Artificial Sequence
  Synthetic
HHSNK

SEQ ID NO: 71 - protein, 5 aa, Artificial Sequence
  Synthetic
HPSNK

SEQ ID NO: 72 - protein, 5 aa, Artificial Sequence
  Synthetic
PHTNK

SEQ ID NO: 73 - protein, 5 aa, Artificial Sequence
  Synthetic
HHTNK

SEQ ID NO: 74 - protein, 5 aa, Artificial Sequence
  Synthetic
HPTNK

SEQ ID NO: 75 - protein, 5 aa, Artificial Sequence
  Synthetic
PHSKK

SEQ ID NO: 76 - protein, 5 aa, Artificial Sequence
  Synthetic
HHSKK

SEQ ID NO: 77 - protein, 5 aa, Artificial Sequence
  Synthetic
HPSKK

SEQ ID NO: 78 - protein, 5 aa, Artificial Sequence
  Synthetic
PHTKK

SEQ ID NO: 79 - protein, 5 aa, Artificial Sequence
  Synthetic
HHTKK

SEQ ID NO: 80 - protein, 5 aa, Artificial Sequence
  Synthetic
HPTKK

SEQ ID NO: 81 - protein, 3 aa, Artificial Sequence
  Synthetic
RGD

SEQ ID NO: 82 - protein, 6 aa, Artificial Sequence
  Synthetic
PHSRNC

SEQ ID NO: 83 - protein, 6 aa, Artificial Sequence
  Synthetic
GRGDSP

SEQ ID NO: 84 - protein, 6 aa, Artificial Sequence
  Synthetic
GRGESP

SEQ ID NO: 85 - protein, 5 aa, Artificial Sequence
  Synthetic
PHSCN

SEQ ID NO: 86 - protein, 5 aa, Artificial Sequence
  Synthetic
PHSCN

SEQ ID NO: 87 - protein, 5 aa, Artificial Sequence
  Synthetic
CHSRN

SEQ ID NO: 88 - protein, 5 aa, Artificial Sequence
  Synthetic
PCSRN

SEQ ID NO: 89 - protein, 5 aa, Artificial Sequence
  Synthetic
PHCRN

SEQ ID NO: 90 - protein, 5 aa, Artificial Sequence
  Synthetic
PHSRC

SEQ ID NO: 91 - protein, 5 aa, Artificial Sequence
  Synthetic
PHSXN

SEQ ID NO: 92 - protein, 5 aa, Artificial Sequence
  Synthetic
XHSXN

SEQ ID NO: 93 - protein, 5 aa, Artificial Sequence
  Synthetic
XXXXX

SEQ ID NO: 94 - protein, 5 aa, Artificial Sequence
  Synthetic
ICVAV

SEQ ID NO: 95 - protein, 4 aa, Artificial Sequence
  Synthetic
HSCK

SEQ ID NO: 96 - protein, 4 aa, Artificial Sequence
  Synthetic
HSCN

SEQ ID NO: 97 - protein, 4 aa, Artificial Sequence
  Synthetic
HSCR

SEQ ID NO: 98 - protein, 4 aa, Artificial Sequence
  Synthetic
HTCK

SEQ ID NO: 99 - protein, 4 aa, Artificial Sequence
  Synthetic
HTCN

SEQ ID NO: 100 - protein, 4 aa, Artificial Sequence
  Synthetic
HTCR

SEQ ID NO: 101 - protein, 4 aa, Artificial Sequence
  Synthetic
PSCK

SEQ ID NO: 102 - protein, 4 aa, Artificial Sequence
  Synthetic
PSCN

SEQ ID NO: 103 - protein, 4 aa, Artificial Sequence
  Synthetic
PSCR

SEQ ID NO: 104 - protein, 4 aa, Artificial Sequence
  Synthetic
PTCK

SEQ ID NO: 105 - protein, 4 aa, Artificial Sequence
  Synthetic
PTCN

SEQ ID NO: 106 - protein, 4 aa, Artificial Sequence
  Synthetic
PTCR

SEQ ID NO: 107 - protein, 5 aa, Artificial Sequence
  Synthetic
HSPNC

SEQ ID NO: 108 - protein, 8 aa, Artificial Sequence
  Synthetic
PHSCNGGK

SEQ ID NO: 109 - protein, 7 aa, Artificial Sequence
  Synthetic
PHSCNGG